(19) United States
(12) Patent Application Publication
(10) Pub. No.: US 2003/0096324 A1
(43) Pub. Date: May 22, 2003

(54) Methods for differential cell counts including related apparatus and software for performing same

(76) Inventors: Mikhail Matveev, Irvine, CA (US); Andrew Attila Pal, Rancho Santa Margarita, CA (US)

Correspondence Address: Donald Bollella, Esq., Chief Patent Counsel, BURSTEIN TECHNOLOGIES, INC., 163 Technology Drive, Irvine, CA, 92618, US

(21) Appl. No.: 10/241,512

(22) Filed: Sep. 11, 2002

Related U.S. Application Data
(60) Provisional application No. 60/322,863, filed on Sep. 12, 2001.
(60) Provisional application No. 60/353,300, filed on Jan. 31, 2002.
(60) Provisional application No. 60/353,921, filed on Jan. 31, 2002.
(60) Provisional application No. 60/355,644, filed on Feb. 5, 2002.
(60) Provisional application No. 60/355,304, filed on Feb. 8, 2002.
(60) Provisional application No. 60/358,479, filed on Feb. 19, 2002.
(60) Provisional application No. 60/363,949, filed on Mar. 12, 2002.
(60) Provisional application No. 60/404,921, filed on Aug. 21, 2002.

(51) Int. Cl. G01N 33/567; G06F 19/00; G01N 33/48; G01N 33/50

(52) U.S. Cl. 435/7.21; 702/21

(57) ABSTRACT

The present invention provides an optical method, system and software for imaging cells, in particular blood cells. In one embodiment, laboratory samples containing blood cells are deposited onto bio-discs, which are specially manufactured discs with mixing chambers that contain specific antigens to lock down various components of the blood cells. Once in the optical drive, the disc is spun and the samples and antigens are mixed with other solutions. Electromagnetic beams are then directed at the bio-disc to interact with the samples at specific capture zones and the resulting beams are collected by a detector. The information contained in the beams is then sent to a processor that produces a digital image. Various image processing methods such as binarization, background uniformization, normalization and filtering are performed to enhance cells in the investigational data for accurate counting. Other techniques are designed to correct for irregularities such as bubbles and dim cells.

### CROSS-REFERENCE TO RELATED APPLICATIONS

The present application claims the benefit of priority from U.S. Provisional Pat. App. Serial No. 60/322,863 filed Sep. 12, 2001; U.S. Provisional Pat. App. Serial No. 60/353,300 filed Jan. 31, 2002; U.S. Provisional Pat. App. Serial No. 60/353,921 also filed Jan. 31, 2002; U.S. Provisional Pat. App. Serial No. 60/355,644 filed Feb. 5, 2002; U.S. Provisional Pat. App. Serial No. 60/355,304 filed Feb. 8, 2002; U.S. Provisional Pat. App. Serial No. 60/358,479 filed Feb. 19, 2002; U.S. Provisional Pat. App. Serial No. 60/363,949 filed Mar. 12, 2002; and U.S. Provisional Pat. App. Serial No. 60/404,921 filed Aug. 21, 2002. These applications and the disclosures provided therein are hereby fully incorporated herein by reference.

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND OF THE INVENTION

1. Field of the Invention

This invention relates to signal detection apparatus, data processing methods, and related computer software and assay algorithms. The present invention is more particularly directed to imaging biological samples such as cellular samples and analyzing the collected images. More specifically, but without restriction to the particular embodiments hereinafter described in accordance with the best mode of practice, this invention relates to methods for differential cell counts including leukocytes and the use of optical bio-discs for performing such cell counts.

2. Discussion of the Related Art

A number of research and diagnostic situations require isolation and analysis of specific cells from a mixture of cells. The source of such mixtures may include blood, spinal fluid, bone marrow, tumor homogenates, lymphoid tissue, and other samples containing cellular material.

A complete blood count (CBC) is a collection of tests including hemoglobin, hematocrit, mean corpuscular hemoglobin, mean corpuscular hemoglobin concentration, mean corpuscular volume, platelet count, and white blood cell count. The most commonly used clinical test is the total CBC counts that are routinely used for assessment of health and for clinical diagnosis, treatment, and follow-up.

White blood cells (WBCS) protect the body by fighting infection and attacking foreign material. A differential white blood cell count determines the number of white blood cells and the percentage of each type of white blood cell in a person's blood. WBC or leukocyte count provides a clue to the presence of illness. These tests are included in general health examinations and help investigate a variety of illnesses, including infection, allergy, and leukemia. When extra white cells are needed, the bone marrow increases production.

There are five types of white cells, each with different functions: neutrophils, lymphocytes, monocytes, eosinophils, and basophils. The differential reveals if these cells are present in a normal distribution, or if one cell type is increased or decreased. In a normal healthy person, typically the WBC counts are 4,000 to 10,800 cells per microliter (μl). Factors such as exercise, stress, and disease can affect these values. This information helps diagnose specific types of illness. A high WBC may indicate infection, leukemia, or tissue damage. There is increased risk of infection if it falls below 1,000 cells per microliter. Conditions or medications that weaken the immune system, such as AIDS or chemotherapy, cause a decrease in white cells. Recovery from illness can be monitored by the white cell count. Counts continuing to rise or fall to abnormal levels indicate a worsening condition; counts returning to normal indicate improvement.

Leukocyte differential testing is essential to gather information beyond that obtainable from the leukocyte count itself. Leukocyte differential count is used to evaluate newly suspected infection or fever (even if the CBC is normal), suspicion of a disorder associated with abnormalities, an abnormal leukocyte count, suspected leukemia, other abnormalities such as eosinophilia, monocytosis, basophilia. Repeated testing for leukocyte or leukocyte differential may be performed in the presence of severe leukopenia (e.g., secondary to drug therapy). During treatment, for example chemotherapy or radiation therapy, blood counts are very important to determine if the treatment is depleting healthy blood cells in addition to cancerous cells.

Differential leukocyte counts are determined by computerized cell counting equipment. The machine determines the total count and the percentages of the five major white cell types. In normal individuals, there are a majority of neutrophils (50-60%), followed by lymphocytes (20-40%), then monocytes (2-9%), with a few eosinophils (1-4%) and basophils (0.5-2%).

Within the category of lymphocytes there are further sub-types of cells. For example, lymphocytes can be broadly divided into T-cells (thymus-derived lymphocytes) and B-cells (bursal-equivalent lymphocytes), which are largely responsible for cell-mediated and humoral immunity respectively. Although morphological characteristics have been used to classify groups within the leukocytes, morphology alone has proved inadequate in distinguishing the many functional capabilities of lymphocyte sub-types. To distinguish lymphocytes with various functions, techniques including analysis by rosetting, immuno-fluorescence microscopy, enzyme histochemistry, and recently, monoclonal antibodies against unique cell surface markers have been developed.

Neutrophils are important for fighting infection. When neutrophil numbers fall below 1,000 cells per microliter the condition is called neutropenia. Lymphoma treatment can cause neutropenia. Obesity and smoking increase neutrophil count. Lymphocytes are divided into B (bone marrow matured) and T (thymus matured) lymphocytes. When the lymphocyte count falls below 1,500 cells per microliter for adults or 3,000 cells per microliter in children the condition is called lymphocytopenia. Lymphomas can cause lymphocytopenia.

Platelets (thrombocytes) are cell-like particles that stop bleeding by gathering at a site where bleeding is occurring. They then activate and clump together to stop bleeding and promote clotting. Platelet counts increase during strenuous activity, if the patient has myleoproliferative disorders including infection, inflammation, malignancy, and if the spleen has been removed. An excess number of platelets is called thrombocythemia.

The number of platelets in a standard sample of blood typically is 133,000 to 333,000 platelets per microliter (μl). An excess number of platelets is called thrombocythemia. Above normal platelet counts may be due to a reactive response or bone marrow failure. Reactive responses are typically caused by bleeding, infection, neoplasia, and myeloproliferative disorders. Bone marrow failure usually involves loss of blood cells known as pancytopenia. On the other hand, decreased platelet counts are due to immune thrombocytopenia. Thrombocytopenia occurs if the platelet count fall below 30,000, which results in abnormal bleeding. Counts below 5,000 are considered life threatening.

A CBC may be done by commercially available manual or electronic instruments that measure hemoglobin level, hematocrit, total leukocyte, and erythrocyte count. Variations may include a platelet count, a leukocyte differential count, and cellular indices. The hematology analyzers are fully automated and results are accurate for cell counts, types of cells in body fluids like CSF, pleural fluid, ascetic fluid, pericardial fluid, and gastric aspiration.

As compared to prior methods and systems, we have developed a simple, miniaturized, ultra-sensitive, inexpensive system for imaging and analyzing cells and their components. This system uses optical bio-discs, related detection assemblies, as well as information and signal processing methods and software.

### SUMMARY OF THE INVENTION

The present invention is directed to methods, apparatus and software for the imaging and counting of cellular matter in laboratory samples. Embodiments of the present invention create digital images of cells in samples and perform computational analysis on the images. The present invention images cells, in particular blood cells, inclusive of the parasites and pathogens that infest the blood and other biological fluids. In other assays, the imaging is performed on beads (bead-based assays), agglutinated matter, precipitate (enzyme reaction), or other biological reporters being of a size that is detectable by the incident beam of the optical system in the present invention. This system uses optical bio-discs, related detection assemblies, as well as information and signal processing methods and software.

The present invention is also directed to bio-discs, bio-drives, and related methods. This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Serial No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 20, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed November 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Serial No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 17, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002. All of these applications are herein incorporated by reference in their entireties. They thus provide background and related disclosure as support hereof as if fully repeated herein.

One method of the present invention for performing assays is based upon the principle of optical imaging of blood cells in special channels located on the optical bio-disc. Approximately seven microliters of whole blood is injected into specially designed channels on the disc. The images are analyzed with cell recognition software that identifies these various leukocyte sub-types and generates a white cell differential count. The method is based on specific cell capture using cell specific antibodies against specific cell. In this particular case, antibodies directed against lymphocytes (CD2, CD19), monocytes (CD14), eosinophils (CD15) and so on. These leukocyte sub-type specific antibodies are assembled or attached to the solid surface within a bio-disc that includes a flow chamber.

A bio-disc drive assembly is employed to rotate the disc, read and process any encoded information stored on the disc, and analyze the cell capture zones in the flow chamber of the bio-disc. The bio-disc drive is provided with a motor for rotating the bio-disc, a controller for controlling the rate of rotation of the disc, a processor for processing return signals from the disc, and analyzer for analyzing the processed signals. The rotation rate is variable and may be closely controlled both as to speed, direction, and time of rotation. The bio-disc may also be utilized to write information to the bio-disc either before, during, or after the test material in the flow chamber and target zones is interrogated by the read beam of the drive and analyzed by the analyzer. The bio-disc may include encoded information for controlling the rotation of the disc, providing processing information specific to the type of immunotyping assay to be conducted, and for displaying any desired results on a monitor associated with the bio-drive.

Differential cell count protocol in general and in particular differential white blood cell counting protocol is developed for CD, CD-R, or DVD formats, modified versions of these formats, and alternatives thereto. The read or interrogation beam of the drive detects the various cells in the analysis sample and generates images that can be analyzed with differential cell counter software.

Microscopic methods or sophisticated cell counters are essential to perform these laborious cell-counting assays. In other assays conducted according to the invention, the cell could instead be a bead (bead-based assays), agglutinated matter, precipitate (enzyme reaction), or other biological reporters being of a size that is detectable by the incident beam of the optical system in the present invention.

The present methods use optical bio-discs and related disc assemblies. Optical images of the various leukocyte sub-types free in the analysis chamber or those captured by specific antibody methods are generated and analyzed by cell recognition software programs that identify the various cellular elements in the blood or other body fluids by their light scattering properties. The present methods do not require any processing of the sample prior to analysis like cell staining, RBC elimination and other laborious protocols. These methods include microscopic analysis or cell detection in a CD-type reader, DVD-type reader, or other optical disc reader using a top detector, bottom detector, event counter, or cell counter.

The following summary relating to cluster designation analysis, such as obtaining a CD4/CD8 ratio, represents one particular group of related assays amenable to application of the present methods, apparatus, systems, and disc assemblies.

Disc Preparation: Gold reflective discs or transmissive discs are cleaned using an air gun to remove any dust particles. The disc is rinsed twice with iso-propanol, using the spin coater. A 2% polystyrene is spin coated on the disc to give a relatively thick coating throughout.

Deposition of Chemistry: One embodiment includes a three step deposition protocol that incubates: streptavidin, 30 minute incubated; biotinylated first antibody incubated for 60 minutes; and second capture antibody incubated for 30 minutes. All the steps are preferably performed at room temperature in a humidity chamber using stringent washing and drying steps between depositions.

Briefly, 1 μl of 1 mg/ml streptavidin in phosphate buffered saline is layered over each window and incubated for 30 minutes. Excess streptavidin is rinsed off using distilled water and the disc is dried. Biotinylated IgG-dextran complex is prepared by combining equal volumes of biotinylated IgG (125 μg/ml in PBS) and aldehyde-activated dextran (200 μg/ml). Dextran-aldehyde biotinylated-lgG complex is layered over streptavidin in each capture window and incubated for 60 minutes or overnight in a refrigerator. Excess reagent is rinsed off and the disc spun-dry. Specific barcode capture patterns are created by layering capture antibodies on designated spots on the bio-disc slot. For a differential count, anti-neutrophil (CD128 or others), anti-lymphocyte (CD2, CD19, CD56, and others), anti-eosinophil (CD15), anti-monocyte (CD14), anti-basophil (CD63) and anti-platelets (CD32 and CD151) are layered in designated spot of each slot. Table 1, below, list examples of variations of capture patterns for capture layer assembly. Incubate for 30 minutes or overnight in the refrigerator. Assemble the disc using a 25 μm, 50 μm or 100 μm (50 μm channel requires twice the volume of sample as that needed for 25 μm chamber), straight, U-shaped, or other channel formats and a clear (for use with the top detector) or reflective cover disc (for use with the bottom detector).

TABLE 1
Capture Layer Assembly and Variations

| Window | 1 | 2 | 3 | 4 | 5 | 6 |
|---|---|---|---|---|---|---|
| 1^{st} Layer | Polystyrene | Polystyrene | Polystyrene | Polystyrene | Polystyrene | Polystyrene |
| (Active Layer) | | | | | | |
| 2^{nd} Layer | | Streptavidin | Streptavidin | Streptavidin | Streptavidin | Streptavidin |
| Secondary | | B-anti- | B-anti- | B-anti- | B-anti- | B-anti- |
| Antibody | | Mouse | Mouse | Mouse | Mouse | Mouse |
| | | IgG + | IgG + | IgG + | IgG + | IgG + |
| | | DCHO | DCHO | DCHO | DCHO | DCHO |
| Primary | Reference | Lymphocyte | Neutrophil | Eosinophil | Basophil | Monocyte |
| Antibody | Dot | Specific | Specific | Specific | Specific | Specific |
| | | antibody | antibody | antibody | antibody | antibody |

Leak-Checking the Disc: Since blood, a biohazardous material, is typically being analyzed, these discs are leak checked to make sure none of the chambers leak during spinning of the disc with the sample in situ. Each channel is filled with StabilGuard, a blocking agent, and blocked for at an hour. The disc is spun at 5,000 rpm for 5 minutes and inspected for leaks and disc stability. After checking for leaks, the disc is placed in a vacuum chamber for 24 hours. After vacuuming, the chambers filled with phosphate buffered saline (PBS) buffer, or alternatively left empty, are placed in a vacuum pouch and stored under refrigeration until use.

Isolation of Buffy-coat Layer from Whole Blood: Buffy coat is prepared by centrifuging venous blood with an anti-coagulant like ethylene diamine tetraacetic acid (EDTA) or acid citrate dextran (ACD) in a centrifuge tube for 15 minutes at 1,500×g. White cells form a layer at the interface of the plasma and the red blood cells called the buffy coat layer. The plasma is carefully removed using a fine pipette and then the buffy coat layer is collected. An alternate way to obtain the buffy coat from the blood without centrifugation is to allow the blood to sediment with sedimentation-enhancing agents such as fibrinogen, dextran, gum acacia, Ficoll or methylcellulose. Boyum's reagent (methylcellulose and sodium metrizoate) is particularly suitable for obtaining leukocyte preparation without any red cell contamination.

Assay on Disc—Description of Base Technology: One preferred embodiment of the differential white cell count disc test includes three individual components, (1) base disc including the chemistry, (2) channel layer, and (3) cover disc.

Buffy coat or whole blood (7 microliters in PBS) is injected into the disc chamber, the inlet and outlet ports of the chamber are sealed with closure tabs and the disc is incubated for 15 minutes at room temperature. For the first method, a given area (e.g., one millimeter square in area) on the disc is scanned using the standard 780 nm laser of the optical drive with the top or bottom detector. The cell recognition software according to the present invention is automated to give a differential count from the captured image which is equal to a millimeter square and the values obtained are extrapolated to determine counts per cubic milliliter of whole blood. And for the second barcode method, the disc is scanned using the standard 780 nm laser to image the capture zone (lymphocytes, neutrophils, basophils, eosinophils, monocytes, and platelets). The cell recognition software of this invention performs, inter alia, the following routines: (a) centrifuge the disc to spin off excess unbound cells, (b) image defined areas in each specific cell capture zones, (c) process data that includes counting the specifically captured cells in each capture zone, and (d) derive the numbers of different sub-sets of leukocytes per cubic milliliter of whole blood.

According to one aspect of the present invention, during the processing step, the recognition software reads across each capture zone and marks cells as it encounters. In other assays, the cell could instead be a bead (bead-based assays), agglutinated matter, precipitate (enzyme reaction), or other biological reporters being of a size that is detectable by the incident beam of the optical system in the present invention. Following processing data from each capture zone, the software displays the number lymphocytes, neutrophils, basophils, eosinophils, monocytes, and platelets zones per micro-liter or cubic milliliter volume of blood. The entire process takes about 10-15 minutes from inserting the disc into the optical drive to obtaining and displaying the desired counts or ratios. In another embodiment of this aspect of the present invention, the electrical response is read from the capture zone and stored on disc or in memory resulting in a data file which may be post-processed for recognition purposes as described in further detail below.

Disc Specifications: The following subsections are directed to summarizing particular embodiments of some of the optical bio-discs that may be advantageously employed in conjunction with the present invention.

(A) Tracking Design: In one preferred embodiment of the present invention, the disc is a forward Wobble Set FDL21 :13707 or FDL21 :1270 coating with 300 nm of gold. On this reflective disc, oval data windows of size 2×1 mm are etched out by Lithography. U-shaped channels are used to create chambers that are 25 to 100 micrometers in height. It takes about 7 μl of sample to fill the entire chamber including the inlet and outlet ports. A 4-window/4-channel format may be preferably used. However on the transmissive disc, no data windows are etched, and the entire disc is available for use.

(B) Adhesive and Bonding: Fraylock U-shaped adhesive DBL 201 Rev C 3M94661 or straight channels are used to create the chambers.

(C) Cover Disc: Clear disc, fully reflective with 48 sample inlets with a diameter of 0.040 inches location equidistant at radius 26 mm are used.

Data Capture and Processing: The data disc is scanned and read with the software of the present invention at speed×4 and sample rate 2.67 MHz using specific cell recognition methods.

Software: The present invention further includes processing methods and related cell recognition and imaging software. This software is directed to conducting and displaying cell counts and differential cell counts. In other assays, the cell could instead be a bead (bead-based assays), agglutinated matter, precipitate (enzyme reaction), or other biological reporters being of a size that is detectable by the incident beam of the optical system in the present invention. The present software may be stored on the optical bio-disc, in the optical disc drive reader device, or alternatively only accessible by the optical reader from a secured server. This server may be implemented in a computing network such as a Local Area Network (LAN), a Wide Area Network (WAN), or otherwise made available over the Internet under prescribed terms and conditions. Such distribution methods are disclosed in commonly assigned U.S. Provisional Application No. 60/246,824 entitled “Interactive Method and System for Analyzing Biological Samples and Processing Related Medical Information Using Specially Prepared Bio-Optical Disc, Optical Disc Drive, and Internet Connections” filed Nov. 8, 2000 and the corresponding U.S. patent application Ser. No. 09/986,078.

The materials employed to practice different preferred embodiments disclosed herein include a forward wobble gold metalized photo-resist disc, a-transmissive gold metalized disc, pipettes and tips, spin coater, centrifuge, swing-out rotor, Vacutainer™ CPT tubes with an anti-coagulant such as sodium citrate or ethylene diamine tetra acetic acid (EDTA), humidity chamber, wringer, adhesive, cover disc, clear cover disc, tape or equivalent, vacuum apparatus, yellow tips, and vacuum chamber.

In one embodiment on the present invention, laboratory samples containing blood cells are deposited on to bio-discs or in a fluidic channel formed in the disc assembly. In other assays, the cell could instead be a bead (bead-based assays), agglutinated matter, precipitate (enzyme reaction), or other biological reporters being of a size that is detectable by the incident beam of the optical system in the present invention. Bio-discs are specially manufactured CD-size discs with fluidic channels and/or mixing chambers that contain specific antigens to lock components of the blood cells in place. Because they are made of carefully layered metals and substrates, bio-discs have specific optical properties that allow electromagnetic beams to interact with the deposited test samples. Once a bio-disc is inserted into an optical drive, the drive spins the disc and in some embodiments may mix the samples along with other necessary solutions. Electromagnetic beams are directed at the bio-disc inside the drive. In one embodiment termed the reflective disc, the beams reflect off the reflective surface of the bio-disc and the detector within the optical drive collects the reflected beams. In another embodiment termed the transmissive disc, portions of the beams go through the bio-disc and are transmitted to another type of detector within the optical drive. In either case, the beams collected by the detector contain information about the laboratory samples on the bio-discs. The information is then sent to an analog to digital processor where digital data representing the electrical signal from the detector is produced. This digital data may be processed in real time, stored in memory or on disc and then processed in whole or in part as raw data, or exported into various formats including image formats. Any of these formats may be further processed by other applications or means to generate intended results. This digital data is useful for automatic, electrical, computer controlled, and/or machine counting or analysis. The digital data may also be use to produce viewable images suitable for expert hand counts, recognition, or other manual analysis. In another embodiment of the invention, the raw data, digital data, exported data that may include images are stored in an archive. Thus the methods of the present invention may generally apply to “investigational data” which as used herein includes, but is not limited to, raw detector output data, raw signal data, digital data, exported data, or exported data including images or image data. The archive provides a place where investigational data can be cataloged and, if desired, associated with other identifying information such as, for example, demographic, geographic, medical, historic, or personal data. Subsequently, groups of investigational data can be analyzed to conduct health trend studies of, for example, different population groups.

One embodiment of the invention addresses the need to count blood cells. The present invention includes processing methods and related cell recognition and imaging software. This software is directed to conducting cell counts and displaying the corresponding results. In one embodiment of the invention, various image processing methods such as binarization, background uniformization, normalization and filtering are performed to enhance the appearance of the cells in the investigational data to aid the process of cell counting. Other techniques are performed to correct the cell counts for irregularities such as trapped bubbles, cracks, and dim cells in the investigational data.

Embodiments of the present invention store the software on the optical bio-disc, in the optical disc drive reader device, or alternatively only accessible by the optical reader from a secured server. This server may be implemented in a computer networks such as a Local Area Network (LAN), a Wide Area Network (WAN), or otherwise made available over the Internet under prescribed terms and conditions. Such distribution methods are disclosed in commonly assigned U.S. application No. 09/986,078 entitled “Interactive System for Analyzing Biological Samples and Processing Related Information and the Use Thereof” filed Nov. 7, 2001 which is herein incorporated by reference.

More specifically, the present invention is directed to a method of counting cells or other investigational features. This method includes the steps of obtaining investigational data of a sample with cells, selecting an evaluation rectangle in the investigational data, enhancing the investigational data inside the evaluation rectangle, and counting cells shown in the evaluation rectangle. In one specific embodiment of this method, cell counting is performed by recognizing bright centers or alternatively dark rims.

Another aspect of the present invention is directed to a method of selecting a custom size for the evaluation rectangle.

Yet another aspect of the present invention is directed to a method of selecting a plurality of evaluation rectangles.

Still a further aspect of the present invention is directed to enhancing investigational data inside an evaluation rectangle through the steps of performing background illumination uniformization on the investigational data, performing normalization on the investigational data, and filtering the investigational data.

An additional aspect of the present invention is directed to performing background illumination uniformization on the investigational data through the steps of choosing a size for a neighborhood rectangle, picking a point in the investigational data, performing horizontal scanning to calculate a first sliding average for all neighbor points located within the neighborhood rectangle centered at the point, performing vertical scanning to calculate a second sliding average for all neighbor points located within the neighborhood rectangle centered at the point, combining the first sliding average and second sliding average to create an overall average, reassigning the original value of the point to a resultant value calculated by obtaining the difference between the overall average and the original value and adding the difference to a background value, and repeating the steps of performing horizontal scanning, performing vertical scanning, combining the two averages and reassigning the original value for all points in the investigational data.

In another aspect of the present invention, the step of performing normalization on investigational data further includes the steps of calculating an average and a standard deviation of the value all points in the investigational data, normalizing the value of all points in the investigational data using the average and standard deviation and truncating the value of some points if necessary.

According to yet another aspect of the present invention there is provided a method of filtering investigational data that includes the steps of choosing a size for a neighborhood rectangle, picking a point in the investigational data, finding all sufficiently distinct points located in the neighborhood rectangle centered at the point, reassigning the value of the point if the number of the sufficiently distinct points is greater than a pre-determined filtering criteria, and repeating the steps of finding all sufficiently distinct points and reassigning the value for all points in the investigational data.

In accordance with still a further aspect of the present invention, there is provided a processing method including the steps of removing undesirable components from the investigational data comprising the steps of selecting a threshold value, performing binarization on the investigational data using the threshold value, performing regularization on the investigational data, extracting connected components, selecting a size threshold, and removing components that fail to meet the size threshold.

Another additional aspect of the present invention is directed to a method of counting cells in investigational data by bright centers. This method includes the steps of performing convolution on the investigational data, searching for a plurality of local maxima, removing redundant local maxima from the plurality of local maxima, declaring remaining maxima to be centers of cells; and counting the centers of cells.

According to another aspect of this invention, there is provided another method of counting cells in investigational data by bright centers. This alternative method includes the steps of performing inversion on said investigational data, performing a plurality of convolutions with shifted rings, summing results from said plurality of convolutions, finding local maxima, declaring maxima to be centers of cells, and counting said centers of cells.

Furthermore, another aspect of the present invention is directed to a method including the steps of removing cells counted from investigational data that have been counted by the bright centers, counting cells by their dark dims, and adding total from the step of counting cells by recognizing bright centers to the total from the step of counting by recognizing dark rims.

Yet another aspect of the present invention includes a method of counting cells by dark rims in investigational data. This method includes the steps of performing inversion on the investigational data, performing a plurality of convolutions with shifted rings, summing results from the plurality of convolutions, declaring maxima to be centers of cells, and counting the cells.

In another aspect of the invention, the method of enhancing investigational data for the purpose of cell counting further includes the steps of performing normalizing on the investigational data, performing filtering on the investigational data, selecting a threshold number, performing binarization on the investigational data by determining if the investigational data differs from a set background value by said threshold number, performing regularization on the investigational data, extracting one-pixel wide boundaries in the investigational data, filling in areas defined by the one-pixel boundaries, and applying convolution in the filled in areas.

Another aspect of the present invention is directed to a method of obtaining a digital data of a sample with cells. This method includes the steps of (1) providing a blood sample on an optical disc surface, (2) loading the optical disc into an optical reader, (3) rotating the optical disc, and (4) directing an incident beam of electromagnetic radiation to one of the capture zones on the optical disc. The surface is provided with one or more capture zones having one or more capture agents. This method continues with the steps of (5) detecting with a detector a beam of electromagnetic radiation formed after interacting with the disc at the capture zone, (6) converting the detected beam into an analog output signal, and (7) converting the analog output signals into digital data containing cells captured at the capture zone.

According to another aspect of the present invention there is provided a method of converting an analog output to digital data. This conversion method includes the steps of sampling amplitudes of the analog signals at fixed intervals, recording the sampling amplitudes in an one-dimensional array, creating a plurality of one-dimensional arrays using the steps of sampling and recording, and combining the plurality of one-dimensional arrays to create a two-dimensional array containing digital data of the sample.

Still another aspect of the present invention is directed to a method of obtaining digital data of a sample with cells. This method includes the steps of providing a blood sample on an optical disc surface (with the surface including one or more capture zones with one or more capture agents), loading the optical disc into an optical reader, rotating the optical disc, directing an incident beam of electromagnetic radiation to one of the capture zones on the optical disc, detecting with a detector a beam of electromagnetic radiation formed after interacting with the disc at the capture zone, and converting the detected beam into an analog output signal. This particular embodiment of the present method concludes with the step of converting the analog output signals into digital data containing cells captured at the capture zone. The optical disc is constructed with a reflective layer such that light directed to the capture zone is reflected to the detector and detector is a bottom detector. In another aspect of the present invention, a top detector or a split detector is used.

Another aspect of the present invention is directed to a method of selecting evaluation rectangles. This method includes the steps of (1) finding one of a plurality of windows in the investigational data, and (2) cropping an evaluation rectangle of standard size inside the window. In one particular embodiment of this method, the step of finding one of the plurality of windows includes the steps of (a) performing compression on the investigational data, (b) performing threshold evaluation on the image, (c) performing binarization on the investigational data, (d) performing regularization on the investigational data, (e) extracting connected components from the investigational data, and (f) finding a component from the connect components that corresponds to a window.

Still yet a further aspect of the present invention is directed to a method of extracting connected components from the investigational data. This method includes the steps of assigning initial component numbers to components to all black points on the investigational data, setting an initial scanning direction, and scanning the investigational data to reassign the component numbers so that the component numbers of connected black points become the same.

And still an additional aspect of the present invention is directed to a method of selecting evaluation rectangles in investigational data from optical disc embodiments with dark spots. This method includes the steps of finding at least one of the dark spots in the investigational data, and creating an evaluation rectangle of standard size with a center located at a point found by shifting a pre-determined distance from the dark spot.

According to the display aspects of the present invention, there is provided a method of enhancing the display of an image of investigational data. This method includes the steps of performing Fast Fourier Transform on the investigational data, removing some part of the spectrum of the data in the frequency domain, and performing an inverse transform to recover a modified version of the investigational data.

Also according to the display aspects of the present invention, there is provided another method of enhancing the display of an image of investigational data. This method includes the steps of determining if the image is skewed, finding the direction of the skew, and correcting the skew of the image.

Another aspect of the present invention is directed to a method of retrieving previously stored investigational data from an archive and performing analysis on the investigational data. Such archive can catalog stored investigational data according to characteristics of patients who donated the test samples. In one aspect of the present invention, the samples matching a plurality of criteria chosen from the characteristics of patients are selected to conduct a population health trends study.

Another aspect of the present invention is directed to counting different components in white blood cell counts and displaying the counts of CD4⁺ cells and CD8⁺ cells, and a ratio of CD4 to CD8 cells.

### BRIEF DESCRIPTION OF THE DRAWING

Further objects, aspects, and methods of the present invention together with additional features contributing thereto and advantages accruing therefrom will be apparent from the following description of the preferred embodiments of the invention which are shown in the accompanying drawing, wherein:

FIG. 1 is a pictorial representation of a bio-disc system according to the present invention;

FIG. 2 is an exploded perspective view of a reflective bio-disc as utilized in conjunction with the present invention;

FIG. 3 is a top plan view of the disc shown in FIG. 2;

FIG. 4 is a perspective view of the disc illustrated in FIGS. 2 and 3 with cut-away sections showing the different layers of the disc;

FIG. 5 is an exploded perspective view of a transmissive bio-disc as employed in conjunction with the present invention;

FIG. 6 is a top plan view of the disc shown in FIG. 5;

FIG. 7 is a perspective view of the transmissive disc illustrated in FIGS. 5 and 6 with cut-way sections showing the different layers of the disc including the type of semi-reflective layer shown in FIG. 8;

FIG. 8 is a perspective view representing the disc shown in FIG. 7 with a cut-away section illustrating the functional aspects of a semi-reflective layer of the disc;

FIG. 9 is a graphical representation showing the relationship between thickness and transmission of a thin gold film;

FIG. 10A is a perspective and block diagram representation illustrating the operation of a system according to one embodiment of the present invention;

FIG. 10B shows a split detector and the cross section of a transmissive bio-disc according to an embodiment of the invention;

FIG. 11 is a partial cross sectional view taken perpendicular to a radius of the reflective optical bio-disc illustrated in FIGS. 2, 3 and 4 showing a flow channel formed therein;

FIG. 12 is a partial cross sectional view taken perpendicular to a radius of the transmissive optical bio-disc depicted in FIGS. 5, 6 and 7 showing a flow channel formed therein and a single top detector;

FIG. 13 is a partial longitudinal cross sectional view of the reflective optical bio-disc shown in FIGS. 2, 3 and 4 illustrating a wobble groove formed therein;

FIG. 14 is a partial longitudinal cross sectional view of the transmissive optical bio-disc shown in FIGS. 5, 6 and 7 illustrating a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17 is a flow chart showing the process of data collection from a bio-disc using methods of the present invention;

FIG. 18 is a pictorial graphical representation of the transformation of a sampled analog signal to a corresponding digital signal that is stored as a one-dimensional array;

FIG. 19 is a perspective view of an optical disc with an enlarged detailed view of the section indicated showing a captured white blood cell positioned relative to the tracks of an optical bio-disc yielding a signal-containing beam after interacting with an incident beam;

FIG. 20A is a graphical representation of a white blood cell positioned relative to the tracks of an optical bio-disc according to the present invention;

FIG. 20B is a series of signature traces derived from the white blood cell of FIG. 20A according to the present invention;

FIG. 21 is a graphical representation illustrating the relationship among FIGS. 21A, 21B, 21C, and 25D;

FIGS. 21A, 21B, 21C, and 21D, when taken together, are pictorial graphical representations of transformation of the signature traces from FIG. 20B into digital signals that are stored as one-dimensional arrays and combined into a two-dimensional array for data input;

FIG. 22 is a flow chart depicting the steps for data evaluation according to the processing methods and computational algorithms of the present invention;

FIG. 23 is a flow chart showing the steps involved in selecting evaluation rectangles according to an embodiment of the invention;

FIG. 24 is a graphical representation of a bio-disc with windows as displayed by the software in accordance with a particular embodiment of the invention;

FIG. 25 is a flow chart illustrating the steps involved in finding windows on investigational data collected from bio-discs with windows according to an embodiment of the invention;

FIG. 26 shows an example row from an investigational data array undergoing a process of scanning for the purpose of finding a threshold value according to an aspect of the present invention;

FIG. 27 is a flow chart showing the sub-steps involved in extracting connected components from investigational data according to another aspect of the invention;

FIG. 28 depicts the results of cropping an evaluation rectangle after finding the windows on the software display according to an embodiment of the present invention;

FIG. 29 shows an example dark spot on a disc without windows and target zones with captured cells;

FIG. 30 is a view similar to FIG. 29 showing how an example dark spot is utilized in a disc without windows to find the cells according to an embodiment of the present invention;

FIG. 31 is a flow chart showing the steps involved in performing background illumination uniformization on investigational data according to certain aspects of the invention;

FIG. 32 presents an example of investigational data before background illumination uniformization as displayed by the software of the present invention;

FIG. 33 shows an example of investigational data after background illumination uniformization as displayed by the software of the present invention;

FIG. 34 is a flow chart illustrating the steps involved in performing normalization on investigational data according to a particular implementation of the present invention;

FIG. 35 shows a graphical representation of example investigational data as displayed by the software during the step normalization;

FIG. 36 shows a graphical representation of example investigational data after normalization as displayed by the software of the present invention;

FIG. 37 is a flow chart showing the steps involved in performing filtering on example investigational data according to a preferred embodiment of the invention;

FIG. 38 presents a graphical representation of example investigational data after the filtering step as displayed by the software of the present invention;

FIG. 39 is a close-up view of the graphical representation shown in FIG. 38 with an accompanying point value graph trace;

FIG. 40 is a flow chart showing the steps involved in removing undesirable components from investigational data according to a specific embodiment of certain aspects of the present invention;

FIG. 41 shows a graphical representation of example investigational data before the removal of cracks as displayed by the software of the present invention;

FIG. 42 is a graphical representation of the example investigational data of FIG. 41 after the removal of cracks as displayed by the software of the present invention;

FIG. 43 is a flow chart showing the steps involved in marking and counting cells according to the bright center method of the invention;

FIG. 44 shows a graphical representation of example investigational data filled with cells counted by the bright center method;

FIG. 45 presents a up-close view and a value trace graph of a portion of the graphical representation shown in FIG. 44;

FIG. 46A is a flow chart showing the steps involved in marking and counting cells according to the dark rims method of the present invention;

FIG. 46B is a graphical representation of convolution with shifted rings;

FIG. 47 is a graphical representation of example investigational data in which counted cells are marked by crosses as displayed by the software of the present invention;

FIG. 48 presents an example flow chart showing the steps involved in extracting red blood cells using an algorithm utilized in different methods of the present invention;

FIG. 49 shows a graphical representation of investigational data containing red blood cells before the algorithm outlined in FIG. 48 is performed;

FIG. 50 illustrates a graphical representation of the example investigational data of FIG. 49 after the first step of the algorithm outlined in FIG. 48 is performed;

FIG. 51 depicts a graphical representation of the example investigational data of FIG. 49 after applying the second step of algorithm outlined in FIG. 48;

FIG. 52 represents visually the example investigational data of FIG. 49 after the third step of the algorithm outlined in FIG. 48 is performed;

FIG. 53 shows a graphical representation of the example investigational data of FIG. 49 after performing the fourth step of algorithm outlined in FIG. 48;

FIG. 54 illustrates a graphical representation of the example investigational data of FIG. 49 after applying the fifth step of the algorithm outlined in FIG. 48 is performed;

FIG. 55 is a close-up view showing red blood cells that are counted by the algorithm outlined in FIG. 48;

FIG. 56A is a pictorial screen shot of discrete cells before they are counted by the absolute value counting method of the present invention;

FIG. 56B is a flow chart showing the steps involved in counting cells by one embodiment of the absolute value counting method of the invention;

FIG. 57 is a pictorial screen shot of the discrete cells originally shown in FIG. 56A after performing the step of normalization and filtering according to this embodiment of the absolute value counting method of the present invention;

FIG. 58 is a pictorial screen shot of the discrete cells originally shown in FIG. 56A after applying the step of background removal and binarization in accordance with the absolute value counting method of the invention;

FIG. 59 is a pictorial screen shot of the discrete cells originally shown in FIG. 56A after the step of regularization is performed according to the absolute value counting method of the invention;

FIG. 60 is a pictorial screen shot of the discrete cells originally shown in FIG. 56A after applying the step of one-pixel wide boundary extraction in accordance with the illustrated embodiment of the absolute value counting method of the invention;

FIG. 61 is a pictorial screen shot of the discrete cells originally shown in FIG. 56A after performing the step of filling in components according to the absolute value counting method of the present invention;

FIG. 62 is a pictorial screen shot of the discrete cells originally shown in FIG. 56A after applying the step of filling in investigational data according to this particular embodiment of the absolute value counting method of the invention;

FIG. 63 is a pictorial screen shot of the discrete cells originally shown in FIG. 56A after they are counted and marked by crosses in accordance with the methods of the present invention;

FIG. 64 shows the results of the absolute value counting method applied to counting clumped and discrete red blood cells;

FIG. 65 is a flow chart showing the steps involved in performing Fast Fourier Transform on an image according to an alternative embodiment of the present invention;

FIG. 66 is a graphical representation of example investigational data before a Fast Fourier Transform is performed according to the present invention;

FIG. 67 shows a graphical representation of the example investigational data of FIG. 66 after the Fast Fourier Transform is performed;

FIG. 68 illustrates an example of a skewed graphical representation of investigational data before realignment;

FIG. 69 depicts the skew direction of the graphical representation shown in FIG. 68;

FIG. 70 shows the graphical representation of FIG. 68 after realignment;

FIG. 71A is a flow chart depicting the steps involved in correcting cell counts for bubble track situations;

FIG. 71B is a pictorial representation of correcting cell counts for bubble track situations according to another aspect of the present invention;

FIG. 71C is an example of bubble tracks through a target zone of captured red blood cells as would be seen under microscope power 5×;

FIG. 71D is an enlarged view of one of the bubble tracks and surrounding captured red blood cells of FIG. 71C as would be seen under microscope power 40×; and

FIG. 72 is a pictorial flow chart showing the analysis of a blood sample using the methods of the present invention.

### DETAILED DESCRIPTION OF THE INVENTION

The present invention is directed to methods and apparatus for the imaging and counting of cellular matters in laboratory samples. These methods and methods may be applied to imaging and counting any type of investigational features of interest on or in an optical disc. Embodiments of the present invention create investigational data of investigational features or cells in samples and perform computational analysis on the investigational data. In other assays, the cell could instead be a bead (bead-based assays), agglutinated matter, precipitate (enzyme reaction), or other biological reporters being of a size that is detectable by the incident beam of the optical system in the present invention.

In the following description, numerous specific details are set forth to provide a more thorough description of embodiments of the invention. It should be apparent, however, to one skilled in the art that the invention may be practiced without these specific details. In other instances, well known features have not been described in detail so as not to obscure the invention.

A number of embodiments for white blood cell counting using optical disc data are herein discussed in further detail. These embodiments are not limited to the imaging and counting white blood cells only, but may be readily applied to conducting counts of any type of cellular matter. This can include, but is not limited to, red blood cells, white blood cells, beads, and any other objects, both biological and non-biological, that produce similar optical signatures that can be detected by an optical reader. In other assays, the investigational features of interest, rather than being a cells, could instead be beads (bead-based assays), agglutinated matter, precipitate (enzyme reaction), or other biological reporters having a size that is detectable by the incident beam of the optical system in the present invention. Some of the modifications needed to use the present invention on matter other than white blood cells are described in further detail below in the discussion of cell counting.

In the following discussion, two main sections are presented to illustrate the data collection and data analysis aspects on the present invention. The first section presents a detailed description of the apparatus, methods, and algorithms used to collect investigational data from the laboratory samples and transform such investigational data into an array-based storage. The second section presents a detailed description of the methods and algorithms directed to analysis of the investigational data. Following the two sections, a section on a method of conducting a white blood cell count assay is given.

I. Data Collection

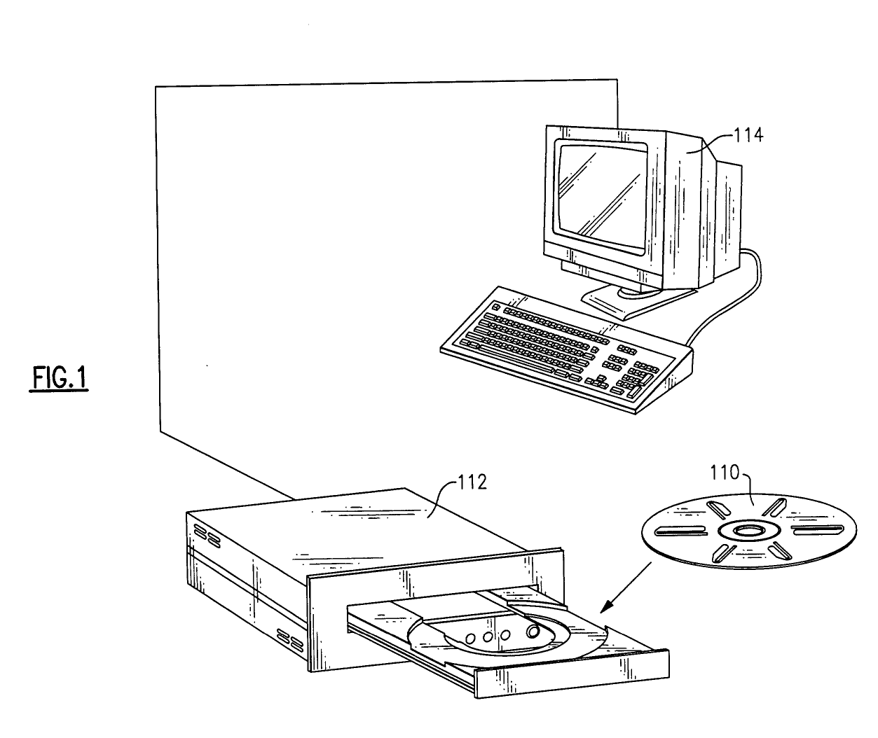

Embodiments of the present invention involve the retrieval of investigational data from cellular matter in laboratory samples. FIG. 1 is a perspective view of an optical bio-disc 110 according to the present invention. The present optical bio-disc 110 is shown in conjunction with an optical disc drive 112 and a display monitor 114. Test samples are deposited onto designated areas on bio-disc 110. Once the bio-disc is inserted into optical disc drive 112, the disc drive is responsible for collecting information from the sample through the use of electromagnetic radiation beams that have been modified or modulated by interaction with the test samples. After the information is analyzed and processed, computer monitor 114 displays the results.

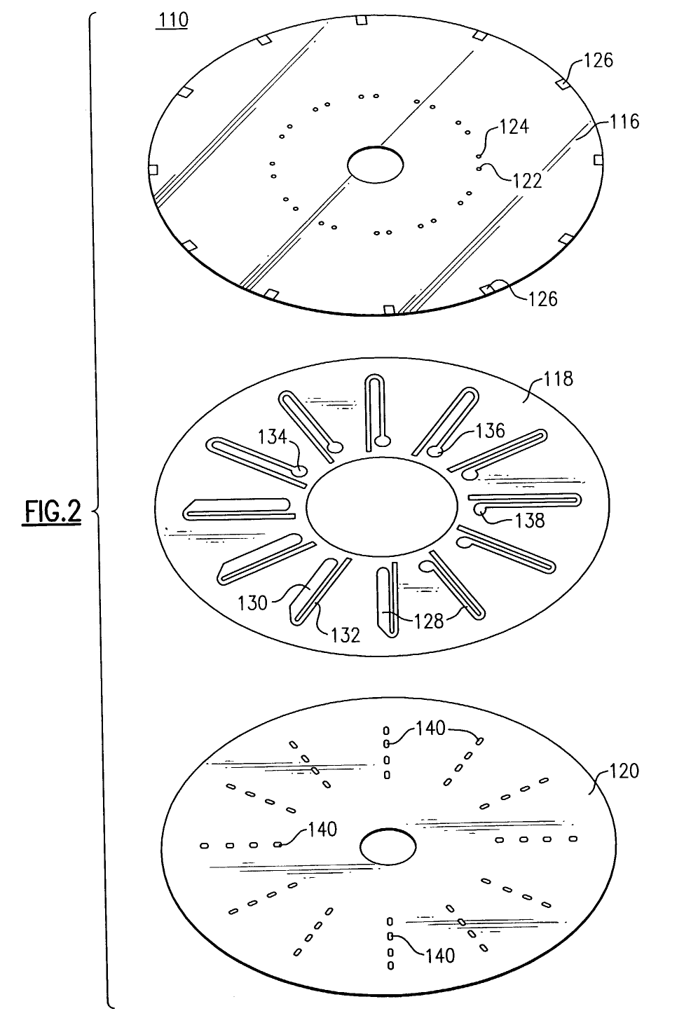

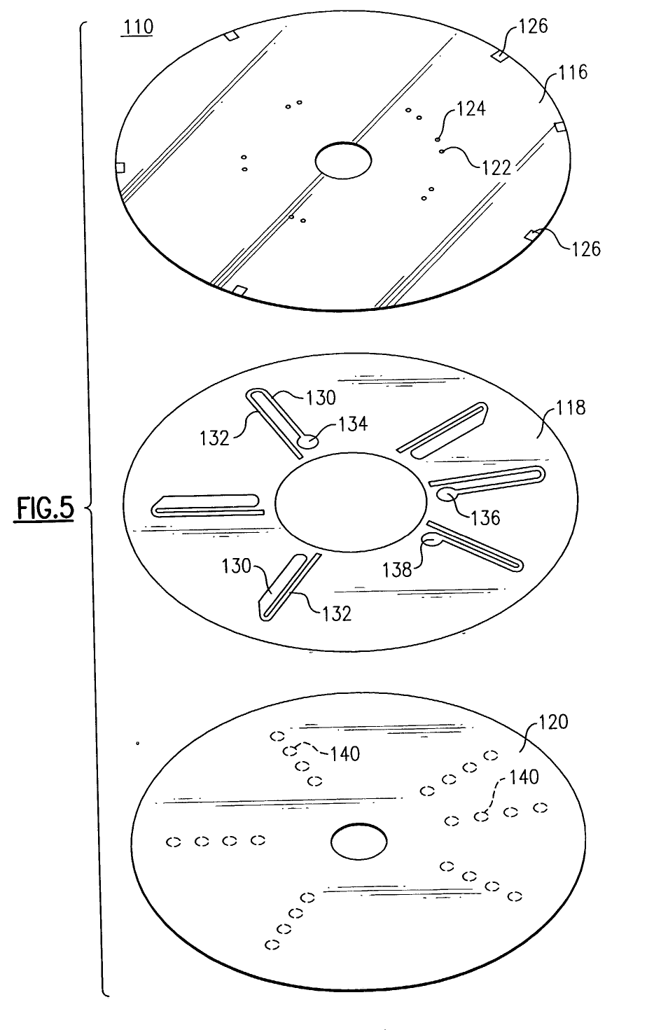

There are two main embodiments of optical bio-disc 110 that can be used in the present invention. FIGS. 2, 3 and 4 illustrate the reflective embodiment of optical bio-disc 110 while FIGS. 5, 6 and 7 illustrate the transmissive embodiment of optical bio-disc 110.

A. Reflective Embodiment

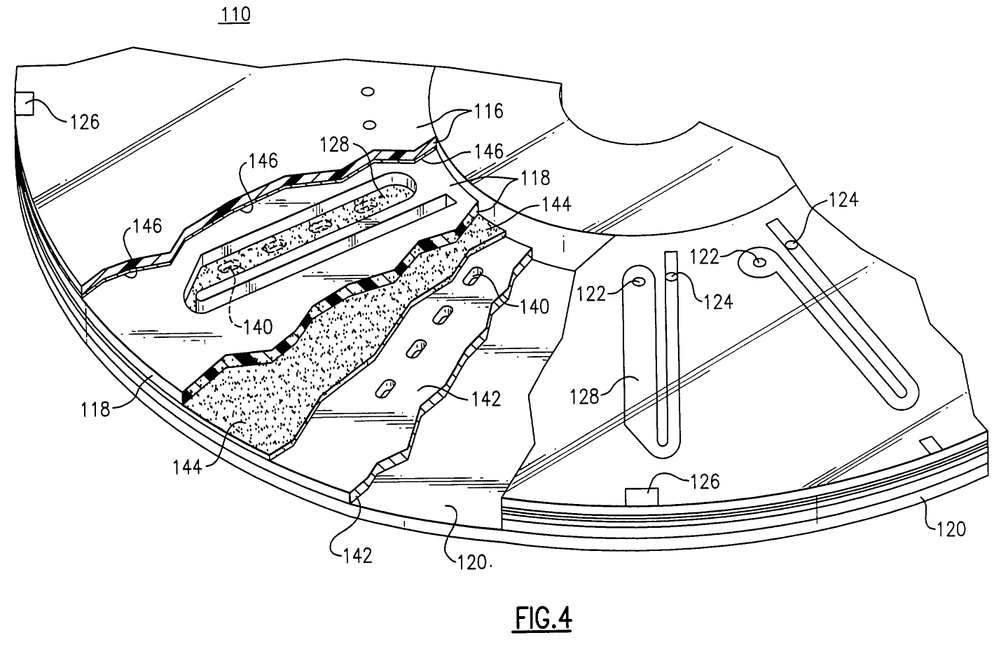

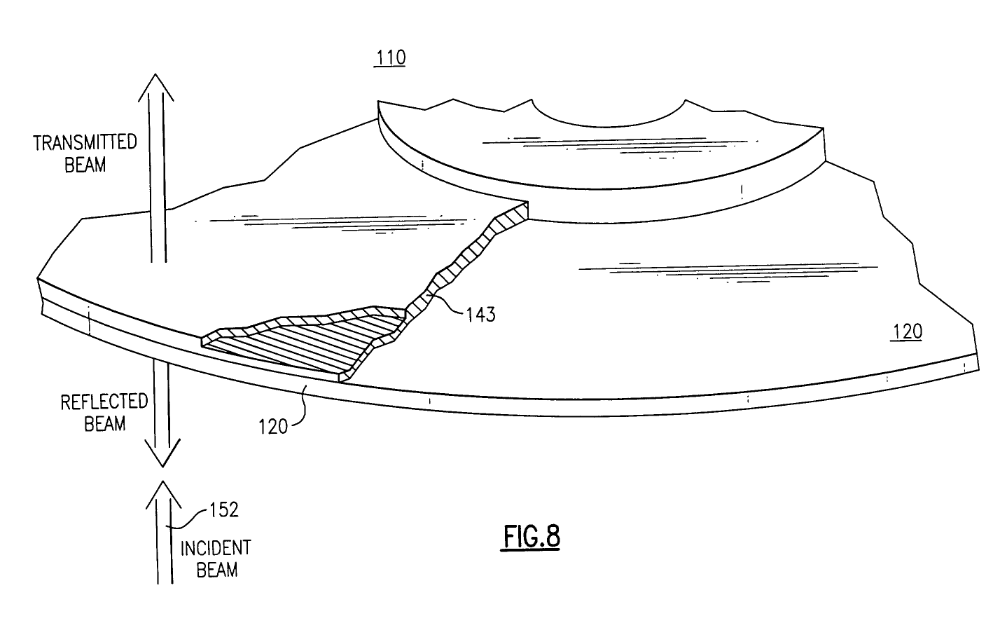

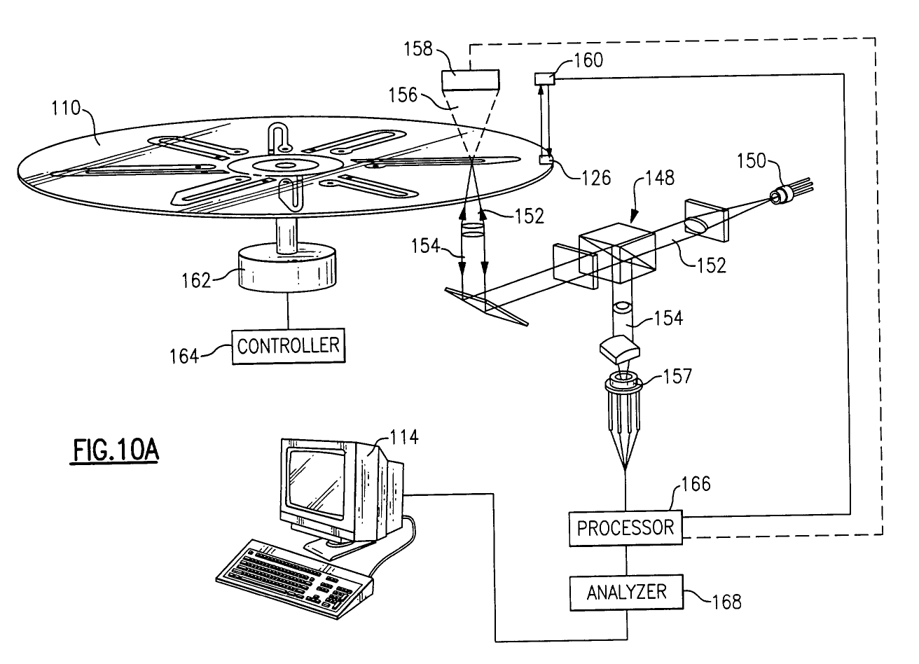

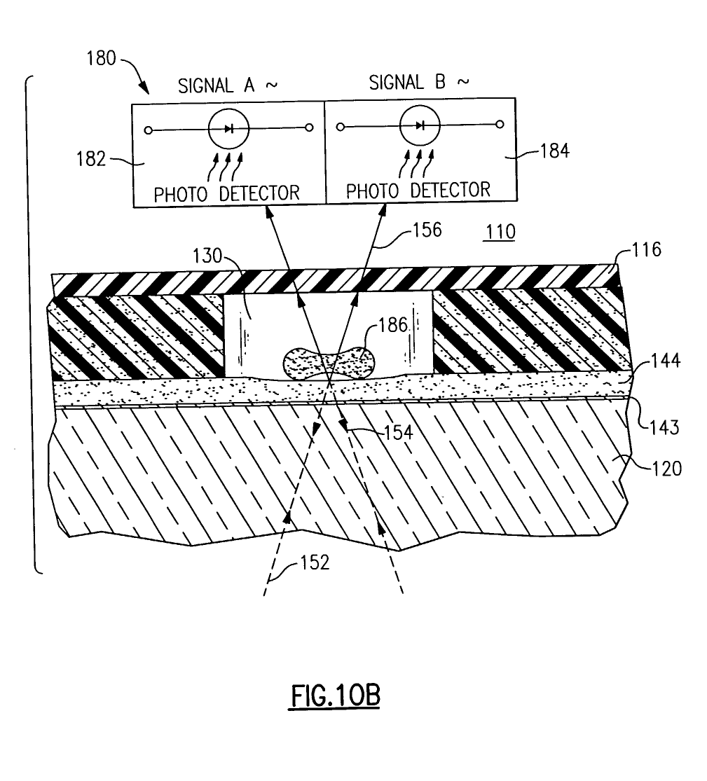

FIG. 2 is an exploded perspective view of the structural elements of one embodiment of the optical bio-disc 110. FIG. 2 is an example of a reflective zone optical bio-disc 110 (hereinafter “reflective disc”) that may be used in the present invention. The structural elements include a cap portion 116, an adhesive or channel member 118, and a substrate 120. The cap portion 116 includes one or more inlet ports 122 and one or more vent ports 124. The cap portion 116 may be formed from polycarbonate and is preferably coated with a reflective surface 146 (as better illustrated in FIG. 4) on the bottom thereof as viewed from the perspective of FIG. 2. In the preferred embodiment, trigger markings 126 are included on the surface of the reflective layer 142, FIG. 4. Trigger markings 126 may include a clear window in all three layers of the bio-disc, an opaque area, or a reflective or semi-reflective area encoded with information. The encoded information is used to send data to a processor 166 (shown in FIG. 10A) that in turn interacts with the operative functions of the interrogation or incident beam 152 shown in FIGS. 8 and 10A. The second element shown in FIG. 2 is an adhesive or channel member 118 having fluidic circuits 128 or U-channels formed therein. The fluidic circuits 128 are preferably formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated. The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

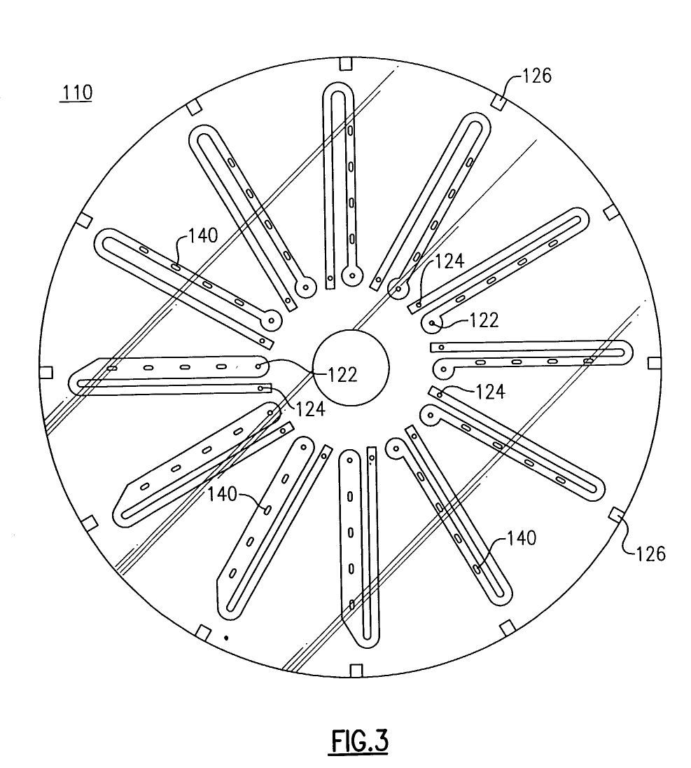

FIG. 3 is a top plan view of the optical bio-disc 110 illustrated in FIG. 2 with the reflective layer 142 on the cap portion 116 shown as transparent to reveal the fluidic circuits 128, the target or capture zones 140 and trigger markings 126 situated within the disc. Since each capture zone has one or more specific antigens to lock down different components (or different cells) in the samples, after assay processing, each capture zone inside the chamber contains a type of cells or cell components. The locking or capturing is accomplished by having one or more antigens with chemical structure that can “lock” onto a specific component of blood cells and thereby capture that specific cell. The separation of cell components is critical for performing a differential count in blood cells, for example, white blood cells. In other assays, rather than cells, the investigational features could instead be beads (bead-based assays), agglutinated matter, precipitate (enzyme reaction), or other biological reporters of a size that is detectable by the incident beam of the optical system in the present invention. The target or capture zones 140 are define the location where the electromagnetic interrogation beam will interact with the test samples.

FIG. 4 is an enlarged perspective view of the reflective zone type optical bio-disc 110 according to one embodiment of the present invention. This view includes a portion of the various layers thereof, cut away to illustrate a partial sectional view of each layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition hydrogels can be used. As illustrated in this specific embodiment, the plastic adhesive member 118 is applied over the active layer 144. The exposed section of the plastic adhesive member 118 illustrates the cut out or stamped U-shaped form that creates the fluidic circuits 128. The final structural layer in this reflective zone embodiment of the present bio-disc is the cap portion 116. The cap portion 116 includes the reflective surface 146 on the bottom thereof. The reflective surface 146 may be made from a metal such as aluminum or gold.

B. Transmissive Embodiment

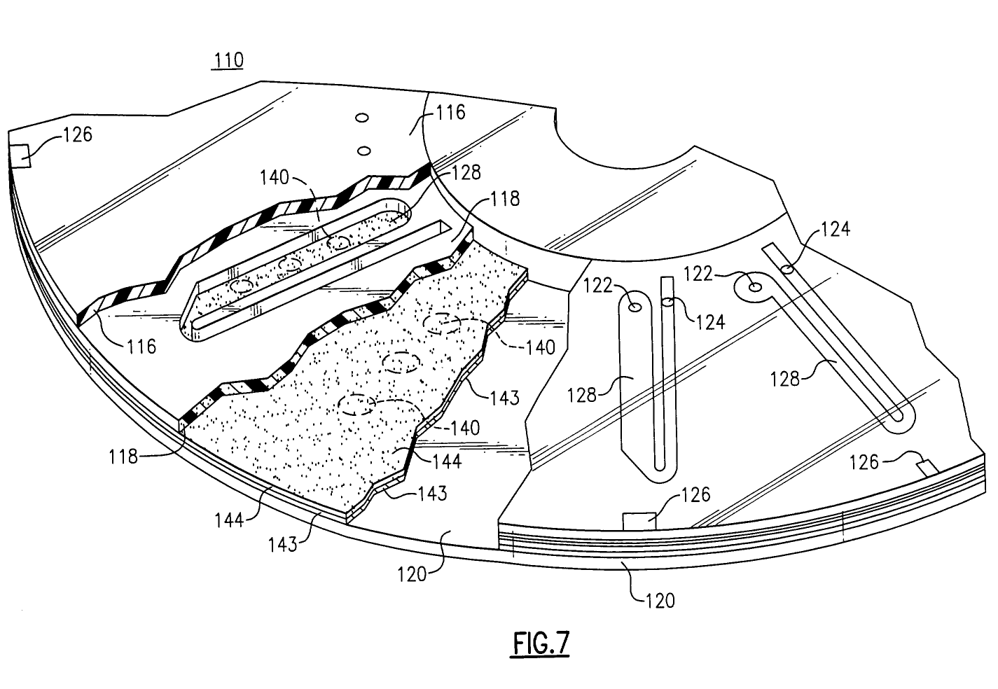

FIG. 5 is an exploded perspective view of the structural elements of a transmissive type of optical bio-disc 110 according to the present invention. The structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as best illustrated in FIGS. 7 and 8. Trigger markings 126 may include a clear window in all three layers of the bio-disc, an opaque area, or a reflective or semi-reflective area encoded with information. The encoded information is used to send data to a processor 166 (shown in FIG. 10A) that in turn interacts with the operative functions of the interrogation or incident beam 152 shown in FIGS. 8 and 10A.

The second element shown in FIG. 5 is the adhesive or channel member 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

The third element illustrated in FIG. 5 is the substrate 120, which may include the target or capture zones 140. The substrate 120 is preferably made of polycarbonate and has the thin semi-reflective layer 143 deposited on the top thereof, FIG. 8. The semi-reflective layer 143 associated with the substrate 120 of the disc 110 illustrated in FIGS. 5 and 8 is significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIG. 11. The thin semi-reflective layer 143 may be formed from a metal such as aluminum or gold.

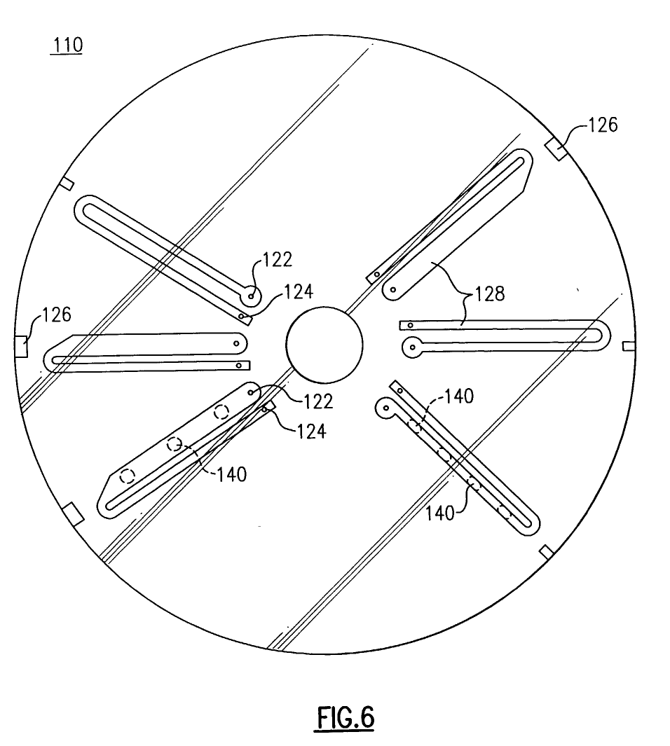

FIG. 6 is a top plan view of the transmissive type optical bio-disc 110 illustrated in FIG. 4 with the transparent cap portion 116 revealing the fluidic channels, the trigger markings 126 and the target or capture zones 140 as situated within the disc. The target or capture zones 140 are where the electromagnetic beam will interact with the test samples. After the spinning of the disc, specific components of cells in the samples are captured in different capture zones by various capture agent or antigens pre-loaded inside the chamber.

FIG. 7 is an enlarged perspective view of the optical bio-disc 110 according to the transmissive disc embodiment of the present invention. The disc 110 is illustrated with a portion of the various layers thereof cut away to illustrate a partial sectional view of each layer, substrate, coating, or membrane. FIG. 7 illustrates a transmissive disc format with the clear cap portion 116, the thin semi-reflective layer 143 on the substrate 120, and trigger markings 126. Trigger markings 126 include opaque material placed on the top portion of the cap. Alternatively the trigger markings 126 may be formed by clear, non-reflective windows etched on the thin reflective layer 143 of the disc, or any mark that absorbs or does not reflect the signal coming from the trigger detector 160, FIG. 10A. FIG. 7 also shows, the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking the entire thin semi-reflective layer 143 except the target zones 140. In this embodiment, target zones 140 may be created by silk screening ink onto the thin semi-reflective layer 143. An active layer 144 is applied over the thin semi-reflective layer 143. In the preferred embodiment, the active layer 144 is a 40 to 200 μm thick layer of 2% polystyrene. Alternatively, polycarbonate, gold, activated glass, modified glass, or modified polystyrene, for example, polystyrene-co-maleic anhydride, may be used. In addition hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128. The final structural layer in this transmissive embodiment of the present bio-disc 110 is the clear, non-reflective cap portion 116 that includes inlet ports 122 and vent ports 124.

C. Optical Properties of the Disc Embodiments

One of the main differences between the two disc embodiments is the thickness of the coating of the top layer on the optical disc. In the case of the transmissive disc, a thin semi-reflective layer 143 is deposited on the top of the substrate layer 120. In the case of the reflective disc, a substantially thicker reflective layer is deposited on top of its substrate layer 120. In the preferred embodiment illustrated by FIG. 8, the thin semi-reflective layer 143 of the transmissive disc is approximately 100 to 300 Å thick and does not exceed 400 Å. This is because the gold film layer is fully reflective at a thickness greater than 800 Å and allows for the light to transmit through the gold film at a thickness of approximately below 400 Å. As indicated below, Table 2 presents the reflective and transmissive characteristics of a gold film relative to the thickness of the film.

TABLE 2
Au film Reflection and Transmission (Absolute Values)

| Thickness | Thickness | | |
|---|---|---|---|
| (Angstroms) | (nm) | Reflectance | Transmittance |
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9488 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

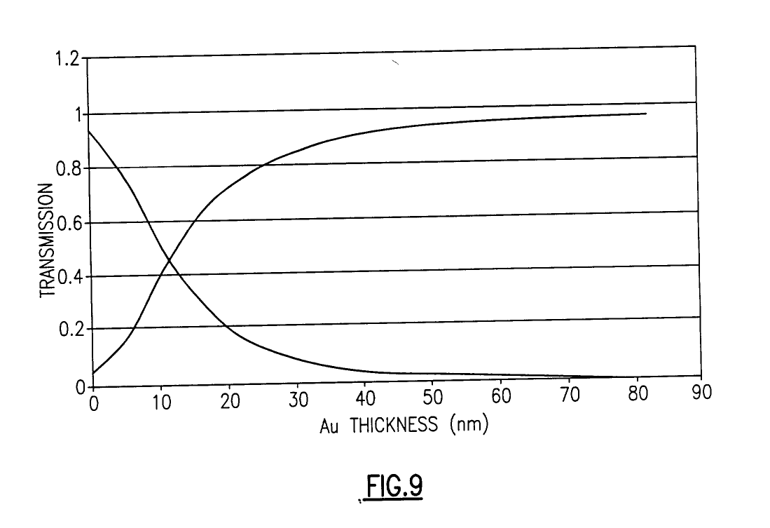

The threshold density for transmission of light through the gold film is approximately 400 Å. In addition to Table 2, FIG. 9 provides a graphical representation of the inverse proportion of the reflective and transmissive nature of the thin semi-reflective layer 143 based upon the thickness of the gold. Reflective and transmissive values used in the graph illustrated in FIG. 9 are absolute values. As shown in FIG. 8, the thinner semi-reflective layer 143 allows a portion of the incident or interrogation beam 152 to penetrate and pass through. The incident or interrogation beam 152 can thus be detected by a top detector 158 as shown in FIG. 10A. At the same time, some of the light is reflected or returned back along the incident path.

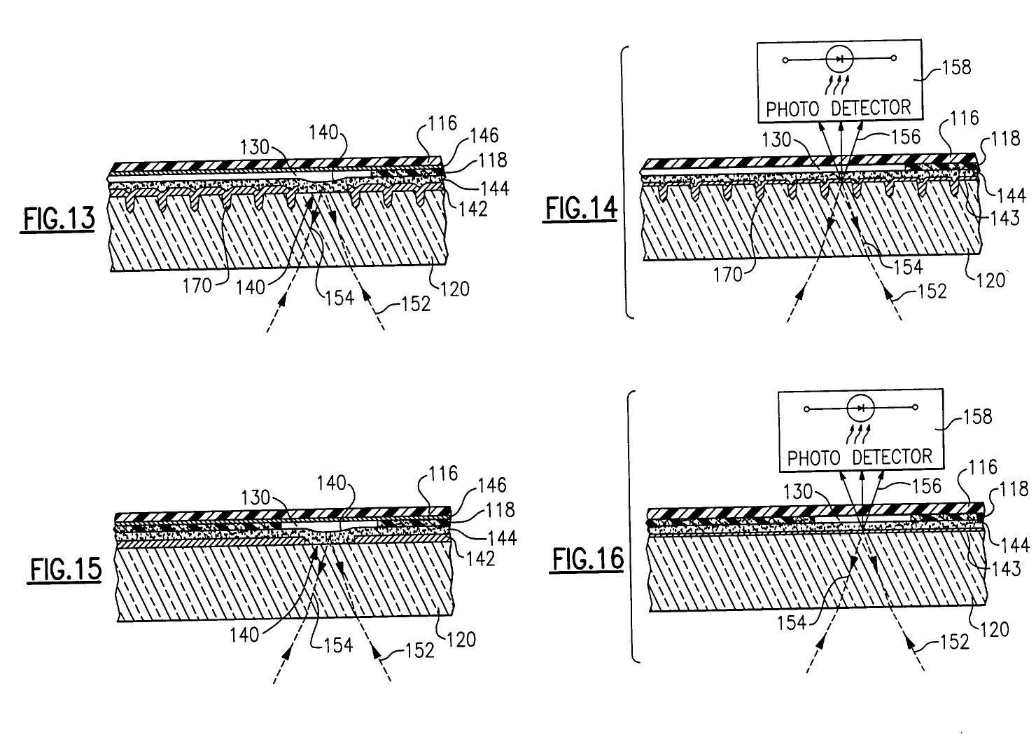

In the case of the reflective optical bio-disc, the return beam 154 carries the information about the biological sample. As discussed above, such information about the biological sample is contained in the return beam essentially only when the incident beam is within the flow channel 130 or target (or capture) zones 140 and thus in contact with the sample. The return beam 154 may also carry information encoded in or on the reflective layer 142 or otherwise encoded in the wobble grooves 170 illustrated in FIGS. 13 and 14. As would be apparent to one of skill in the art, pre-recorded information is contained in the return beam 154 of the reflective disc with target or capture zones, only when the corresponding incident beam is in contact with the reflective layer 142. Such information is not contained in the return beam 154 when the incident beam 152 is in an area where the information bearing reflective layer 142 has been removed or is otherwise absent.

The methods of the present invention may also be readily applied to bio-discs including equi-radial channels such as those disclosed in commonly assigned U.S. Provisional Application Serial No. 60/353,014 entitled “Optical Discs Including Equi-Radial and/or Spiral Analysis Zones and Related Disc Drive Systems and Methods” filed Jan. 29, 2002 which is herein incorporated by reference.

D. System Apparatus

FIG. 10A is a representation in perspective and block diagram illustrating the operation of the system apparatus which includes an optical assembly 148, a light source 150 that produces the incident or interrogation beam 152, a return beam 154, and a transmitted beam 156. In the case of the reflective bio-disc embodiment, the return beam 154 is reflected from the reflective surface 146 of the cap portion 116 of the optical bio-disc 110. In this reflective embodiment of the present optical bio-disc 110, the return beam 154 is detected and analyzed for the presence of signal agents by a bottom detector 157. In the transmissive bio-disc embodiment, the transmitted beam 156 is detected by a top detector 158 and is also analyzed for the presence of signal agents. In the transmissive embodiment, a photo detector may be used as a top detector 158.

FIG. 10A also shows a hardware trigger mechanism that includes the trigger markings 126 on the disc and a trigger detector 160. The hardware triggering mechanism is used in both reflective bio-discs and transmissive bio-discs. The triggering mechanism allows the processor 166 to collect data only when the interrogation beam 152 is on a respective target or capture zone 140. Furthermore, in the transmissive bio-disc system, a software trigger may also be used. The software trigger uses the bottom detector to signal the processor 166 to collect data as soon as the interrogation beam 152 hits the edge of a respective target or capture zone 140. FIG. 10A also illustrates a drive motor 162 and a controller 164 for controlling the rotation of the optical bio-disc 110. FIG. 10A further shows the processor 166 and analyzer 168 implemented in the alternative for processing the return beam 154 and transmitted beam 156 associated the transmissive optical bio-disc. In the case of the transmissive optical bio-disc, the transmitted beam 156 carries the information about the biological sample. In this embodiment, there is pre-recorded information on disc. Detector 158 collects the beam.

In another embodiment of the present invention, a split top detector is used to collect the transmitted beam 156. FIG. 10B shows a split detector according to an embodiment of the present invention. Detector 170 has two detector components 172 and 174. The two detector components gather transmitted beam 156 that is refracted by object 186 (e.g. cell) and generate two signals A and B. Object 186 can be an investigational feature such as a biological cell, for example, a red or white blood cell. A differential signal can be obtained by subtracting one signal from the other (i.e. A-B or B-A). When the detector components are over an area that has an object that scatters the incident beam 152, they detect changes in the signal. Each detector sees a change that is opposite to that of the other detector. In other words, when the light bends toward one detector, it sees an increase in signal while the other detector sees a decrease in signal. Because of this property, signal to noise ratio can be increased significantly by generating a signal that is the difference of the signals produced by each of the two detectors. This difference signal has two advantages. First, any noise (optical or electric) in the system that effects both detectors equally is eliminated in the difference signal. Second, objects of interest on the disc that refract light, rather than just absorb it, will cause a large and easily detected change in the difference signal. This aids the task of analysis, which requires isolation signals generated by objects of interest from background noise.

A more thorough discussion of the split detector is presented in commonly owned U.S. Provisional patent application No. 60/355,090 filed Feb. 14, 2002, entitled “Segmented Area Detector for BioDrive and Methods Relating Thereto” and related Provisional Applications of the same title having Serial Nos. 60/335,123; 60/352,649; 60/353,739; and 60/355,090 respectively filed on Oct. 10, 2001; Jan. 28, 2002; Jan. 30, 2002; and Feb. 7, 2002 all of which are hereby incorporated herein by reference. A more thorough discussion of the different types of detector that can be used in conjunction with the present invention is presented in commonly owned U.S. patent application Ser. No. 10/043,688, filed Jan. 10, 2002, entitled “Optical Disc Analysis System Including Related Methods For Biological and Medical Imaging” which is also hereby incorporated herein by reference.

FIGS. 11 to 16 show cross-sectional views of both reflective and transmissive embodiments to illustrate the optical properties of the discs and how detectors are used to collect information-carrying beams from the disc.

With reference now more particularly to FIG. 11, there is shown a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As shown in FIG. 11, the target zone 140 is formed by removing an area or portion of the reflective layer 142 at a desired location or, alternatively, by masking the desired area prior to applying the reflective layer 142. As further illustrated in FIG. 11, the plastic channel member 118 is applied over the active layer 144. FIG. 11 also shows the cap portion 116 and the reflective surface 146 associated therewith. Thus when the cap portion 116 is applied to the plastic channel member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

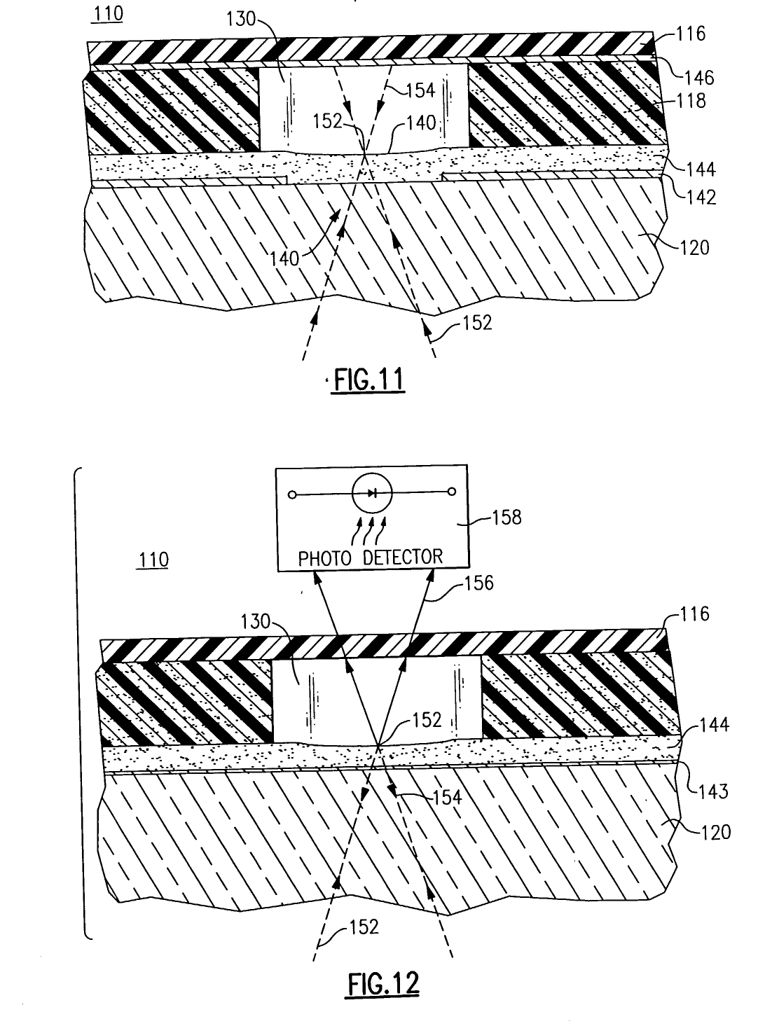

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100 to 300 Angstroms thick and preferably does not exceed 400 Angstroms. This thin semi-reflective layer 143 allows a portion of the incident or interrogation beam 152 from the light source 150, FIG. 10A, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a defined target zone 140 may or may not be present. Target zone 140 may be created by direct markings made on the thin semi-reflective layer 143 on the substrate 120. These marking may be done using silk screening or any equivalent method. In the alternative embodiment where no physical indicia are employed to define a target zone, the flow channel 130 in effect is utilized as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove.” A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive or channel member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention, as described in FIG. 12. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light required by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive or channel member 118 is applied over the active layer 144. FIG. 14 also shows the cap portion 116 without a reflective surface 146. Thus, when the cap is applied to the plastic channel member 118 including the desired cutout shapes, the flow channel 130 is thereby formed and a part of the incident beam 152 is allowed to pass therethrough substantially unreflected.

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these views, the incident beam 152 is illustrated initially interacting with the substrate 120 which has refractive properties that change the path of the incident beam as illustrated to provide focusing of the beam 152 on the reflective layer 142 or the thin semi-reflective layer 143.

E. Analog-to-Digital Processing

Whether obtained from the return beam 154 of the reflective disc or the transmitted beam 156 of the transmissive disc, the information about the biological test sample is directed to a processor 166 (see FIG. 10A) for signal processing. This processing involves transformation of the analog signal detected by the bottom detector 157 (reflective disc) or the top detector 158 (transmissive disc) to a discrete digital form.

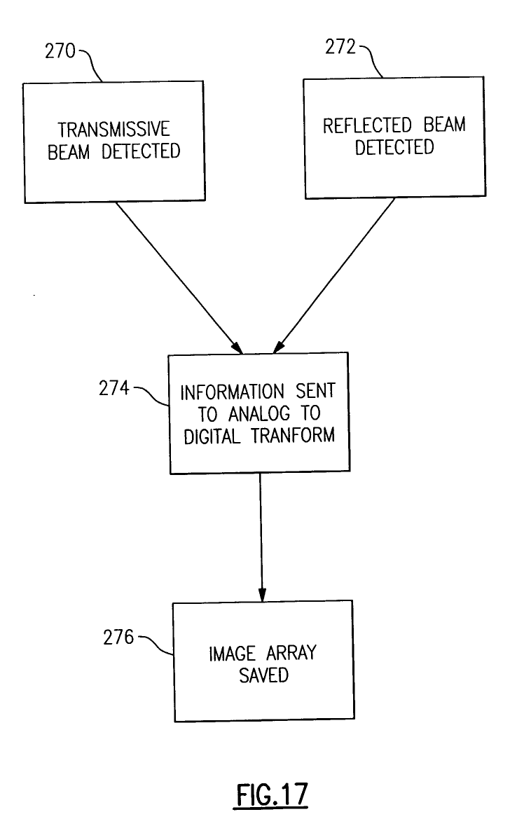

FIG. 17 is a summary flow chart of the information retrieval process related to the apparatus shown in FIG. 10B. In step 270, if the embodiment is a transmissive bio-disc, a transmissive beam carrying the information on the biological sample is detected by detector 158. If the embodiment is a reflective bio-disc, reflected beam 154 is detected by detector 157 in step 272. In either case, in step 274 the information is sent to analog-to-digital transformation. In step 276, the resulting digital data is an array.

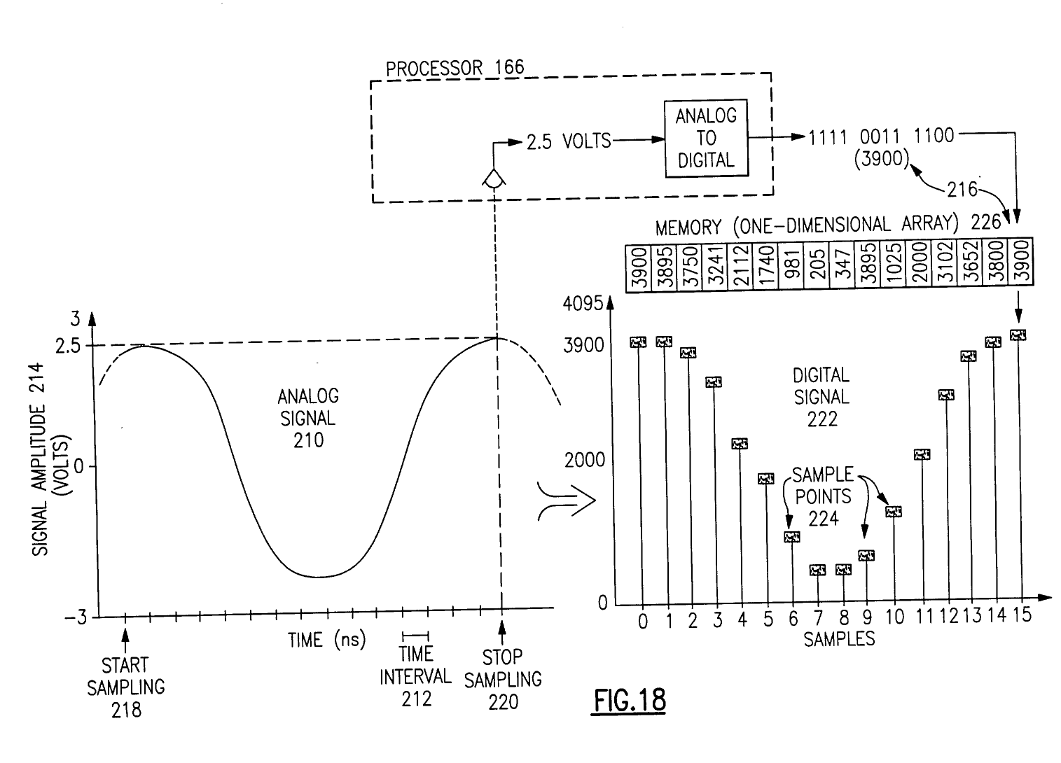

FIG. 18 shows the analog-to-digital transformation performed by processor 166. The transformation involves sampling the analog signal 210 at fixed time intervals 212 and encoding the corresponding instantaneous analog amplitude 214 of the signal as a discrete binary integer 216. Sampling is started at some start time 218 and stopped at some end time 220. The two common values associated with any analog-to-digital conversion process are sampling frequency and bit depth. The sampling frequency, also called the sampling rate, is the number of samples taken per unit time. A higher sampling frequency yields a smaller time interval 212 between consecutive samples, which results in a higher fidelity of the digital signal 222 compared to the original analog signal 210. Bit depth is the number of bits used in each sample point to encode the sampled amplitude 214 of the analog signal 210. The greater the bit depth, the better the binary integer 216 will approximate the original analog amplitude 214. In one embodiment of the present invention, the sampling rate is 8 MHz with a bit depth of 12 bits per sample, allowing an integer sample range of 0 to 4,095 (0 to 2ⁿ−1, where n is the bit depth).

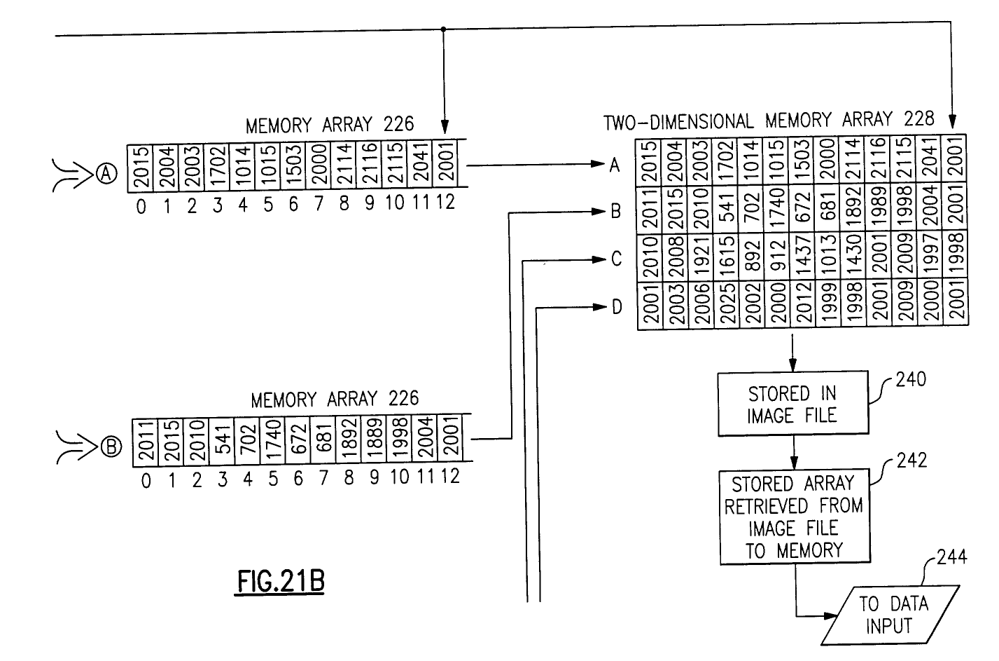

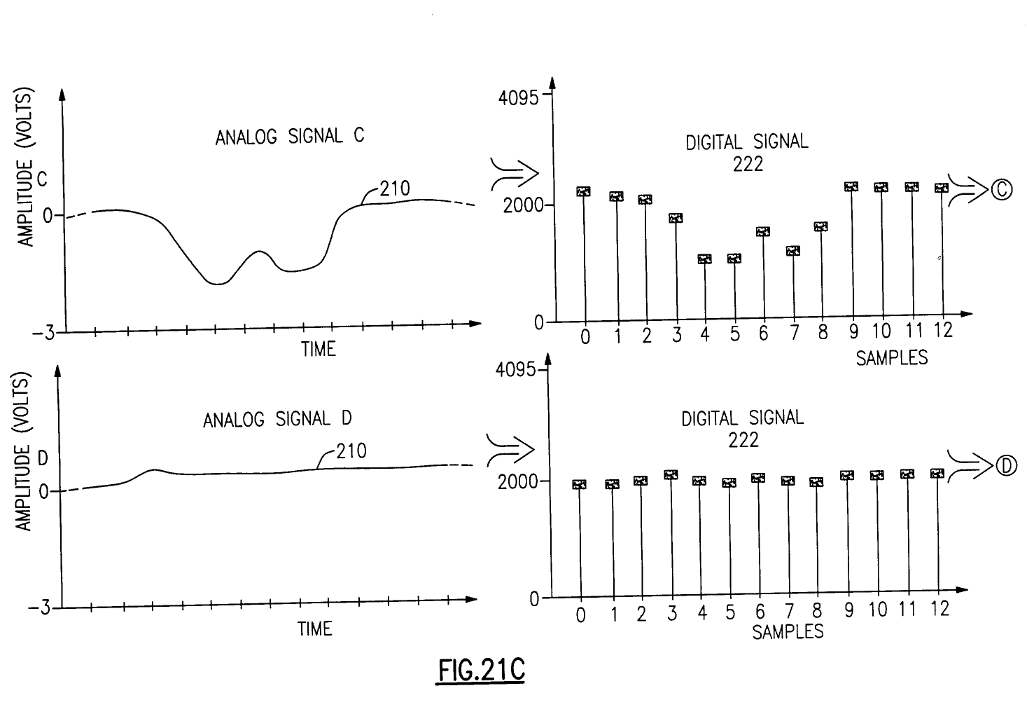

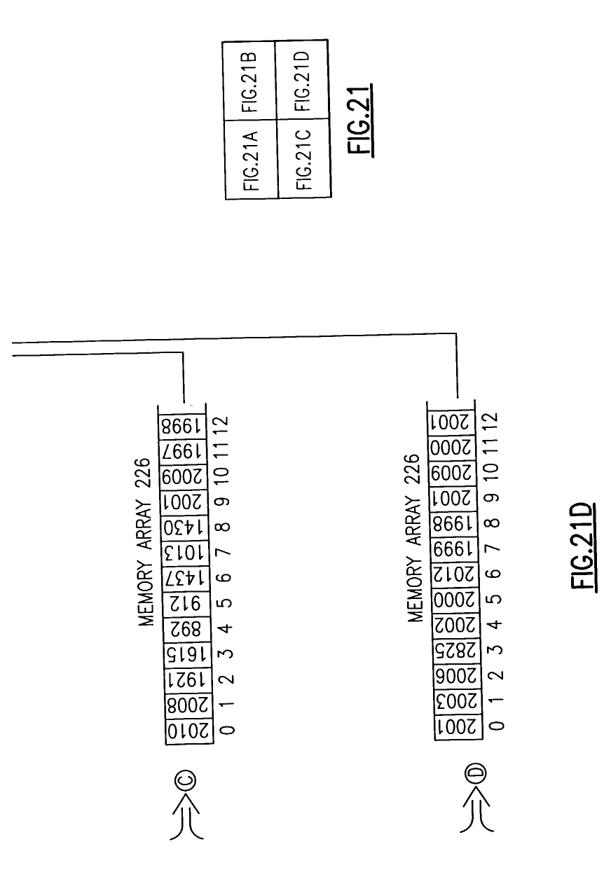

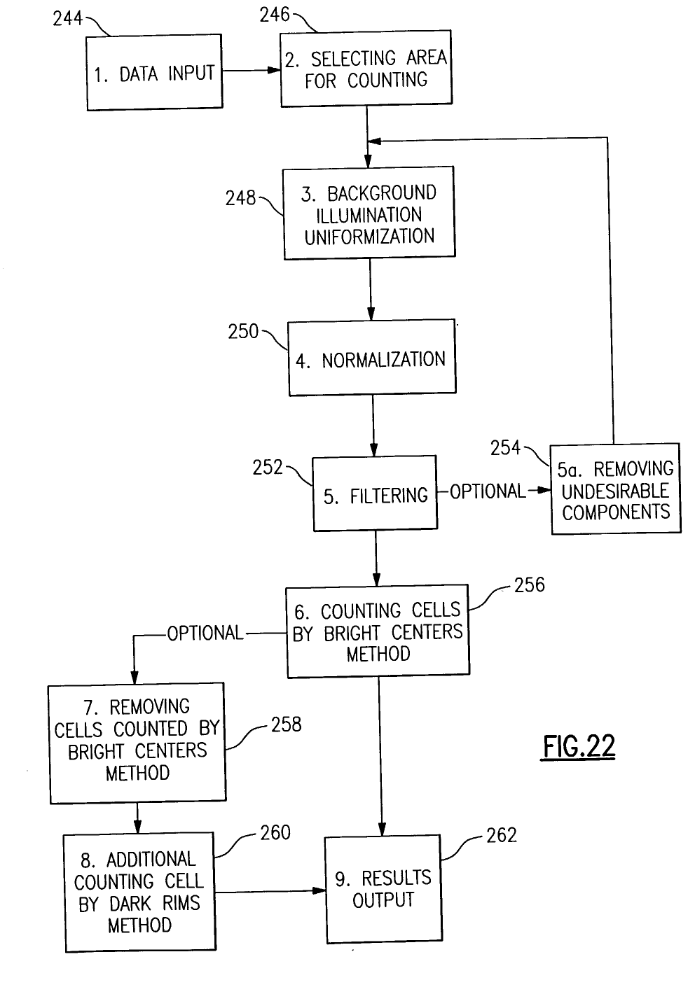

The bit depth and sampling frequency combination can be customized to accommodate the particular accuracy necessary in other embodiments. By way of example and not limitation, it may be desirable to increase sampling frequency in embodiments involving methods for counting beads, which are generally smaller than cells. During the analog-to-digital transformation, each consecutive sample point 224 along the laser path is stored consecutively on disc or in memory as a one-dimensional array 226. Each consecutive track contributes an independent one-dimensional array. All the one dimensional arrays are combined to form a two-dimensional array 228 (shown in FIG. 21B) that is analogous to a common image representation.

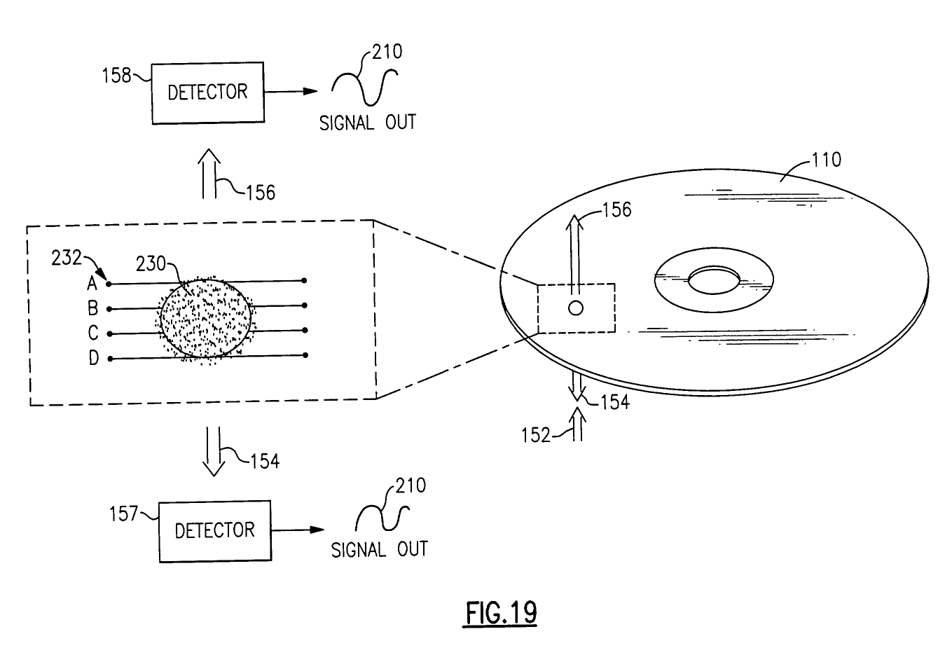

A data collection example is offered here to illustrate further the details involved in data collection from bio-discs. FIG. 19 shows a perspective view of an optical bio-disc 110 of the present invention. The FIG. 19 includes an enlarged detailed perspective view of the section indicated to show a captured white blood cell 230 positioned relative to the tracks 232 of the optical bio-disc. As shown, the interaction of incident beam 152 with white blood cell 230 yields a signal-containing beam, either in the form of a return beam 154 of the reflective disc or a transmitted beam 156 of the transmissive disc, which is detected by either of bottom detector 157 or top detector 158.

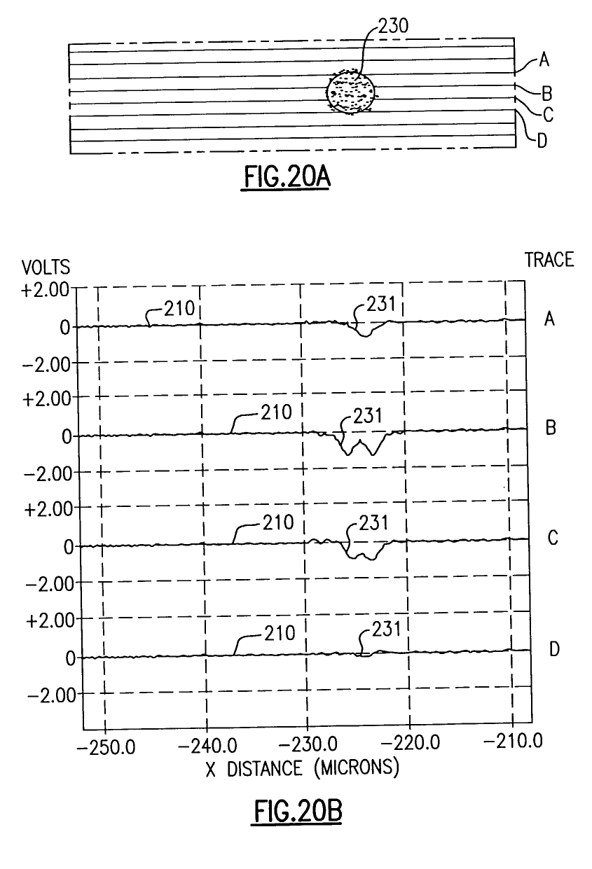

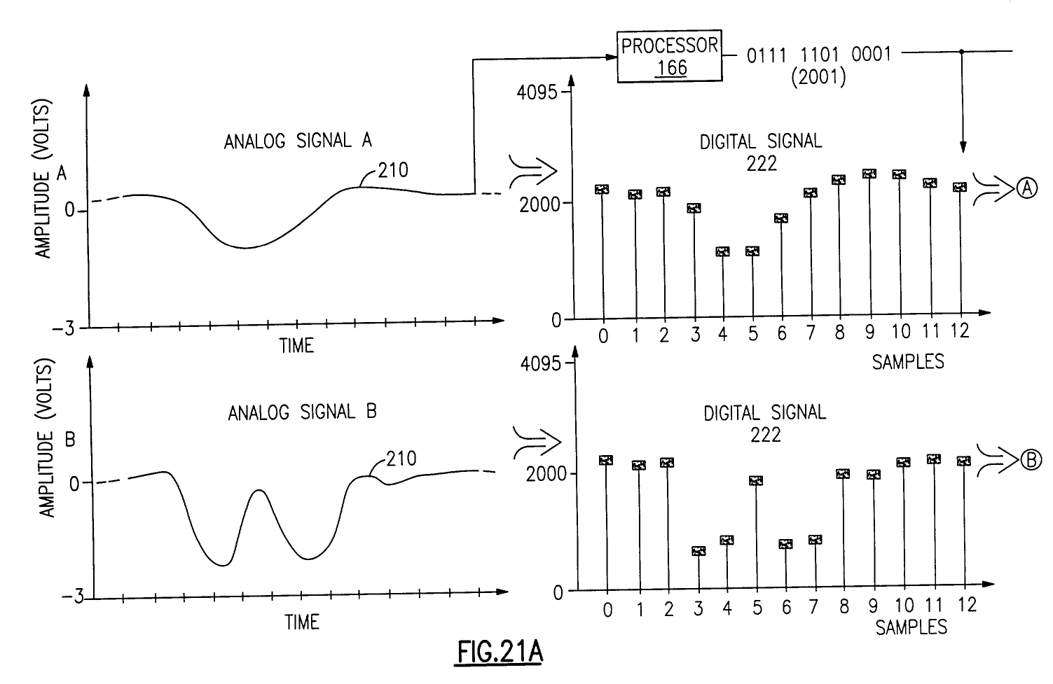

FIGS. 20A, 20B and FIGS. 21A to 21D illustrate how a cell is captured into digital data. In other assays, the investigational feature of interest could instead of a cell, be a bead (bead-based assays), agglutinated matter, precipitate (enzyme reaction), or other biological reporters being of a size that is detectable by the incident beam of the optical system in the present invention. FIG. 20A is a graphical representation of a white blood cell 230 positioned relative to the tracks 232 of an optical bio-disc 110. The cell 230 is located on a disc similar to the disc shown in FIG. 19. FIG. 20B is a series of signature traces derived from the white blood cell 230 of FIG. 20A according to the present invention. FIG. 20B depicts the corresponding traces labeled A, B, C and D. The analog signature traces (signals) 210 are then directed to processor 166 for transformation to a corresponding digital signal 222 (shown in FIGS. 21A-21D). FIG. 20B further reveals that a scan over a white blood cell 230 yields perturbations 231 of the incident beam that can be detected and processed.

FIG. 21 is a graphical representation illustrating the layout relationship among FIGS. 21A, 21B, 21C, and 21D which combine to illustrate how the four traces A, B, C and D from FIG. 20B are converted into a single two-dimensional digital data array 228.

With specific reference now to FIG. 21A, there is shown sampled analog signals 210 from tracks A and B of the optical bio-disc shown in FIG. 20A. Processor 166 then encodes the corresponding instantaneous analog amplitude 214 of the analog signal 210 as a discrete binary integer 216 (FIG. 12). The resulting series of data points is the digital signal 222 that is analogous to the sampled analog signal 210.

Moving now to FIG. 21B, digital signal 222 from tracks A and B (FIG. 21A) is stored as an independent one-dimensional memory array 226. Each consecutive track contributes a corresponding one-dimensional array, which when combined with the previous one-dimensional arrays, yields a two-dimensional array 228 of digital data. The digital data is then stored in memory or on disc as a two-dimensional array 228 of sample points 224 (FIG. 18) that represent the relative intensity of the return beam 154 or transmitted beam 156 (FIG. 19) at a particular point in the sample area. The two-dimensional array is then stored in memory or on disc in the form of a raw file, data file, or image file 240. The data stored in file 240 is then retrieved from memory 242 and used as data input 244 to analyzer 168 (FIG. 10A).

FIG. 21C shows sampled analog signals 210 from tracks C and D of the optical bio-disc shown in FIG. 20A. Processor 166 then encodes the corresponding instantaneous analog amplitude 214 of the analog signal 210 as a discrete binary integer 216 (FIG. 18). The resulting series of data points is the digital signal 222 that is analogous to the sampled analog signal 210.

Referring now to FIG. 21D, digital signal 222 from tracks C and D (FIG. 21 C) is stored as an independent one-dimensional memory array 226. Each consecutive track contributes a corresponding one-dimensional array, which when combined with the previous one-dimensional array, yields a two-dimensional array 228 (FIG. 21B) that is analogous to an image. As above, the digital data is then stored in memory or on disc as a two-dimensional array 228 of sample points 224 (FIG. 18) that represent the relative intensity of the return beam 154 or transmitted beam 156 (FIG. 19) at a particular point in the sample area. The two-dimensional array is then stored in memory or on disc in the form of a raw file, data file, or image file 240. The data stored in the file 240 is then retrieved from memory 242 and used as data input 244 to analyzer 168 (FIG. 10A).

Additional methods and algorithms for capturing data from the optical bio-disc and transforming this data into a two-dimensional array of integers have general broad applicability and have been disclosed in the commonly assigned U.S. Provisional Application Serial No. 60/291,233 entitled “Variable Sampling Control for Rendering Pixelation of Analysis Results in Optical Bio-Disc Assembly and Apparatus Relating Thereto” filed May 16, 2001 which is incorporated herein by reference.

Another embodiment of the invention stores the investigational data in an archive. The archive provides a place where investigational data can be cataloged. Subsequently, groups of data can be analyzed to conduct health trend studies of different population groups. For example, the investigational data can be correlated with patient information to create a catalog of investigational data that can be categorized by the attributes of the patients. Information such as age, sex, race, and blood type, for example, can be used to categorize the investigational data. The archive can take advantage of the features of a searchable relational database. Once such an archive is built, analysis can be conducted on investigational data of a certain category. For example, population health trend studies may be conducted by retrieving investigational data extracted from samples donated by patients from a certain city and analyzing these investigational data. The benefit is that the study may be conducted without the presence of the population itself. Overtime, a historical archive can be built and studies can be conducted on a particular population over a time period and trends over time can be analyzed.

II. Data Analysis

The following sections are directed to the data analysis aspects of the present invention. These are discussed specifically in conjunction with FIGS. 22 to 71D and with general reference to prior FIGS. 1 to 21.

A. Data Collection and Processing

In one embodiment, the investigational data from the bio-disc that is stored in the form of an array of digital data is analyzed for cell counting. In another embodiment, investigational data in other forms are used for analysis. In other assays, the investigational data could contain information for counting beads (bead-based assays), agglutinated matter, precipitate (enzyme reaction), or other biological reporters being of a size that is detectable by the incident beam of the optical system in the present invention. One embodiment of the present invention sends the investigational data in real time to the data analyzer. In another embodiment, the investigational data is stored and later retrieved for analysis. In both embodiments, the computational and processing algorithms of the present invention are stored in analyzer 168 (FIG. 10A) and applied to the input investigational data 244 to produce useful output results 262 (FIG. 22) that may be displayed on the display monitor 114 (FIG. 10A). By way of example of not limitation, the following describes analysis methods for investigational data in the form of digital data arrays. In view of the present disclosure, those of skill in the art may appreciate that the methods of the present invention can be applied to various forms of investigational data beyond the format of digital data arrays.

Moving on now to FIG. 22, there is shown a flow chart presenting a general overview of the steps for data analysis according to the processing methods and computational algorithms of the present invention. A first step of the present processing method involves receipt of the input investigational data 244. As described above, data analysis starts with a two-dimensional array of integers in the range of 0 to 4,095. The next step 246 is selecting an evaluation rectangle of the disc for counting. Once this rectangle is defined, an objective then becomes making an actual count of all white blood cells contained inside the rectangle. This area is termed the “investigational data area”. The implementation of step 246 depends on the configuration of the disc. Two possible disc configurations include discs with windows and discs without windows.

By way of example and not limitation, in embodiments of the invention using discs with windows such as the target or capture zones 140 shown in FIGS. 2 and 4, the software recognizes the windows and crops a section thereof for analysis and counting. In one preferred embodiment, such as that illustrated in FIG. 2, each window (or capture zone) has the shape of a 1×2 mm rectangle with a semicircular section on each end thereof. In this embodiment, the software crops a standard-size evaluation rectangle of 1×2 mm area inside a respective window. In an aspect of this embodiment, the reader may take several consecutive sample values to compare the number of cells in several different windows.

In embodiments of the invention using a transmissive disc without windows, such as that as shown in FIG. 4, step 246 is performed in one of two different manners. The position of the standard rectangle is chosen either by positioning its center relative to a point with fixed coordinates or by finding calibration dot, which is preferably a spot of dark dye with special characteristics. In the case where a calibration dot is employed, a dye with a desired contrast is deposited in a specific position on the disc with respect to two clusters of cells. The optical disc reader is then directed to skip to the center of one of the clusters of cells and the standard-size (1×2 mm) evaluation rectangle is then centered on the selected cluster. In other assays, the cells might instead be beads (bead-based assays), agglutinated matter, precipitate (enzyme reaction), or other biological reporters having a size that is detectable by the incident beam of the optical system in the present invention.

Besides accommodating both types of discs, step 246 also allows for a user's option. The user may specify a desired sample area shape, such as a rectangular area, by direct interaction with mouse selection or otherwise, for cell counting. In the present embodiment of the software, this involves using the mouse to click and drag a shape over the desired portion of a graphical representation of the investigational data that is displayed on a monitor 114 (FIG. 1). Regardless of the evaluation area selection method, a respective rectangular area is evaluated for counting in the next step 248.

The third step in FIG. 22 is step 248, which is directed to background illumination uniformization. This process corrects possible background uniformity fluctuations caused by some hardware configurations. Background illumination uniformization offsets the intensity level of each sample point such that the overall background, or the portion of the investigational data that is not cells, approaches a plane with an arbitrary background value V_{background}. While V_{background} may be decided in many ways, such as taking the average value over the standard rectangular sample area, V_{background} is set to 2,000 in the present embodiment. The value V at each point P of the selected rectangular sample area is replaced with the number (V_{background}+(V−average value over the neighborhood of P)). If needed, the resulting V maybe truncated to fit the actual possible range of values, which is 0 to 4,095 in a preferred embodiment of the present invention. The dimensions of the neighborhood rectangles are chosen to be sufficiently larger than the size of a cell and sufficiently smaller than the size of the standard rectangle. In other assays, the neighborhood rectangles are chosen for beads (bead-based assays), agglutinated matter, precipitate (enzyme reaction), or other biological reporters being of a size that is detectable by the incident beam of the optical system in the present invention.

The next step in the flow chart of FIG. 22 is a normalization step 250. In conducting normalization step 250, a linear transform is performed with the data in the standard rectangular sample area so that the average becomes 2,000 with a standard deviation of 600. If necessary, the values are truncated to fit the range 0 to 4,095. This step 250, as well as the background illumination uniformization step 248, makes the software less sensitive to hardware modifications and tuning. By way of example and not limitation, the signal gain in the detection circuitry, such as top detector 158 (FIG. 13), may change without significantly affecting the resultant cell counts.

As shown in FIG. 22, a filtering step 252 is next performed. For each point P in the standard rectangle, the number of points in the neighborhood of P, with dimensions smaller than indicated in step 248, with values sufficiently distinct from V_{background} is calculated. The number of points calculated should approximate the size of a cell in the investigational data. In other assays, the number of points calculated should approximate the objects of interest including beads (bead-based assays), agglutinated matter, precipitate (enzyme reaction), or other biological reporters having a size that is detectable by the incident beam of the optical system in the present invention. If the number of distinct points found is large enough, the value at P remains as it was; otherwise it is assigned to V_{background}. This filtering operation is performed to remove noise, and in the optimal case only cells remain in the investigational data while the background is uniformly equal V_{background}.

An optional step 254 directed to removing bad components may be performed as indicated in FIG. 22. Defects such as scratches, bubbles, dirt, and other similar irregularities may pass through filtering step 252. These defects may cause cell counting errors either directly or indirectly by affecting the overall distribution in the histogram of the investigational data. This step takes advantage of the fact that these defects are sufficiently larger in size than cells and use the appropriate algorithms to remove them. In other assays, this many be reaily applied to other investigational features such as beads (bead-based assays), agglutinated matter, precipitate (enzyme reaction), or other biological reporters being of a size that is detectable by the incident beam of the optical system in the present invention. Their size in taken into account in determining how best to remove defects. After the optional step 254, steps 248, 250, and 252 are preferably repeated.

The next processing step shown in FIG. 22 is step 256, which is directed to counting cells by bright centers. The counting step 256 consists of several substeps. They are directed to (1) using convolution to make the centers of cells more visible, (2) marking these centers, and (3) performing an actual count of the cells. In some hardware configurations, some cells may appear without bright centers. In these instances, only a dark rim is visible and the following two optional steps 258 and 260 are useful.

Step 258 is directed to removing found cells from the picture. In step 258, the circular region around the center of each found cell is filled with the value 2,000 (the background default) so that the cells with both bright centers and dark rims would not be found twice. Step 260 is directed to counting additional cells by recognizing dark rims. Two transforms are performed on the investigational data after step 258. The goal is to make the dark rims more apparent so cells not counted by the bright center method in step 256 can be counted. In another embodiment, the method of counting cells by dark rims can be used in place of the method of counting cells by bright centers.

After counting step 256, or after counting step 260 when optionally employed, the last step illustrated in FIG. 22 is a results output step 262. The number of cells found in the standard rectangle is displayed on the monitor 114 shown in FIG. 1. Each cell identified is marked with a cross on the displayed optical bio-disc-derived investigational data.

A more detailed description of each step shown in FIG. 22, with corresponding block diagrams outlining the sub-steps in detail, is given below.

Step 1: Data Input

Step 244 retrieves data stored in a two-dimensional array with range from 0 to 4,095. Black segments are filled with value constant zero, while area where light is detected has a range from 1 to 4,095. In consequent data evaluation, zeros are ignored.

Step 2a: Selecting Evaluation Rectangles in Discs with Windows

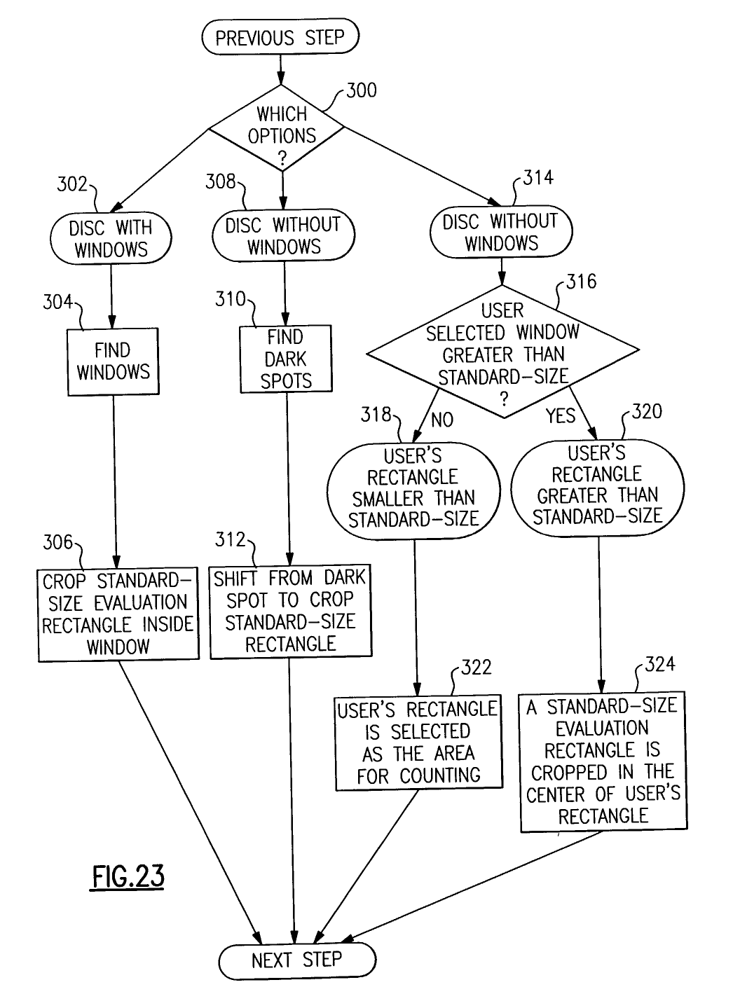

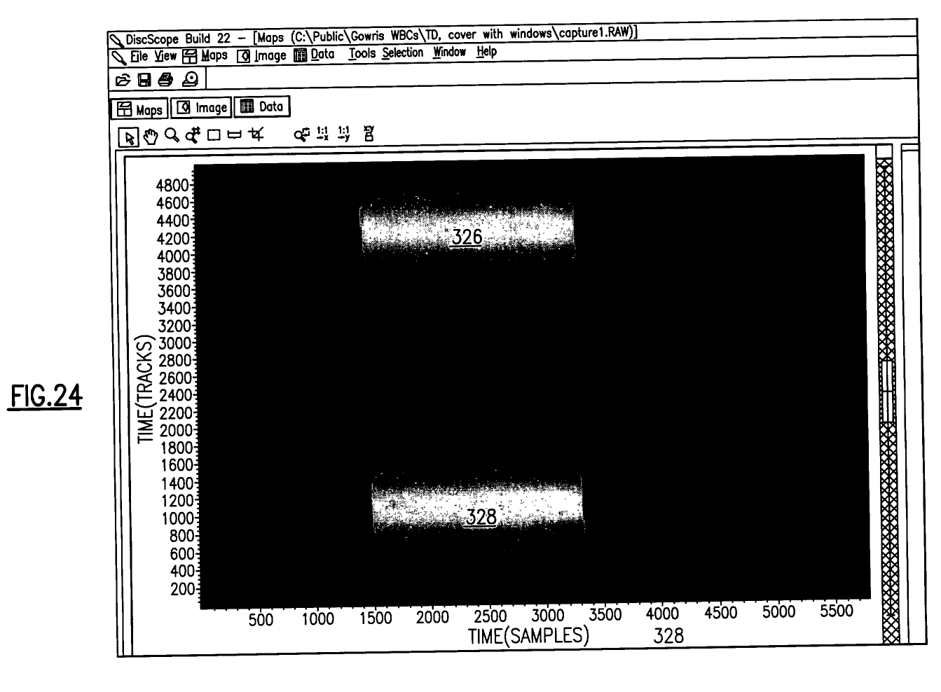

FIG. 23 shows the detailed process of selecting evaluation rectangles (step 246 of FIG. 22). In step 300, the type of selection is determined. The first option 302 involves selecting evaluation rectangles from a bio-disc with physically embedded windows such as target or capture zones 140 shown in FIGS. 2 and 4. FIG. 24 shows a graphical representation of an example investigational data of a disc with windows 326 and 328 as displayed by the software according to an embodiment of the present invention.

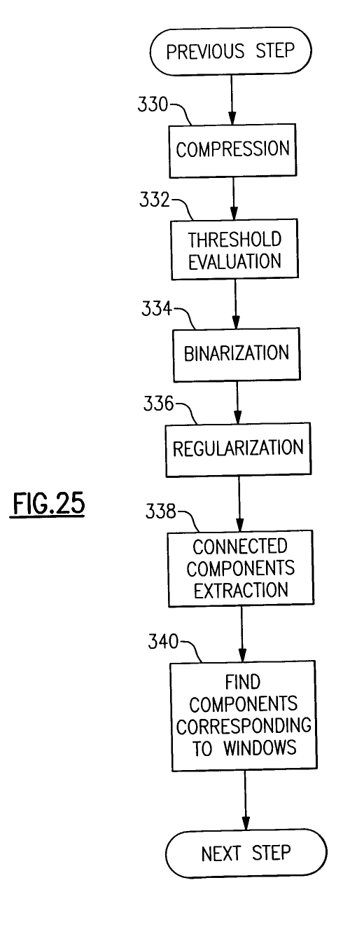

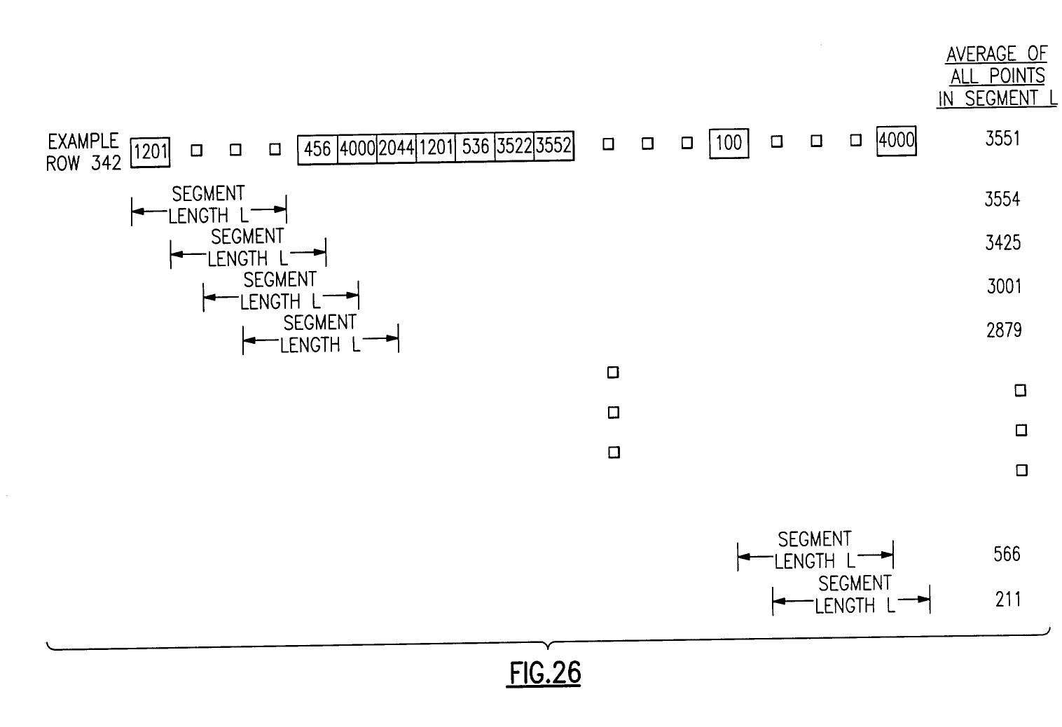

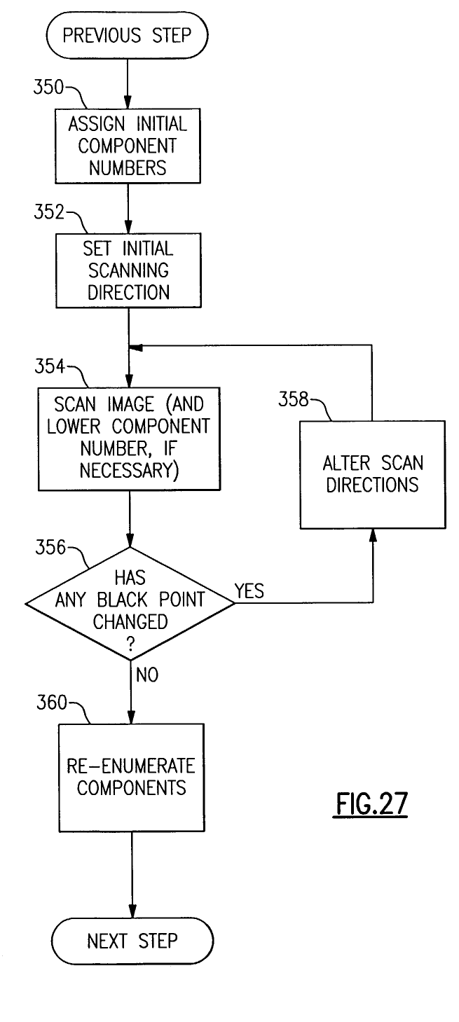

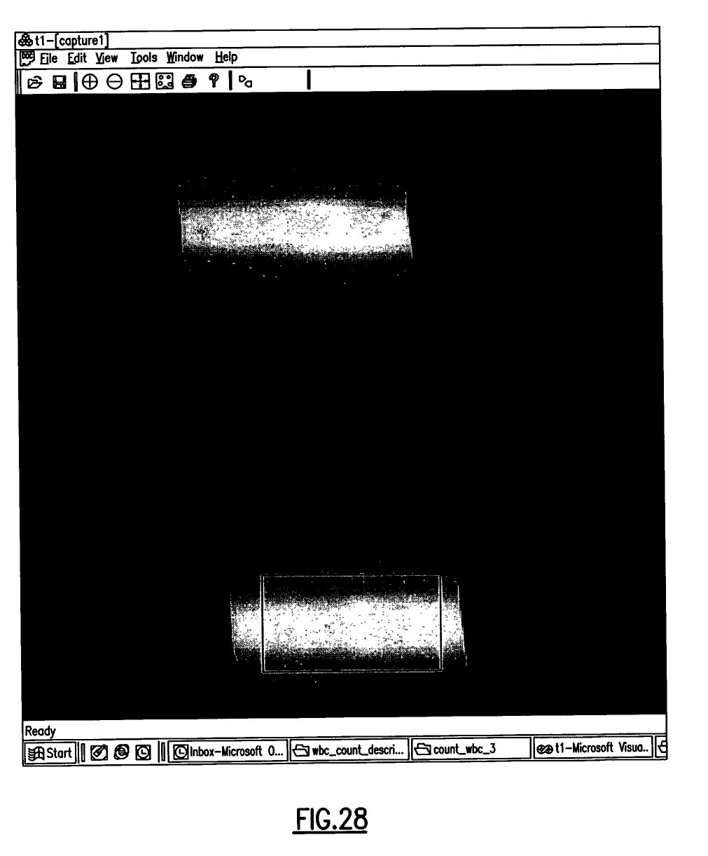

The detailed process of step 304 according to one embodiment is shown in FIG. 25. In step 330, FIG. 25, the investigational data array is compressed in such a way that only every n-th line and every n-th column are considered. Then in step 332, the compressed investigational data is scanned in a row-by-row manner to determine a threshold that will be used in the binarization step. FIG. 26 offers an example for illustration. In example row 342, each cell represents a point on the investigational data and the value in each cell represents the light intensity detected at that point. The scanning begins by selecting all possible segments of length L for each row of the investigational data array. Thus FIG. 26 shows all the possible segments of length L for this example row from the investigational data array. The actual array will have many of such example rows. L is chosen to be slightly less than the width of a window on disc. Then, an average value is calculated for each segment, using all integer values within the segment in question. Because the segment is “sliding” along the row, the process is termed finding “sliding averages”. Once the averages for the all the segments of length L of all the rows are found, the minimum and maximum of the averages are determined. A threshold value T is calculated as (min(averages)+max(averages))/2. This process narrows down the search for windows to segments that cover the window areas. Because window areas are brighter than non-window areas, the averages of these segments will be higher than the threshold value T and can be more easily identified in later steps.

One embodiment speeds the calculation of the average value of a segment L as follows. When calculating the sum a(n) of all values in a segment for n from K+1 till K+L (K+1 being the starting point of the segment), a(K+L) is added to the sum a(n) and a(K) is subtracted from the sum a(n). This is repeated for all values of K. This saves the algorithm from having to add up the sum of all values of a segment all over again each time it moves one unit down the row. This overall process of finding the threshold is repeated for each row until all rows are scanned in this manner.

Returning to FIG. 25, binarization is performed in step 334. In this step, points with value over the threshold value T are declared black while the rest are declared white. So now the investigational data can be treated as if it were a black and white image. Following binarization, regularization is performed on the investigational data in step 336. Regularization consists of two parts: erosion and expansion. Erosion is performed as follows. For an image P, a corresponding image P′ is constructed. A point X′ in P′ is declared white if (1) the corresponding point X in P is white, or (2) any neighbor of X is white. If neither condition is met, then X′ is declared black. P′ is the resulting image of erosion. Expansion operates in an opposite manner. For an image R, a corresponding image R′ is constructed. A point Y′ in R′ is declared black if (1) the corresponding point Y in R is black, or (2) any neighbor of Y is black. If neither condition is met, then Y′ is declared white. R′ is the resulting image of expansion. A composition of several erosions and expansions makes a binary image more regular (single black and single white points disappear).

After regularization is performed, the resulting investigational data is passed to step 338 for the extraction of connected components. In this step, the investigational data is scanned so that connected components are defined. For any given pair of black points in the investigational data, the pair is defined to be in the same component if the two points can be connected by a chain of black points between them. The main purpose of this step is to decompose the investigational data into a collection of connected black components with white spaces separating them.

FIG. 27 shows the sub-steps in the extraction of connected components. The first step, 350, involves assigning initial component numbers. The investigational data is scanned in such a way that the first black point encountered in scanning is assigned a “0”, the next a “1”, and so forth. All the white points are assigned a “−1”. In step 352, the initial scanning direction is set. There are four directions:

(1) “++” denotes top to bottom, left to right,

(2) “+−” denotes top to bottom, right to left,

(3) “−+” denotes bottom to top, left to right, and

(4) “−−” denotes bottom to top, right to left.

Initially the scanning direction is set to “++”, meaning from top to bottom and left to right. In step 354, the investigational data is scanned as follows. Every black point P that has a black neighbor P′ with an assigned number (assigned in step 350) less than the assigned number for P gets the assigned number of P′. For example, if P has a number of 7 and it has a neighbor P′ that has a number of 6, P's new number is 6.

Change determination step 356 follows. The algorithm checks to see if any black point in the investigational data has been assigned a new number by step 354. If so, the scanning direction is altered in step 358. The change of direction follows these rules:

(1) If the current direction is “++”, the new direction is “+−”.

(2) If the current direction is “+−”, the new direction is “−+”.

(3) If the current direction is “−+”, the new direction is “−−”.

(4) If the current direction is “−−”, the new direction is “++”.

The scanning begins again with the new direction in step 354. This cycle continues until a scan can be completed without detecting any changes in component numbers. Alternating the scanning directions upon a component number change reduces the number of scanning passes needed for the process.

After step 356, all points within a connected component should have the same number. In step 360, the points in the components are re-numbered. This step is needed because some of numbers might have disappeared. For example, if there were 20 black points in the initial investigational data, then each point would have received a number from 0 to 19. If after scanning it was found that there were 5 components, 5 of the 20 initial numbers would have disappeared. This would leave the black points in the 5 components with, for example, numbers of 1, 4, 9, 16, and 18. The re-enumeration step would renumber the points in these 5 components from 0 to 4. In general, the enumeration goes from 0 to N−1 for N components in the investigational data. This completes the process of connected component extraction.

Returning to FIG. 25, after the extraction of connected components (step 338) comes the step of finding components that fall within the windows (step 340). The biggest black components meeting certain logical restrictions are selected. The logical restrictions include the number of windows that can be on a disc and the approximate distance between windows. This completes the process of finding windows, which brings the overall rectangle selection process (in FIG. 23) to the next step of cropping standard rectangle inside window (step 306).

In order to find a standard rectangle with (1) the biggest summary illumination and (2) a center at one of the points of component corresponding to window, the area including this component is scanned as follows. First the algorithm scans in the horizontal direction and calculates sliding averages as illustrated in FIG. 26. Then the algorithm scans in the vertical direction and calculates sliding averages. This involves creating segments that run vertically across several rows in the investigational data array. The segments will have the length approximately to that of height of a window. The intersection of the maximum averages from the horizontal sliding averages and vertical sliding averages is declared the center of the evaluation rectangle. This point has the maximum value because it is the brightest spot in the window. The point is selected as the center of the evaluation rectangle. Once the center is defined, in step 312, the 1×2 mm (standard size) evaluation rectangle is created by measuring from the center point. FIG. 28 shows the result of cropping an evaluation rectangle after finding the windows on the software display according to an embodiment of the present invention.

Other techniques for finding the center point include using edge tracing to find the windows, finding the center of gravity (using point values) of a window, and manually selecting a point by inspecting image representing the investigational data.

Step 2b: Selecting Evaluation Rectangles in Discs without Windows

The second option in selecting evaluation rectangles is for bio-discs without windows step 308 in FIG. 23. One embodiment uses the dark spots on the disc to locate the desired location for evaluation rectangles. The algorithm begins in step 310, FIG. 23, with finding the dark spot which serves as indexing markers for indexing the different areas of the samples on disc. The dark spot can be found in the same manner as the process using sliding averages used in finding windows as discussed in conjunction with FIG. 26. Since the targets are now the much smaller dark spots (instead of windows), the segments used in finding the dark spots are much shorter. Their length approximates the size of a dark spot. However, the operating principle of finding sliding averages remains the same. The segments with the lowest of the sliding averages are identified as the dark spots.

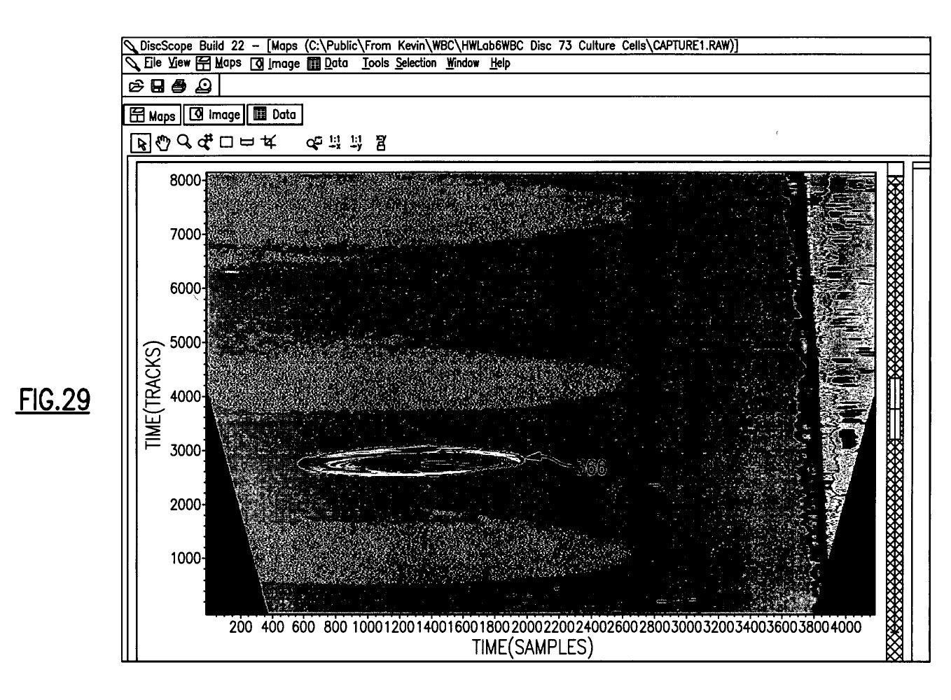

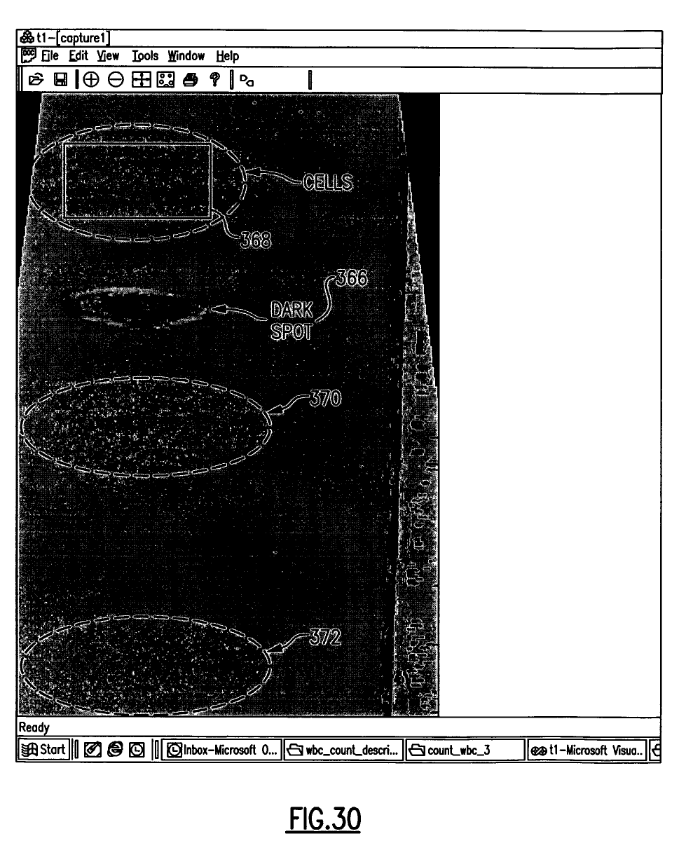

FIG. 29 illustrates an example dark spot 366. In step 312, FIG. 23, once the dark spot is found, the algorithm shifts from the dark spot to create the evaluation rectangle. A standard-size evaluation rectangle is cropped, wherein the rectangle has a center located at a point found by shifting from a pre-determined distance from the found dark spot. FIG. 30 shows an example of an evaluation rectangle 368 cropped out after shifting from a pre-determined distance from the found dark spot 366. Dashed ellipses 368, 370, and 372 identify the other areas of cells. In a preferred embodiment of the invention, the location information of the evaluation rectangle can be embedded on the disc. Instead of finding the dark spot, the system can read location information from the disc to locate an area for placing the evaluation rectangle.

Step 2c: Selecting Evaluation Rectangles including User Selected Option

The third and final option in selecting evaluation rectangles involves input from the user through the software user interface as shown in step 316 of FIG. 23. On screen, an image of the bio-disc created based on the investigational data, is shown to the user and the user selects the evaluation rectangle by defining a rectangle on the image. In step 316, a determination is made as to whether the user-selected rectangle is bigger than the standard size. If not, that means the user's rectangle is smaller than the standard size (step 318, FIG. 23). In this case, the user-selected rectangle is used for counting in step 322. If the user's rectangle is larger than the standard size (step 320, FIG. 23), a standard size evaluation rectangle is cropped from the user-selected rectangle in step 324.

Step 3: Background Illumination Uniformization

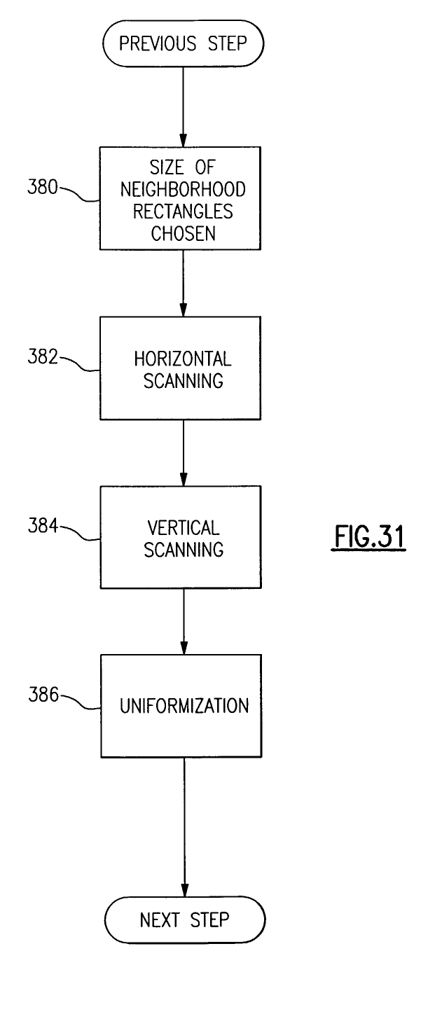

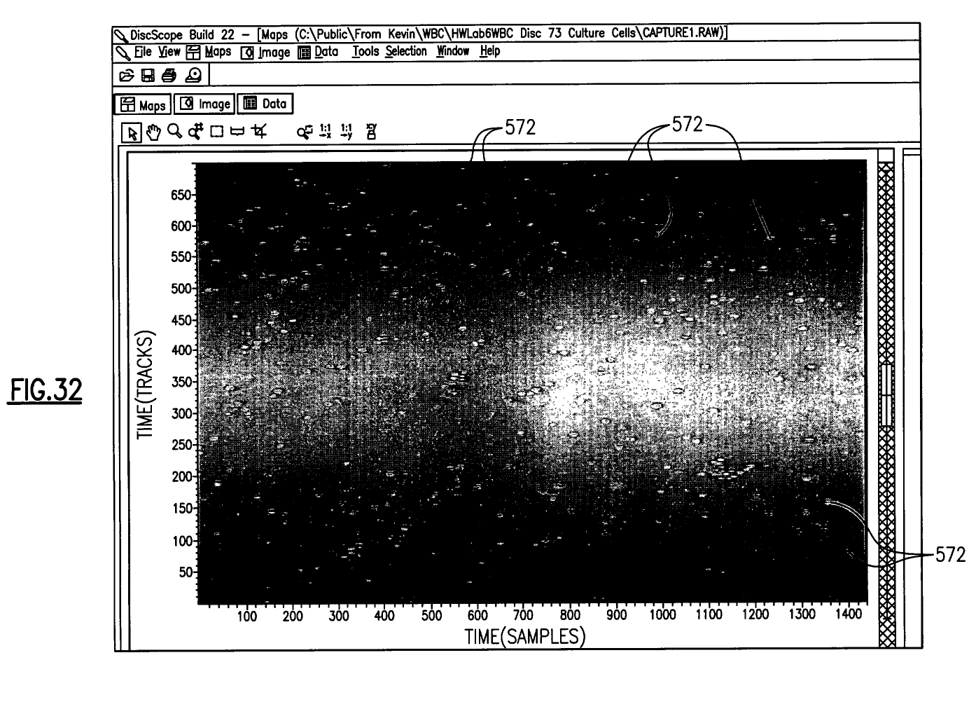

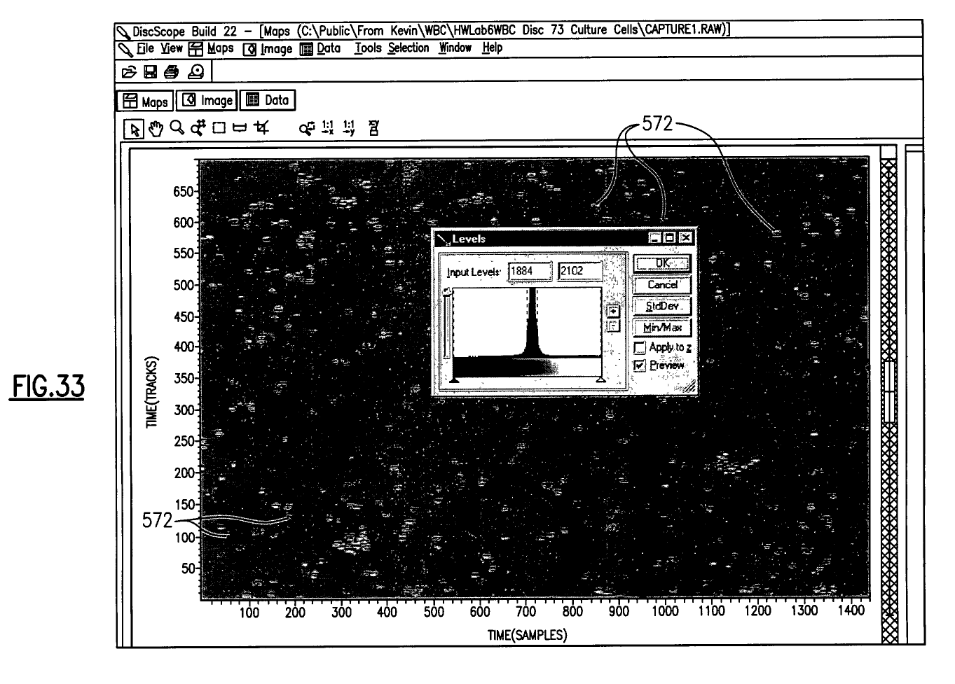

FIG. 31 gives a more detailed illustration of step 248 of FIG. 22. After the evaluation rectangle is selected, background illumination uniformization is performed in the area (termed “investigational data area”) bounded by the evaluation rectangle. The main purpose of this step is to eliminate background noise and thereby make the background more uniform. To accomplish this, background illumination uniformization uses software algorithms to simulate the effect of a gain control in an electrical implementation.

In step 380 of FIG. 31, a standard size for neighborhood rectangles (within the evaluation rectangle) is chosen. Note that a neighborhood rectangle is not to be confused with an evaluation rectangle. The neighborhood rectangle is made to be around a single point of evaluation. Its size is chosen to be sufficiently larger than a cell, but small enough to be affected by the non-uniformity of the background illumination. In other assays, the investigational feature could instead of cells be beads (bead-based assays), agglutinated matter, precipitate (enzyme reaction), or other biological reporters having a size that is detectable by the incident beam of the optical system of the present invention. Thus the size of the neighborhood rectangle is chosen based on the type of assay conducted. The size of neighborhood rectangle determines, for a given point P, how many points near P are evaluated for the process of background illumination uniformization.

Vertical scanning and horizontal scanning are performed in steps 382 and 384, respectively, to calculate an average K for each point in the investigational area.

K is derived in the following manner. First, the vertical scanning is performed. In one embodiment, the vertical averages for all points in the investigational data area are calculated first. The vertical average Kᵥₑᵣₜ for a point (x, y) is the average value of all points in the range from (x, y−dy) to (x, y+dy). In effect, all the columns are scanned in the vertical direction. The term dy is half the height of the neighborhood rectangle, having a size that was determined in step 380. The sliding average calculation technique as described in FIG. 26 is applied here, except now the process goes in a vertical direction.

Once Kᵥₑᵣₜ for all points is found, horizontal scanning is performed as follows. For a point (x, y), the final average K for that point is given by taking an average of all Kᵥₑᵣₜ for points within the range from (x−dx, y) to (x+dx, y). In effect, the rows are scanned in the horizontal direction. The term dx is half the width of the neighborhood rectangle, having a size that was determined in step 380. The overall effect is that, for a particular point P, all the pre-calculated Kᵥₑᵣₜ values that are in the same row as P and within the neighborhood rectangle of P are being averaged to get the final average for P. The pre-calculation of Kᵥₑᵣₜ values reduces the calculation time. Instead of having a calculation time proportional to the size of the investigational data area times the size of the neighborhood rectangle, the calculation time is proportional only to the investigational data area.

With continuing reference to FIG. 31, in step 386, uniformization is performed by reassigning the value V of each point P to V_{background}+(V−K_{neighbor}) where K_{neighbor} is the average value over all points bounded by neighborhood rectangle of P. In one embodiment, the background value V_{background} is set to be the average value over the entire investigational data area. In another embodiment, V_{background} is set to 2,000. If the new value of P is greater than 4,000, then 4,000 is used. If it is less than 1, then 1 is used. Values of P that were previously 0 are replaced with 2,000. After this step, the average value in any large area of the investigational data is approximately 2,000. In other word, the overall background approaches a plane with an arbitrary background value V_{background}. FIG. 32 shows the investigational data as displayed by the software before background illumination uniformization and FIG. 33 shows the investigational data as displayed by the software after background illumination uniformization. Imaged investigational data 572, which is investigational data rendered in an image format, is indicative of data of investigational interest. In both FIGS. 32 and FIG. 33, imaged investigational data 572 mark or correspond to captured cells. Instead of using sliding averages, another embodiment uses Fourier Transform (FT) in the step of background illumination uniformization. In performing the Fourier Transform, the investigational data is first converted to the frequency domain. Then, a part of the spectrum in the frequency domain is removed. This removes part of the background noise generated by electrical noise and other irregularities in the bio-discs as well as in the circuitry. In one embodiment, spectrum with very short or very long wavelengths are removed (frequency is 1 divided by wavelength). The threshold for these removed wavelengths are determined experimentally. Finally, the inverse transform is performed to return the data to the spacial domain.

Step 4: Normalization

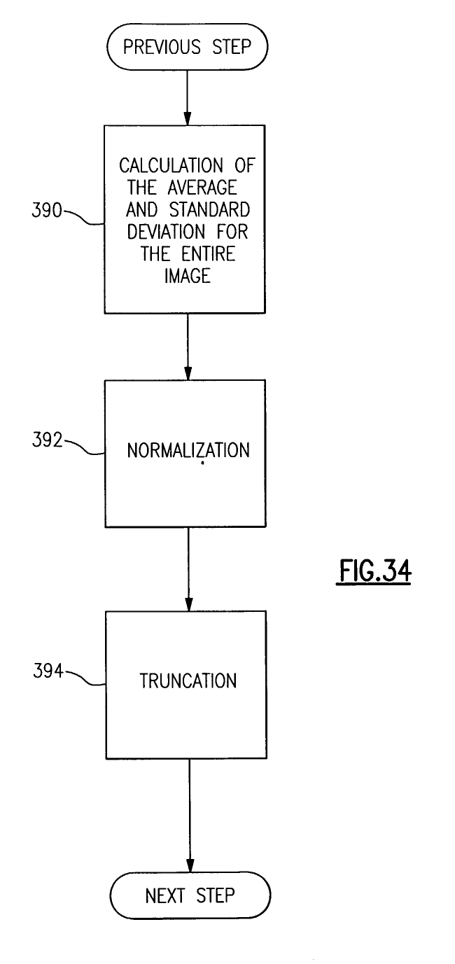

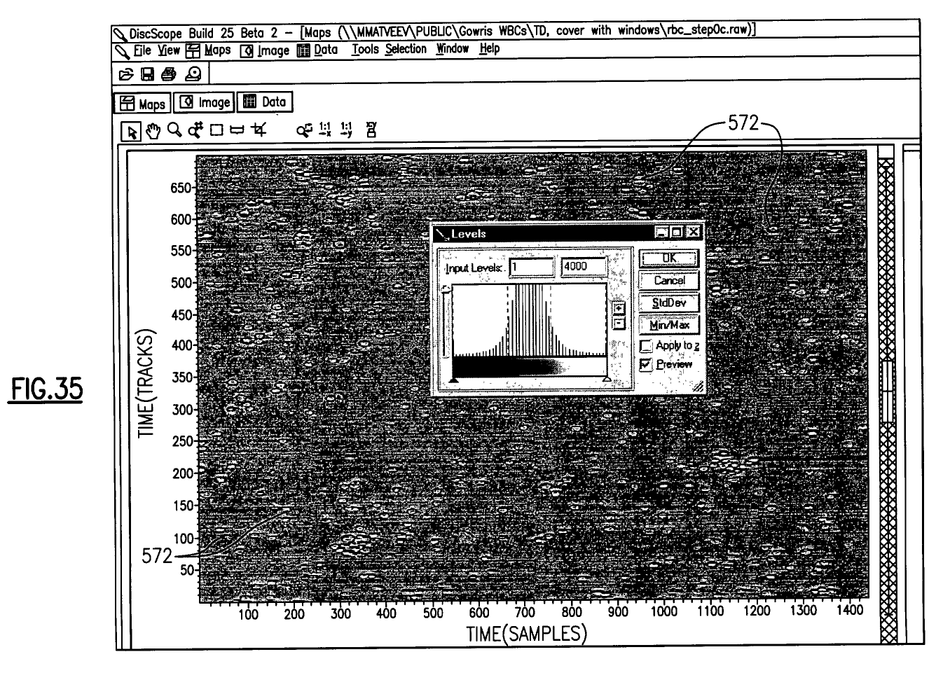

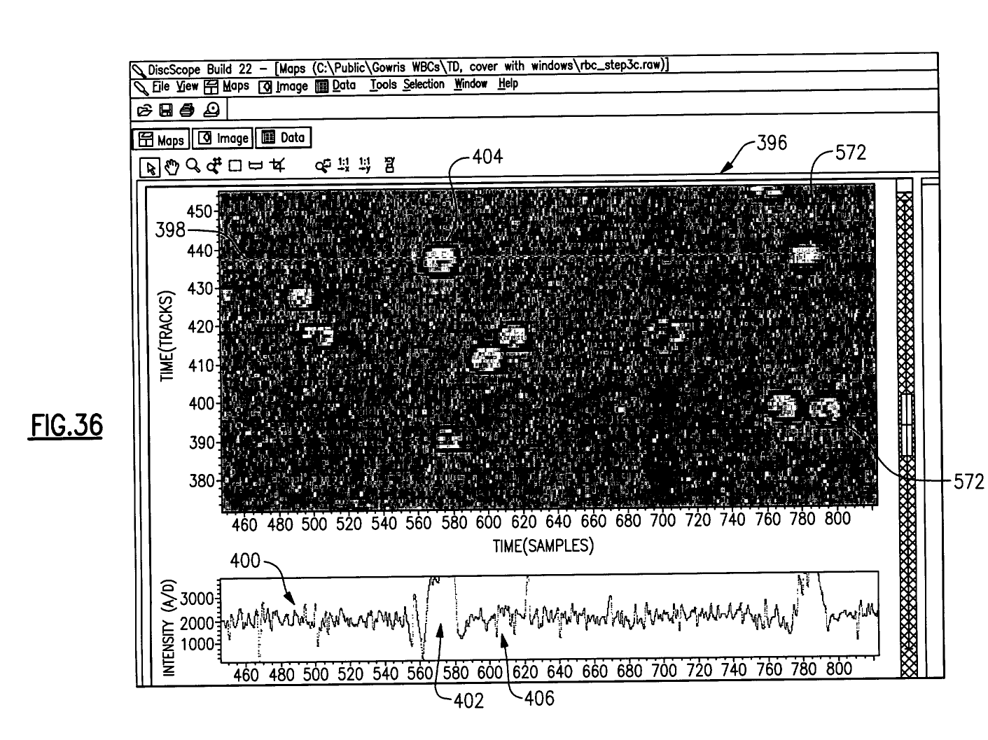

FIG. 34 illustrates in detail the steps involved in step 250 of FIG. 22. As stated herein above in the overview, normalization is needed to make the standard deviation conform to a value of around 600 and the average of the investigational data conform to a value of around 2,000. Normalization also makes the software less sensitive to hardware modifications and tuning. For example, the signal gain in the detection circuitry, such as top detector 158 of FIG. 10A, may change without significantly affecting the resultant cell counts.

To accomplish this, in one embodiment the process begins in step 390 with the calculation of average A and standard deviation S for the resulting investigational data from the last step (background illumination uniformization). Points with value of 0 are ignored. In step 392, FIG. 34, normalization is performed on each point in the investigational data as follows. In one embodiment, for every point P, the value v of P is replace by 2,000+(v−A)*600/(S). The component of (v−A) centers each point while the component (600/S) adjusts the amplitude. The result is added to the background value of 2,000. The value of 600 can be adjusted to get the desired range of amplitude. The value graph 400 of FIG. 36 shows an example of a graph after normalization. Notice that the value graph hovers around 2,000 with the amplitude of the fluctuation staying around 600.

Once the value of P is normalized, truncation (step 394) is performed as follows: 1) if the new value of P is over 4,000, the value is truncated to 4,000; 2) if the new value of P is under 1, the value is truncated to 1.

In one embodiment, the software's graphical user interface displays the histogram of all points in the investigational data to let the user see the process of normalization. FIG. 35 shows a portion of example investigational data as displayed by the software during the step normalization. Imaged investigational data 572 is indicative of data of investigational interest. In FIG. 35, imaged investigational data 572 represent or correspond to captured cells. The input box is asking for a range for normalization. As displayed the value 1 to 4,000 are used. It can be appreciated that any range of values can be used as desired.

FIG. 36 shows the software display after the step of normalization. Top window 396 shows a close-up view of a portion of an example investigational data. Imaged investigational data 572 represent captured cells. Bottom graph 400 shows the corresponding value in the points traced by horizontal dotted line 398 in window 396. Bottom graph 400 shows that the background area of the investigational data is normalized (i.e. a steady value with small noises) whereas areas with cells have noticeable “spikes” in the graph. For example, in bottom graph 400, spike signal 402 corresponds to a specific example cell 404 at that specific location. Spike signal 402 is distinctly different from background noise 406. This eases the process of cell recognition.

Step 5: Filtering

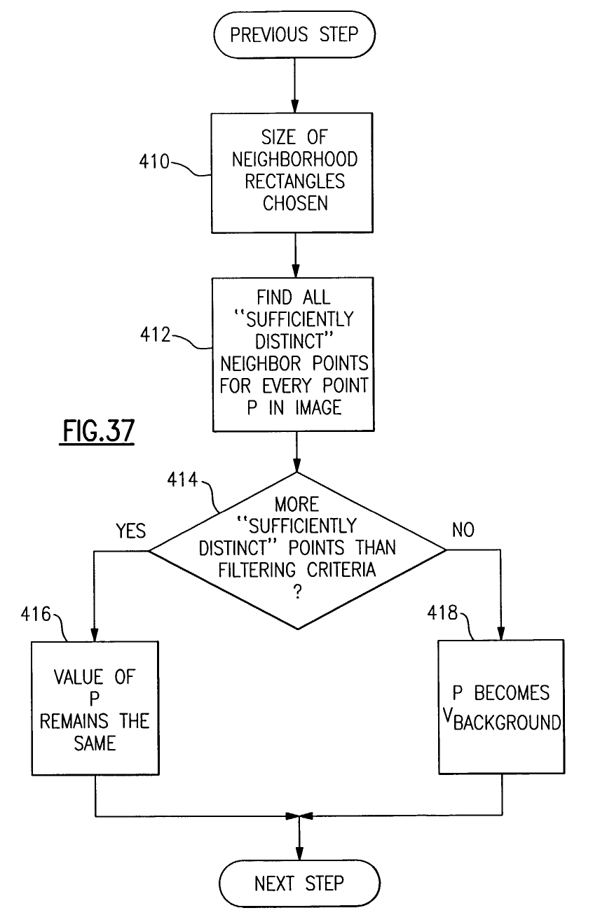

FIG. 37 illustrates in detail the steps involved in step 252 of FIG. 22. In step 410 of FIG. 37, a size for neighborhood rectangles is chosen. These neighborhood rectangles are conceptually similar to the ones used in the step of background illumination uniformization (step 248 of FIG. 22). However, the neighborhood rectangles used in this step are about the size of a cell and smaller than those used in background illumination uniformization. In step 412, for each point P in the investigation data area, the number of points in the neighborhood of P with values “sufficiently distinct” from V_{background} is calculated. In one embodiment, the value of V_{background} is set to 2,000. The determination of how much of a difference (between the point in question and V_{background}) constitutes “sufficiently distinct” is defined by a threshold number. In one embodiment, the threshold number is determined by examining the signal pattern of the investigational data to note the difference between the background value and value of cells (or other objects of interest in the sample). In other assays, the investigational features, rather than being cells, could instead be beads (bead-based assays), agglutinated matter, precipitate (enzyme reaction), or other biological reporters of a size that is detectable by the incident beam of the optical system in the present invention. In other embodiments, the threshold number is generated by a calibration mechanism that determines the background noise and background value based on varying conditions. Such conditions include the reflectivity of the bio-discs, the imbalance of the bio-discs within the bio-disc drives, the rattle, vibration or instability of the bio-discs, electrical noise, metalization of the bio-discs, the types of samples involved (white blood cells or others), and any other conditions requiring compensation or correction by calibration adjustments.

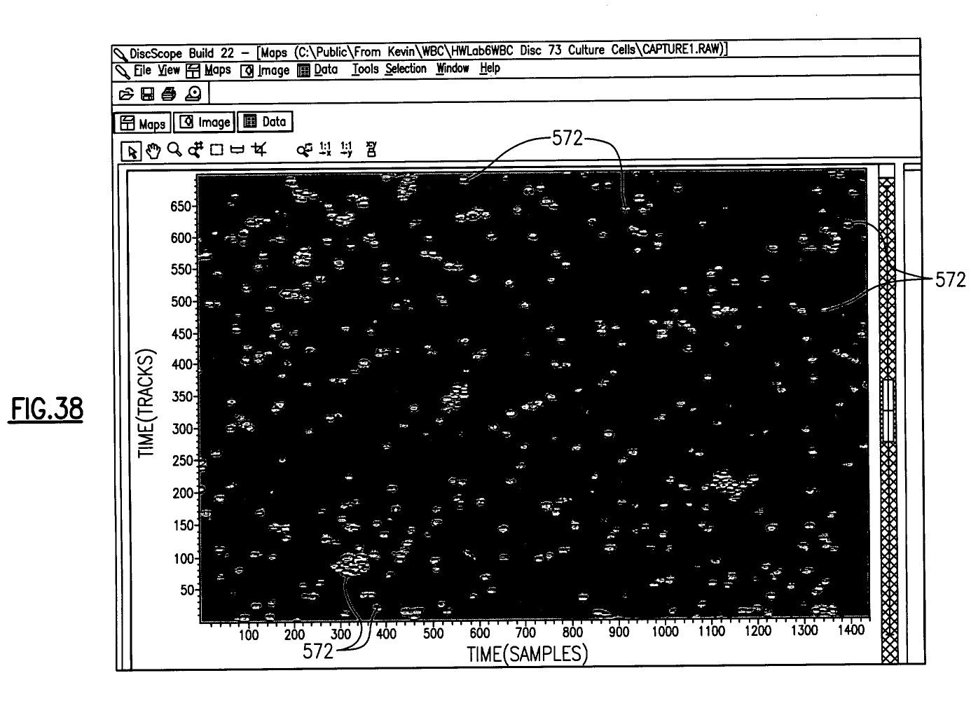

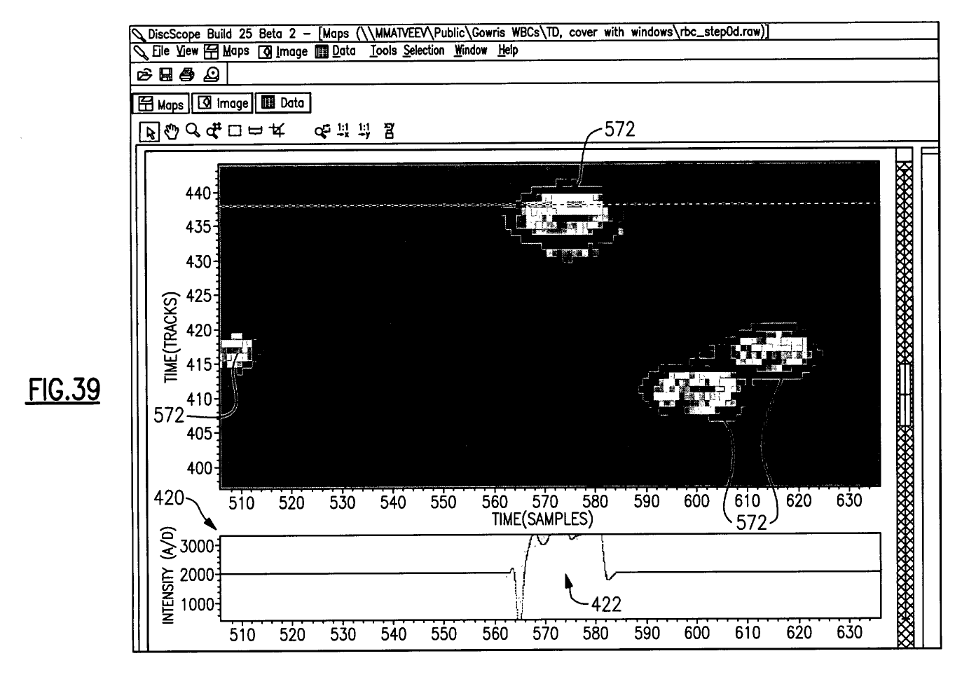

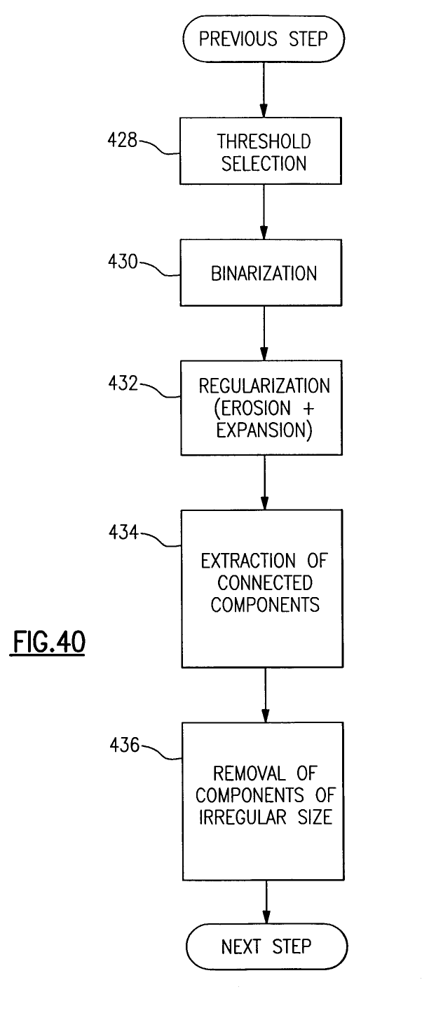

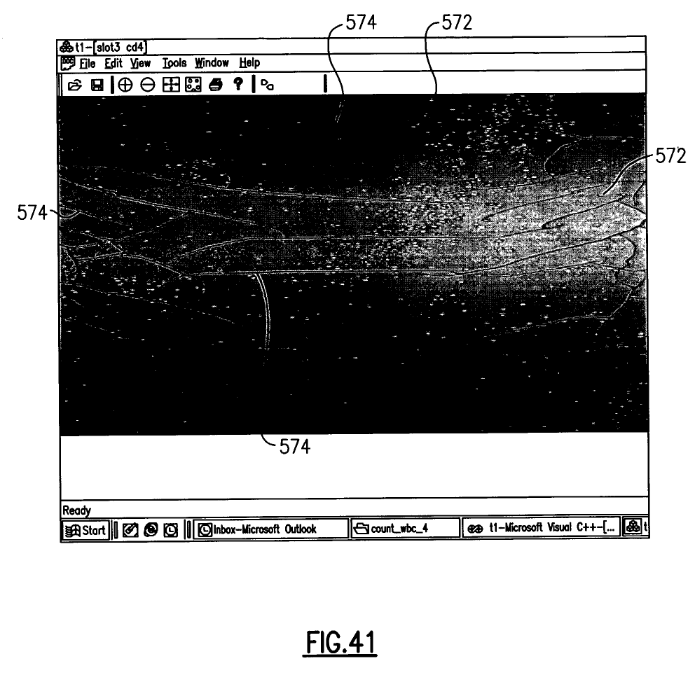

In step 414, the number of “sufficiently distinct” points is tested to see if such number is larger than a pre-determined filtering criteria. If it is, the value of P remains as it is in step 416. Otherwise it is changed to V_{background} (or 2,000) in step 418. The desired effect of this step is to remove noise, so that only cells remain in the investigational data and the background is uniformly equal to V_{background}. FIG. 38 shows an example investigational data as displayed by the software after the filtering step. Imaged investigational data 572 mark or indicate captured cells. Notice that the background has good contrast with the cells in the investigational data. FIG. 39 offers a close-up of a portion of the FIG. 39 investigational data. Imaged investigational data 572 mark and correspond to captured cells. As shown by bottom graph 420 of FIG. 39, the background now has a flat line value and the area with cell is well defined by the spike 422. The improved contrast will help cell counting in the next step.

Step 5a: Remove Undesirable Components

FIG. 40 illustrates in detail the steps involved in step 254 of FIG. 22. This is an optional step designed to eliminate undesirable components such as air bubbles, dirt, and cracks that may interfere with cell counting. The process used here is similar to the process used in finding windows, which is described in connection with FIG. 25. In step 428, FIG. 40, a threshold T is selected. In one embodiment, T is set to V_{background} found in the step of background illumination uniformization. Then in step 430, binarization is performed. Like the binarization step used in window finding (step 334 of FIG. 25), in step 430 points with value over the threshold value T are declared black while the rest are declared white. So now the investigational data can be considered a black and white image and can be represented by 1 bit. In step 432, regularization is performed on the investigational data in the same way as in step 336 of FIG. 25.

After regularization is performed, the resulting investigational data is passed to step 434 for the extraction of connected components. In this step, the investigational data is scanned so that connected components are defined. For any given pair of black points in the investigational data, the pair is defined to be in the same component if the two points can be connected by a chain of black points between them. The main purpose of this step is to decompose the investigational data into a collection of connected black components with white spaces separating them. The connected component extraction process employed here may be the same as the one shown in detail in FIG. 27.

The next step in this removal process is step 436, which is directed to removing components of irregular size. A user-selected size threshold is applied to all the connected components. If a component is smaller or bigger than the size threshold, the entire component is removed from the investigational data. This approach is effective because the size of irregular components (e.g. bubbles, cracks) is usually much greater than the typical cell size. Also, the user can select the threshold according to what type of cells are being counted. In one embodiment, the removal is accomplished by replacing all points with the component with constant value 2,000 (the background value). Preferably, at the completion of all steps in FIG. 40, the investigational data should be sent back to steps 248 through step 252 (FIG. 22) for re-processing.

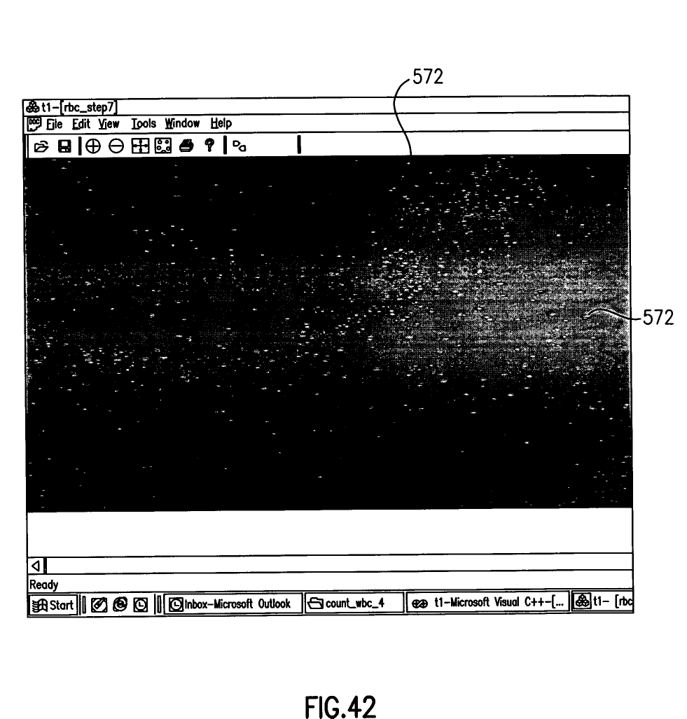

FIG. 41 shows an example of investigational data before the removal of,cracks. Imaged investigational data 572 correspond to captured cells. As shown, cracks 574 are distributed throughout the area. FIG. 42 shows the same investigational data of FIG. 41 after the removal of cracks. Only imaged investigational data 572 marking captured cells remains.

Step 6: Counting Cells by Bright Centers

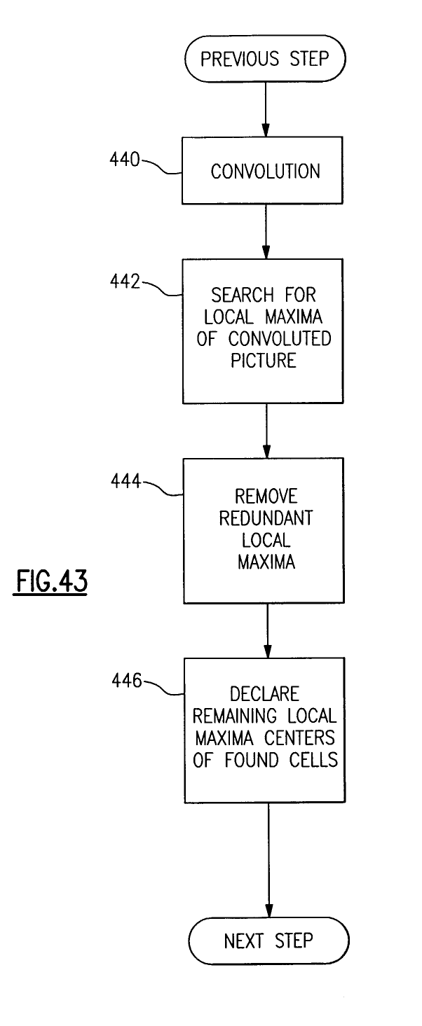

With reference now to FIG. 43, there is shown in detail the steps involved in step 256 of FIG. 22. In step 440, FIG. 43, convolution is performed on the investigational data. During convolution, an auxiliary array representing a convoluted image is formed. Each point P in the convoluted image is the result of integration of the investigational data after filtering in the circular neighborhood of P. As would be appreciated by those skilled in the art, the common convolution method used in image processing involves two functions. The convolution of two functions, f and g : R²−>R is the function:

F(x,y)=ƒog(x,y)=∫∫_{R}_{²}ƒ(x+u,y+v)g(u,v)du dv.

More precisely, in one embodiment of the present invention, the function f that is integrated, is the function:

$$\begin{matrix}f ( x , y ) = h ( x , y ) - 2 , 000 & if h ( x , y ) > 2 , 000 or \\ = 0 & if h ( x , y ) <= 2 , 000\end{matrix}$$

where h(x, y) is function describing the value of the point at x, y from the prior step. Once f(x, y) is established, the convolution is performed with a circular neighborhood indicator function g where:

$$\begin{matrix}g ( u , v ) = 1 & if {u}^{2} + {v}^{2} \leq {r}^{2} or \\ = 0 & otherwise ,\end{matrix}$$

where r is the expected radius of a cell. The convolution integration is:

$$F ( x , y ) = {\int \int }_{{( u - x )}^{2} + {( v - y )}^{2} \leq {r}^{2}} f ( x + u , y + v ) ⅆ u ⅆ v .$$

The integration is replaced by summing values of f in all lattice points (u, v) which are within the circular neighborhood defined by:

(u−x)²+(v−y)²<r².

After convolution, a search for the local maxima is conducted on the convoluted image in step 442, FIG. 43. The convolution step makes the bright centers in the investigational data stand out as local maxima and are thus more easily recognized. Since integer values are used, rounding can create redundant local maxima. To correct this, redundant local maxima that are in the same closed neighborhood are removed in step 444. Then in step 446, all the remaining local maxima are declared centers of cells. In other assays, the cells could instead be beads (bead-based assays), agglutinated matter, precipitate (enzyme reaction), or other biological reporters being of a size that is detectable by the incident beam of the optical system in the present invention. The local maxima are used to find the center of these objects or investigational features targeted in these assays. In other embodiments of the present invention, the counting method takes into account the effect of cell clumping. The maxima that are close to each other are not automatically disregarded. In one embodiment, a local peak is declared the center of cells after a nearby dip. The parameter can be adjusted so that if clumped cells appear on the investigational data, the distance threshold that defines local redundant maxima is adjusted to be smaller. Similarly, the distance threshold can be adjusted for the type of cell that is being counted. For example, since red blood cells have a more consistent size, an assumed cell size can be made to be the distance threshold.

In another embodiment, statistical analysis can be performed on the distribution of cells. Thus an average number of cell per area can be used to estimate the amount of cells in areas where the visibility is low or the cells are clumped so that they may otherwise not be countable. In other assays, the cells could instead be beads (bead-based assays), agglutinated matter, precipitate (enzyme reaction), or other biological reporters having a size that is detectable by the incident beam of the optical system in the present invention. Another embodiment allows the user to re-sample the investigational data area at a higher resolution to perform a more complete and accurate count.

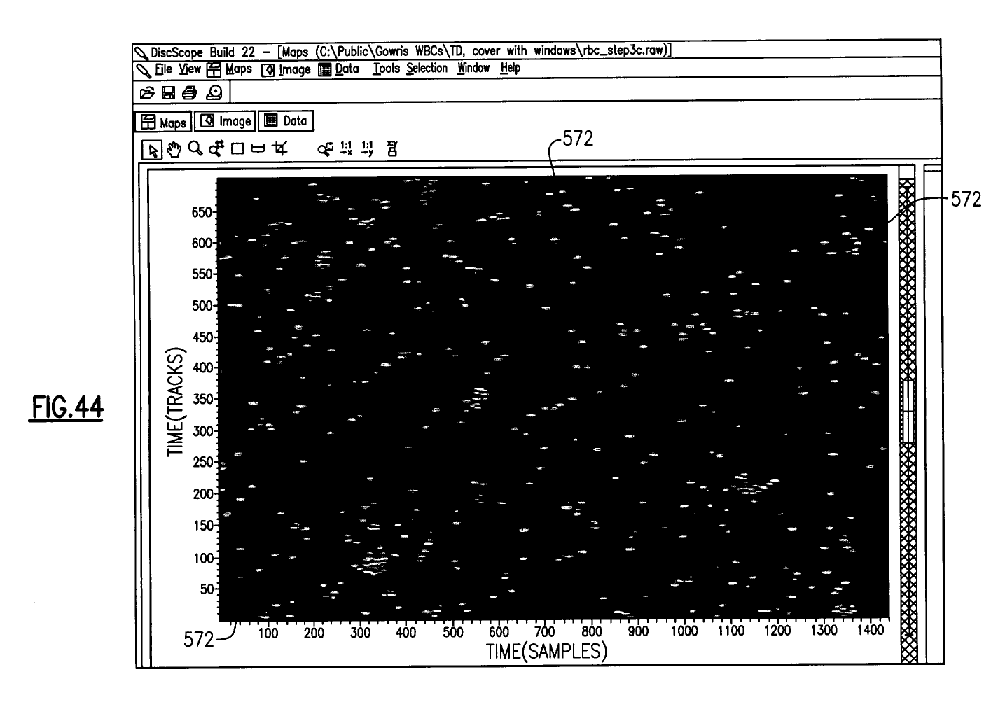

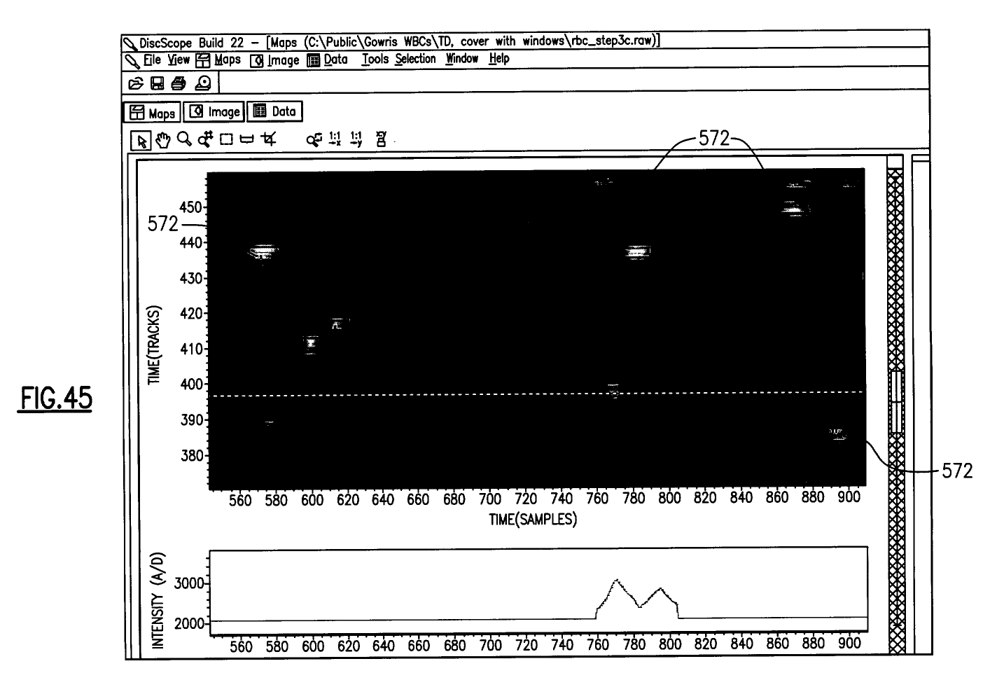

FIG. 44 shows example investigational data filled with cells counted by the bright center method. Imaged investigational data 572 mark captured cells. The steps performed in the bright center method help highlight the cells so they appear brightly against the dark contrast of the background. As shown by the software, they are individually marked and counted. FIG. 45 shows the up-close view and the value trace graph of a portion of the investigational data shown in FIG. 44. Imaged investigational data 572 correspond to captured cells.

Steps 7 and 8: Cell Marking and Additional Counting of Cells by Dark Rims

Steps 7 and 8, FIG. 22, are optional steps that can be performed to improve the accuracy of cell counting. In FIG. 22 they are referenced as steps 258 and 260. These two optional steps may be used to correct under-counting of cells in cases where the hardware configuration may leave cells without bright centers. In other assays, rather applying the present method to cells, it could instead be applied to beads (bead-based assays), agglutinated matter, precipitate (enzyme reaction), or other biological reporters having a size that is detectable by the incident beam of the optical system in the present invention. Alternatively these two steps may be used in place of counting cells by recognizing bright centers.

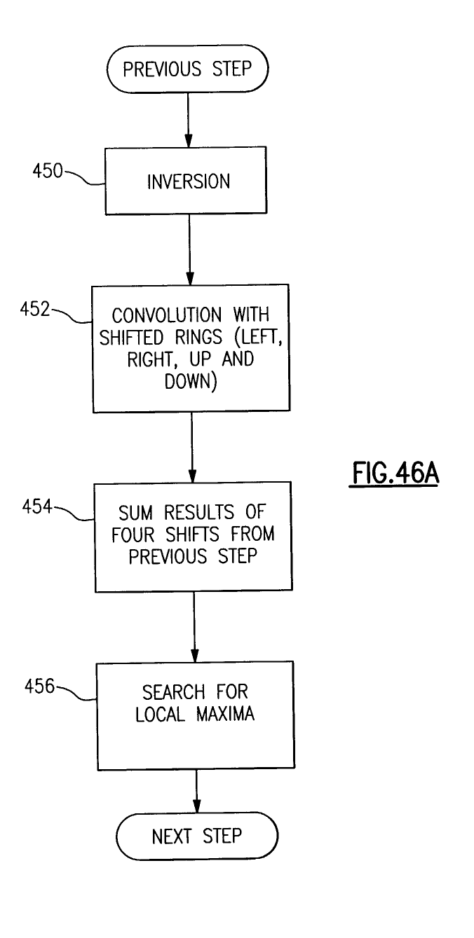

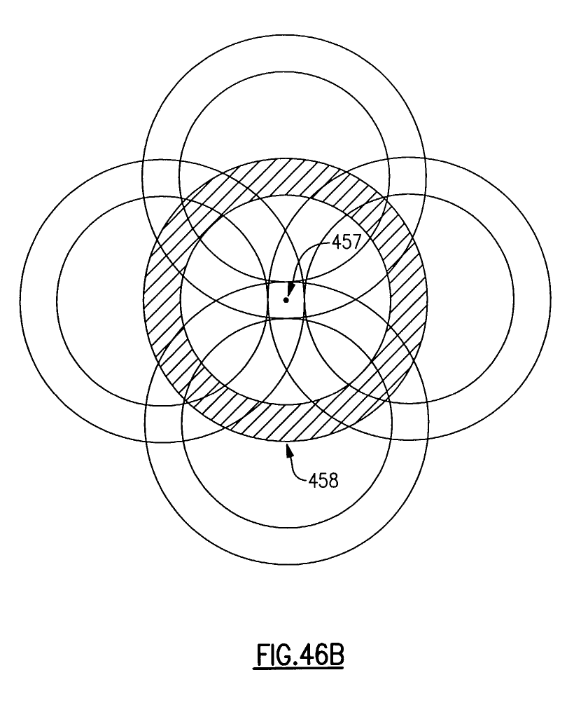

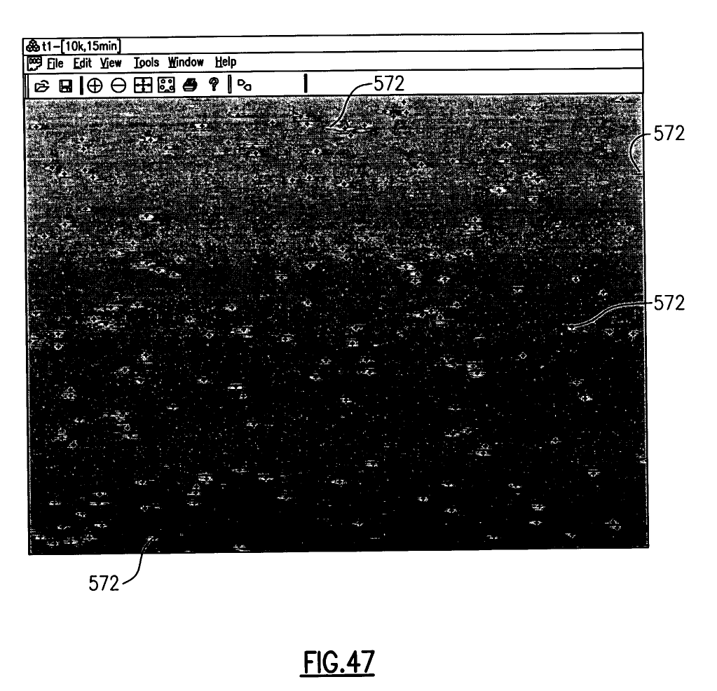

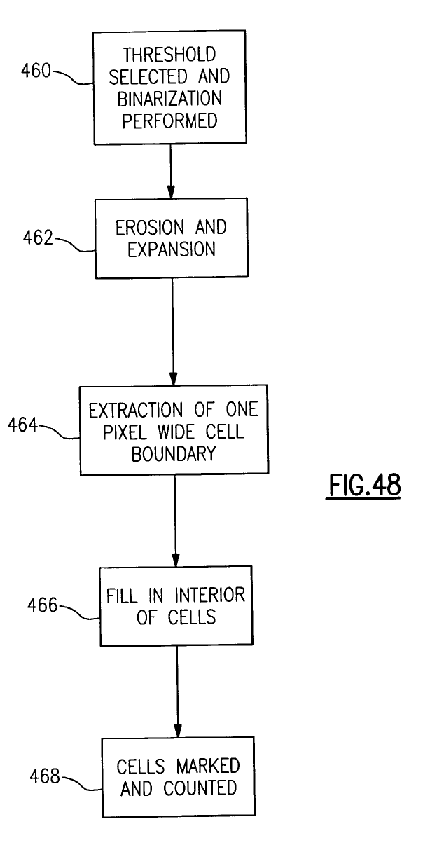

If some cells have been counted by the method of recognizing bright centers, then step 7 is performed to mark these counted cells and remove them from the investigational data. Then counting by recognizing cells with dark rims can proceed. FIG. 46A illustrates in detail the steps involved in step 260 (principal step 8) of FIG. 22. In step 450, inversion is performed on the investigational data. The value v at each point P is replace with 2,000−v. If the resulting value is negative, it is replaced by 0. Expressing inversion in equation form we have the following:

let h(x, y) be investigational data from before, we create f(x, y) to be

$$\begin{matrix}f ( x , y ) = 2 , 000 - h ( x , y ) & if h ( x , y ) < 2 , 000 \\ = 0 & otherwise .\end{matrix}$$

This ensures that the dark rims, which have low data values, will have high values when we perform the convolution. In step 452, convolution with shifted rings is performed. As would be appreciated by those skilled in the art, the common convolution method used in image processing involves two functions. The convolution of two functions, f and g:R²−>R is the function

F(x,y)=ƒog(x,y)=∫∫_{R}_{²}ƒ(x+u,y+v)g(u,v)du dv.

Expressing the convolution in equation form we have:

$$F ( x , y ) = {\int \int }_{g ( u , v )} f ( x + u , y + v ) ⅆ u ⅆ v .$$

The convolution is performed with a circular neighborhood indicator function g where

$$\begin{matrix}g ( u , v ) = 1 & if {u}^{2} + {v}^{2} \leq {r}^{2} or \\ = 0 & otherwise ,\end{matrix}$$

where r is the expected radius of a cell. In this embodiment, g is the indicator function of a ring with inner radius r1 and outer radius r2, where r1 and r2 bound r, the expected radius of a cell. This yields:

$$F ( x , y ) = {\int \int }_{{r1}^{2} \leq {( u - x )}^{2} + {( v - y )}^{2} \leq {r2}^{2}} f ( x + u , y + v ) ⅆ u ⅆ v$$

The integration is replaced by summing values of f in all lattice points (u, v) which are within the ring. To perform the convolution four times, we have four functions: f1(x, y)=f(x+hx,y);

f2(x,y)=f(x−hx, y);

f3(x,y)=f(x,y+hy) and

f4(x,y)=f(x,y−hy),

where hx and hy are specific shifts in the x and y directions. They equal one half of the estimated size of a cell. The four functions mean that the convolution is performed four times with an indicator function of a ring with inner radius r1 and outer radius r2. The values r1 and r2 respectively bound the minimum and maximum of the expected radius of a cell r. The four passes of convolution are performed with the ring shifted in the left, right, up, and down direction with a distance of r. FIG. 46B shows such an example. First, ring 458 is created, bounding the dark rim of the cell. The four-shifted convolution creates four rings. In step 454, FIG. 46A, the results of the four shifts are summed. Returning to FIG. 46B again, we see the summed rings create a local maxima at point 457. Point 457 is then declared to be the local maxima of this cell and is counted. Note that FIG. 46B is an example applied to a cell. In places where the convolution ring does not bound a dark rim of a cell, no maxima would exist. Thus the convolution step locates potential cells by highlighting dark rims of cells. In step 456, FIG. 46A, the counting step goes through the investigational data after convolution to count the local maxima.

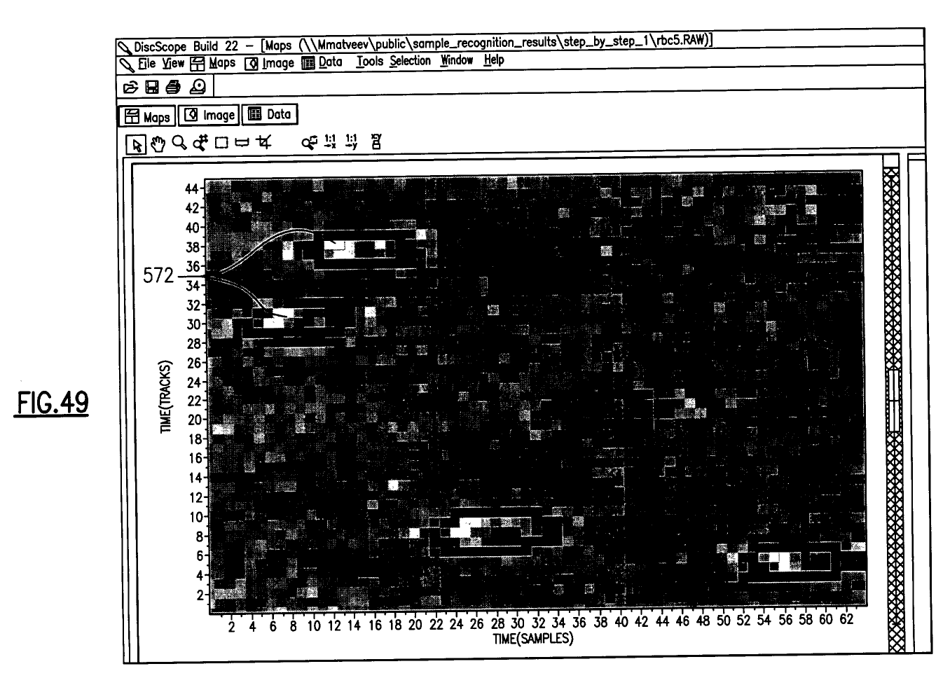

FIG. 47 shows an image of investigational data in which found (counted) cells by this method are marked by crosses. Imaged investigational data 572 correspond to captured cells. Crosses 580 mark counted cells.

Alternatively, the convolution step can be performed in accordance with the equation of the form:

F(x, y)=f1(x, y)+f2(x, y)+f3(x, y)+f4(x, y) if at least three (or alternatively, two) of f1, f2, f3, or f4 are greater than zero, and

F(x, y)=0 otherwise.

Notice that this convolution is performed without shifting the rings if two or three of the functions are greater than zero. Another alternative to the convolution step is to use commonly known smoothing functions to convolve the function f. In yet another alternative that can be applied to both this convolution step and the one used in recognizing bright centers, use of different indicator function g is employed. In one specific embodiment thereof, g can be a Gaussian of the form:

$$g ( u , v ) = {ⅇ}^{- ( \frac{{u}^{2}}{{a}^{2}} + \frac{{v}^{2}}{{b}^{2}} )}$$

or another suitable function for performing convolution for the purpose of highlighting a cell's features.

Step 9: Data Output

In step 9, data are output to appropriate display mechanism. One embodiment of the software has a user interface to display the results of the cell counting for the investigation data areas bounded by evaluation rectangles. Another embodiment displays the image of the investigation data area with each cell marked by a cross as shown in FIG. 47.

B. Red Blood Cell Example

As would be appreciated by those skilled in the art, the various steps and methods of data analysis can be combined in different manners to analyze various types of investigational data. FIG. 48 offers a flow chart illustrating an example of counting red blood cells in investigational data. In step 460, a threshold value is selected and binarization is performed, whereas points with values over the threshold value are declared black and the rest are declared white. This step of binarization separates those points of higher values that usually represent cells and those points of lower values that usually represent the background or background noise.

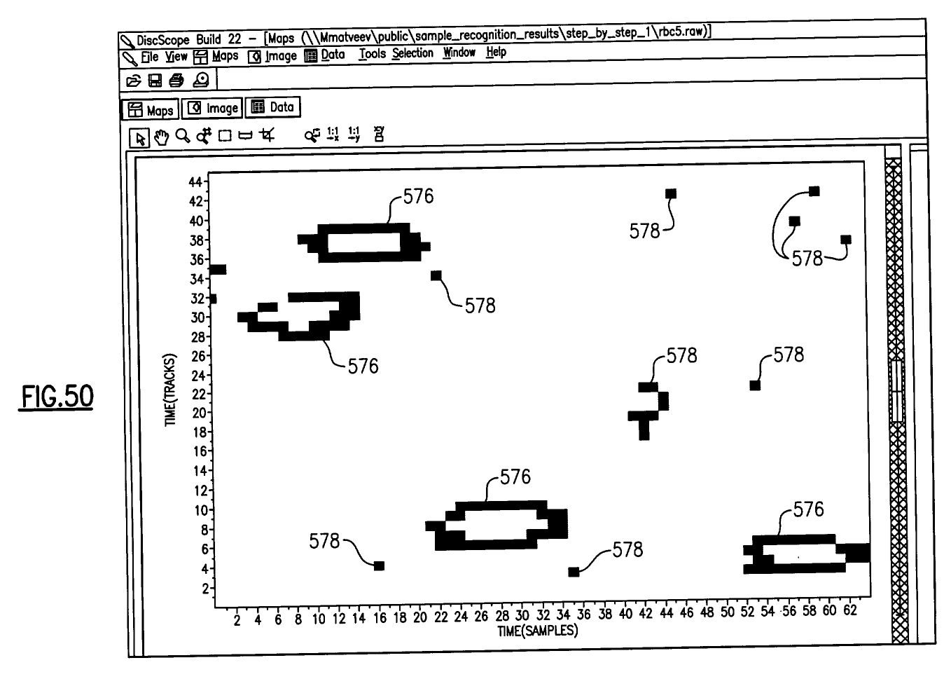

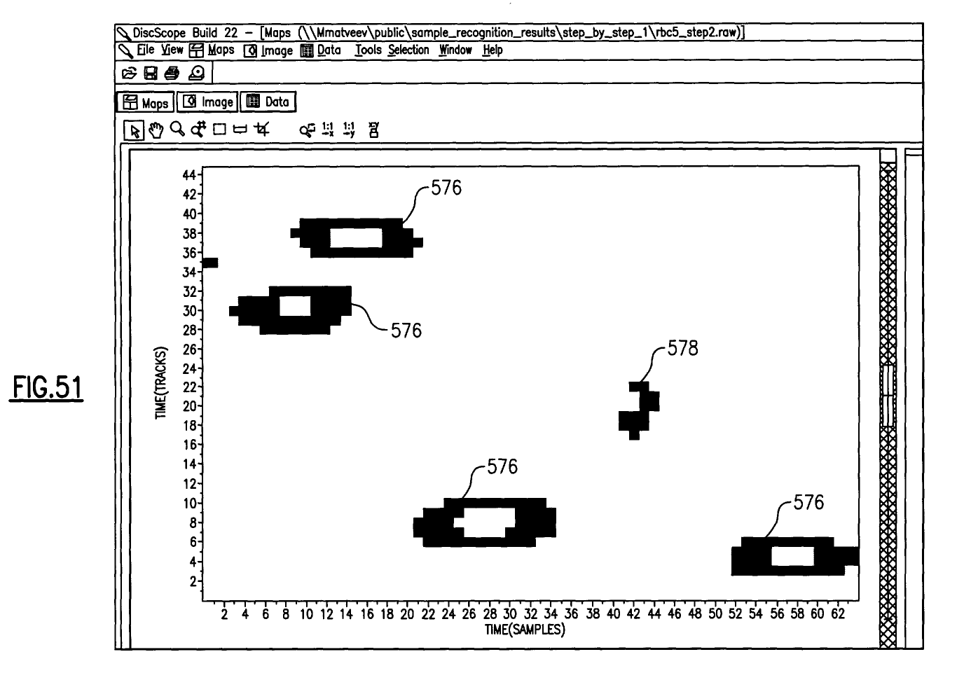

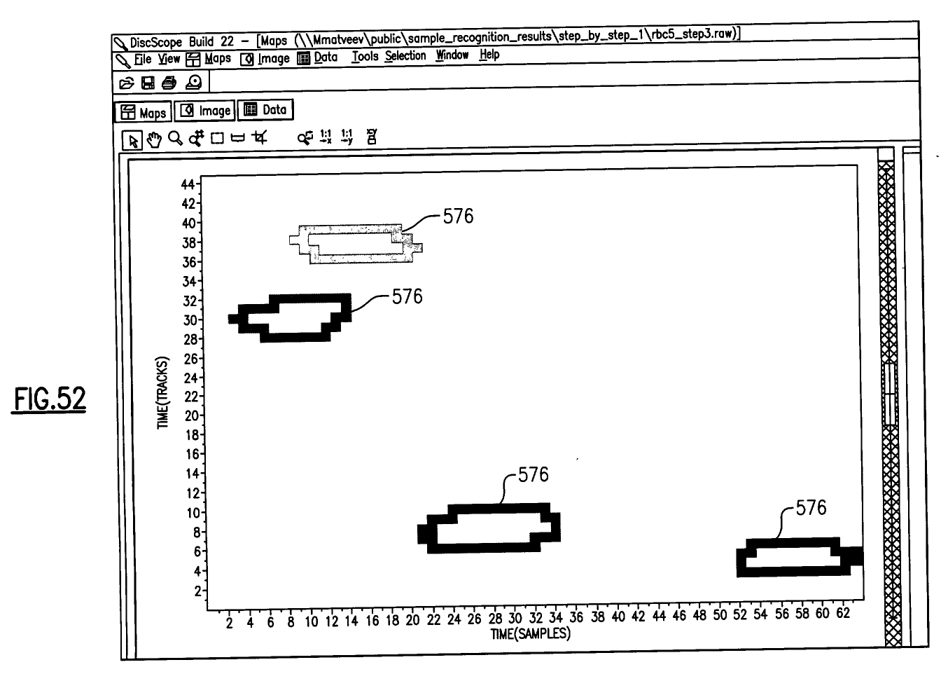

FIG. 49 shows a pictorial representation of the investigational data before step 460 is performed. Imaged investigational data 572 correspond to captured cells. FIG. 50 shows the result of binarization (step 460). Binary imaged data 576 mark or indicate data of investigational interest. In this case, the binary imaged data 576 indicate cells. Non-cell marking binary imaged data 578 point out data that do not represent cells.

Then in step 462, FIG. 48, the two parts that make up regularization, erosion, and expansion are performed to fill in the missing parts of cell boundaries. The goal here is to obtain cell boundaries that clearly mark off individual cells. FIG. 51 shows the result of erosion and expansion (step 462). Binary imaged data 576 mark data of investigational interest. In this case, the binary imaged data 576 mark cells. Non-cell marking binary imaged data 578 point out data that do not represent cells.

In step 464 of FIG. 48, a one-pixel wide cell boundary is extracted for each cell. FIG. 52 shows the result of extracting the one-pixel wide boundary (step 464, FIG. 48). Binary imaged data 576 mark data of investigational interest. In this case, the binary imaged data 576 mark cells. Non-cell marking binary imaged data 578 point out data that do not represent cells. In extracting the one-pixel wide boundary, all black points that have both black and white neighbors are selected to retain their black color. Black points without neighbor points of both colors are converted to white. This yields a rough boundary that is in places several pixels wide. Then the thinning process is applied to eliminate the extra points in the boundary until only one pixel is left in outlining the shapes in the investigational data. The thinning process starts by removing redundant black points from the boundary until only a one pixel wide line marks the boundary of each black area. As shown in FIG. 52, non-cell marking binary imaged data 578, which was in FIG. 51, is now absent.

The thinning process starts with a rough boundary first. The rough boundary consists of all pixels which have neighbor pixels both inside and outside of the cell. After the rough boundary extraction, the data consists of three categories:

(1) pixels inside a cell,

(2) pixels marking the boundary of a cell, and

(3) pixels lying outside of a cell.

The three are related according to three conditions.

(A) all three are connected,

(B) (1) is disconnected from (3) by (2) and finally,

(C) each point in (2) has a neighbor in (1) or (2) but not both.

The thinning process then examines pixels in (2) one-by-one. If a pixel P in (2) has a neighbor in (1) or (3), then a check is performed to see if re-coloring P (e.g. black to white) from (2) to (1) or (3) still preserves conditions (A), (B), and (C). If so, then the re-coloring will be done. This re-coloring is performed until a one-pixel wide boundary is obtained for each cell.

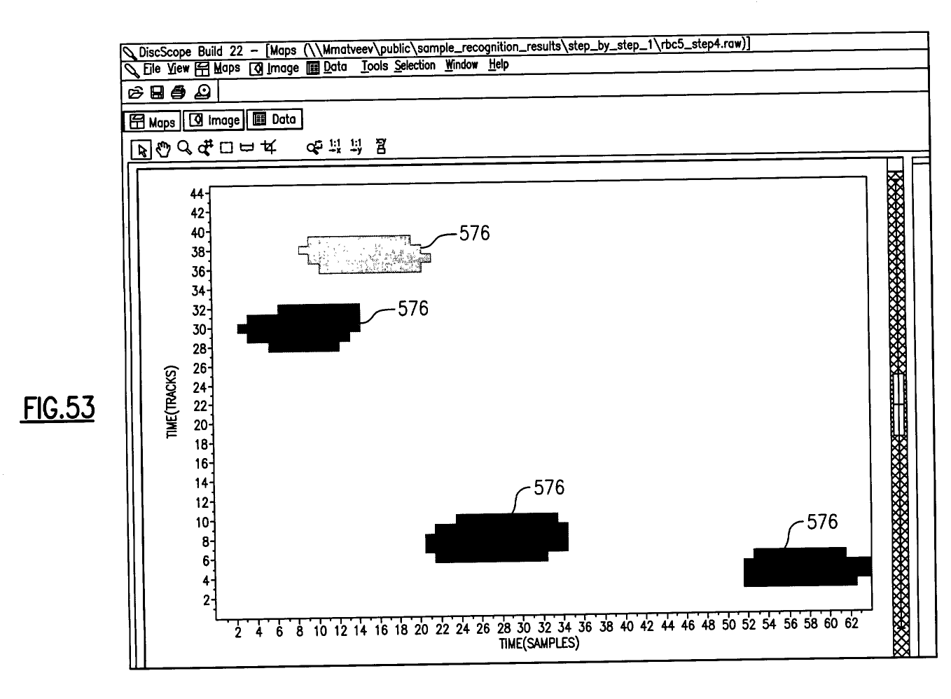

After the one-pixel wide boundary is extracted, the areas defined by the one-pixel wide boundaries are filled in with black points (step 466, FIG. 48). FIG. 53 shows the result of step 466. Binary imaged data 576 mark data of investigational interest. In this case, the binary imaged data 576 mark cells. Non-cell marking binary imaged data 578 point out data that do not represent cells as seen in FIG. 51 for example. Using this set of black and white points as a mask, the original data points are filled in to replace the black points. Thus the cell areas are isolated and are now ready to be analyzed.

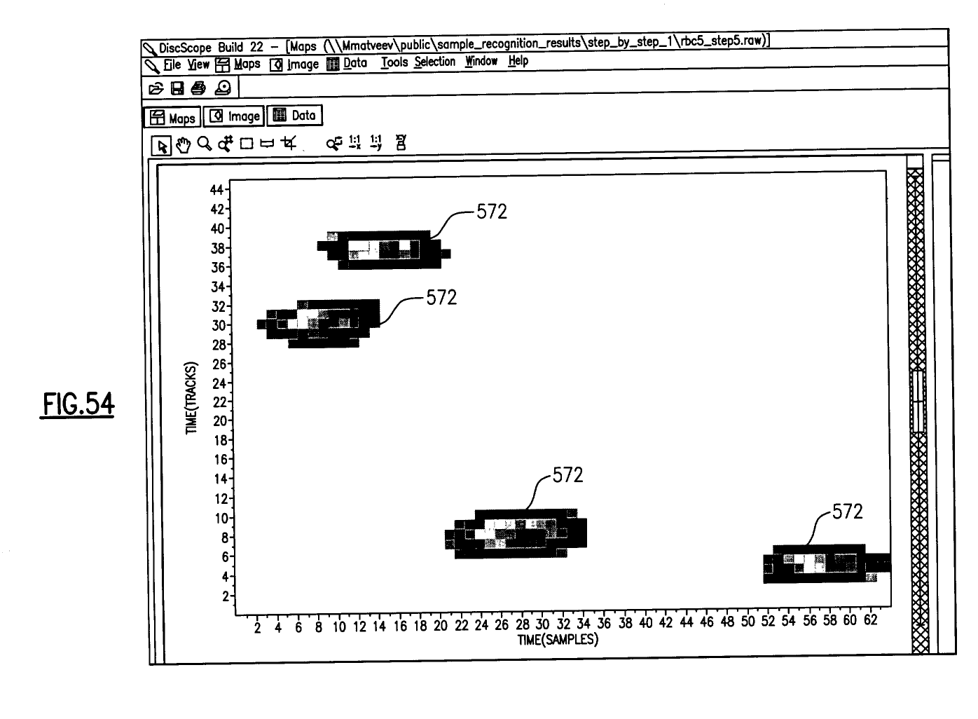

FIG. 54 shows the results of filling in the original data points. Imaged investigational data 572 mark captured cells. The advantage of this method lies in the ability to accurately extract cells. The extraction enables the user to measure the cell diameters and examine other features within cells such as the morphology of cell nuclei that have been stained. Further details relating to this type of application are discussed in commonly assigned U.S. patent application Ser. No. 10/xxx,xxx entitled “Nuclear Morphology Based Identification and Quantification of White Blood Cell Types Using Optical Bio-Disc Systems” filed Sep. 6, 2002 which is herein incorporated by reference.

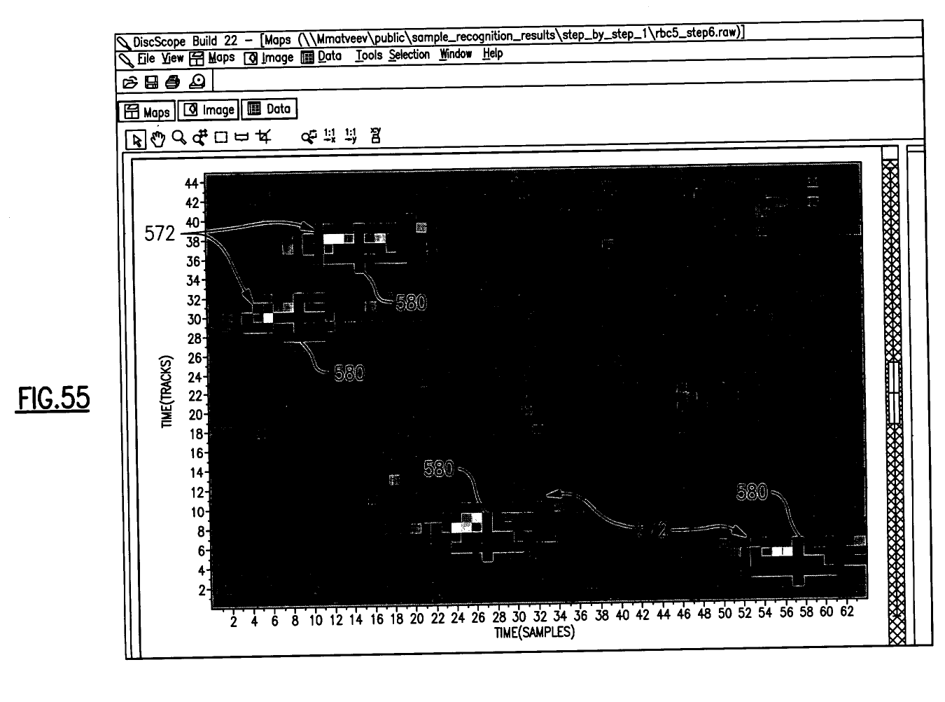

In addition to examining the imaged cells, the user can mark and count the cells by employing the user features of the present invention. FIG. 55 is a close-up view of an original pictorial representation of the investigational data from FIGS. 49-54 with the red blood cells marked by crosses, showing that they have been counted. Imaged investigational data 572 correspond to captured cells. Crosses 580 mark counted cells.

C. Alternative Algorithms

The present invention includes a number of alternative algorithms for handling special situations that may arise during the operations of cell counting.

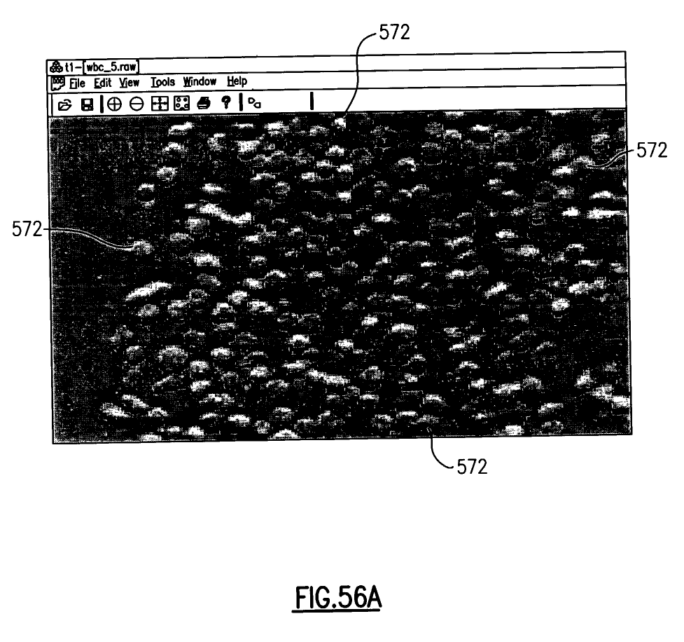

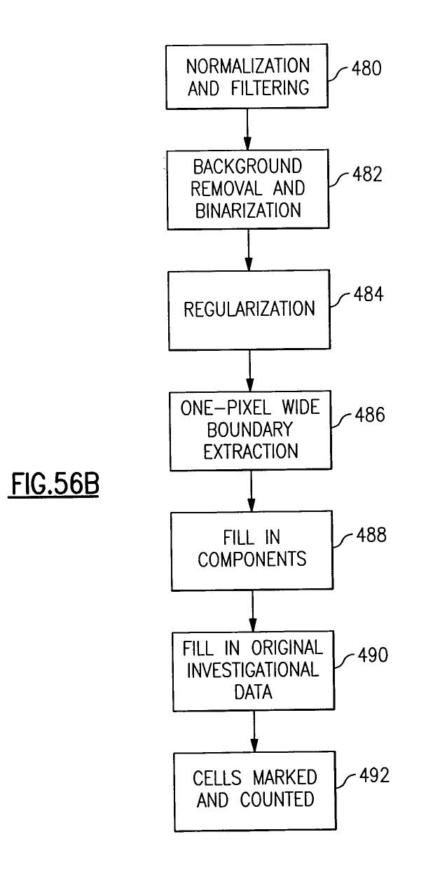

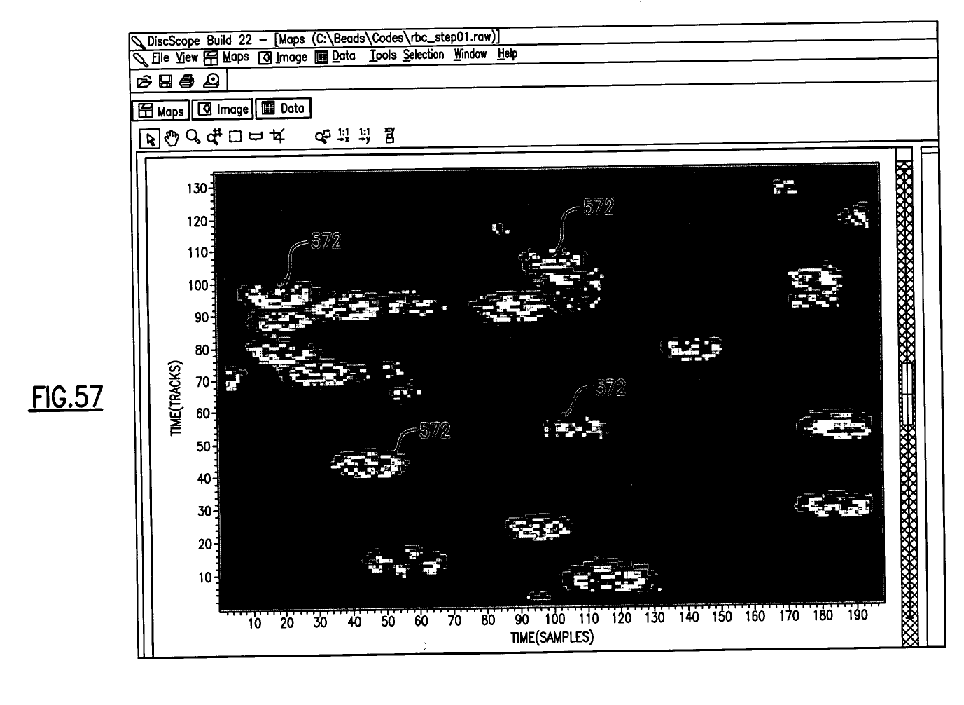

FIGS. 56A to 64 show an embodiment of the present invention that handles the case of counting cells without distinctive bright centers or dark rims. This method, termed “absolute value counting”, primarily deals with the case when cells appear to be without distinctive bright centers or dark rims. The bright center method relies on isolating high value areas (bright spots) in the investigational data to count cells. The dark rim method relies on isolating low value areas (dim spots) in the investigational data to count cells. This method of absolute value counting, in contrast, isolates areas that may not have distinctly high or low values detectable by either of the two prior methods, but nonetheless contain value patterns that can be distinguished from the background noise.

FIG. 56A is a pictorial screen shot of discrete cells before they are marked by crosses in accordance with the absolute value counting method. Imaged investigational data 572 represent captured cells. FIG. 56B is a flow chart depicting the steps involved in absolute value counting. In step 480, normalization and filtering are performed on the investigational data. FIG. 57 is a pictorial screen shot of discrete cells originally shown in FIG. 56A after the step of normalization and filtering (step 480, FIG. 56B). Imaged investigational data 572 corresponds to captured cells. The process of normalization and filtering is the same process as described herein above.

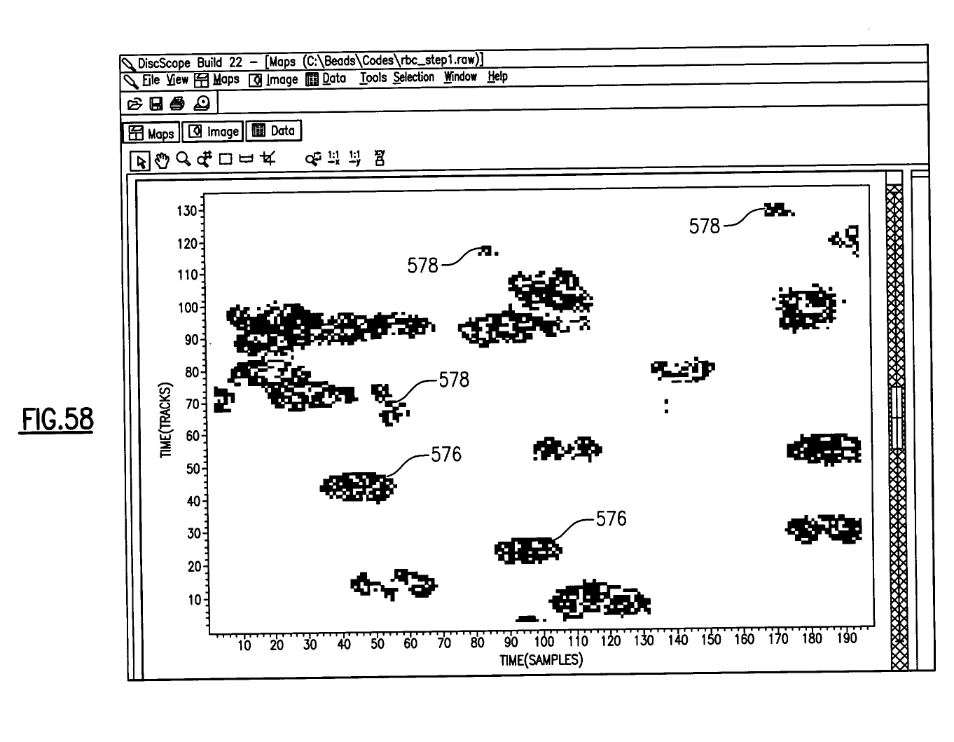

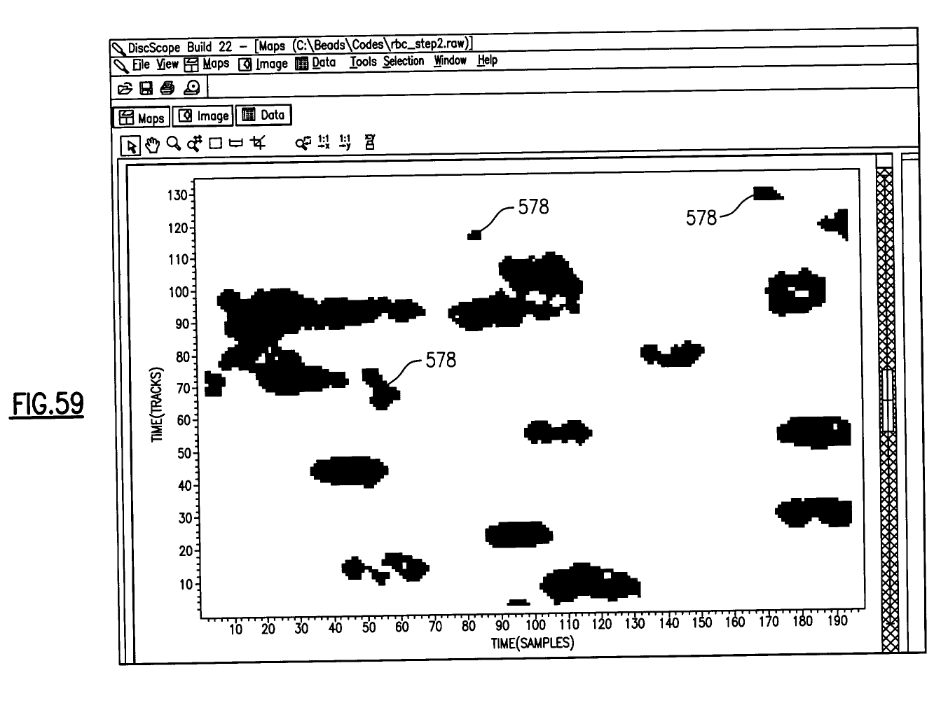

After normalization and filtering, the next step involves background removal and binarization (step 482, FIG. 56B). FIG. 58 is a pictorial screen shot of discrete cells originally shown in FIG. 56A after the step of background removal and binarization. Binary imaged data 576 mark data of investigational interest. In this case, the binary imaged data 576 indicates cells. Non-cell marking binary imaged data 578 point out data that do not represent cells. The background is removed to isolate where cells are located. Then binarization is performed on the investigational data to generate black and white points in the investigational data. The process of binarization proceeds as follows. The value of each point is first examined. When the difference between the value of the point and the background value is greater than a pre-determined threshold number, the point is declared black. Otherwise the point is declared white. In one embodiment, the threshold number is selected so that points that differ little from the background value (background or background noise) become white and points that differ much from the background value (dark or bright areas of cells) become black. In other embodiments, the threshold number is generated by a calibration mechanism that determines the background noise and background value based on varying conditions. Such condition may include the reflectivity of the bio-discs, the imbalance of the bio-discs within the bio-disc drives, the rattle, vibration or instability of the bio-discs, electrical noise, metalization of the bio-discs, the types of samples involved (white blood cells or others), and any other types of conditions requiring compensation or correction.

Regularization (step 484, FIG. 56B) is the next step in the process. FIG. 59 is a pictorial screen shot of discrete cells originally shown in FIG. 56A after the step of regularization. Binary imaged data 576 mark data of investigational interest. In this case, the binary imaged data 576 represents cells. Non-cell marking binary imaged data 578 point out data that do not represent cells. Regularization consists of erosion and expansion. Erosion is performed as follows. For an image P, a corresponding image P′ is constructed. A point X′ in P′ is declared white if (1) the corresponding point X in P is white, or (2) any neighbor of X is white. If neither condition is met, then X′ is declared black. P′ is the resulting image of erosion. Expansion operates in an opposite manner. For an image R, a corresponding image R′ is constructed. A point Y′ in R′ is declared black if (1) the corresponding point Y in R is black, or (2) any neighbor of Y is black. If neither condition is met, then Y′ is declared white. R′ is the resulting image of expansion. A composition of several erosions and expansions makes a binary image more regular (single black and single white points disappear).

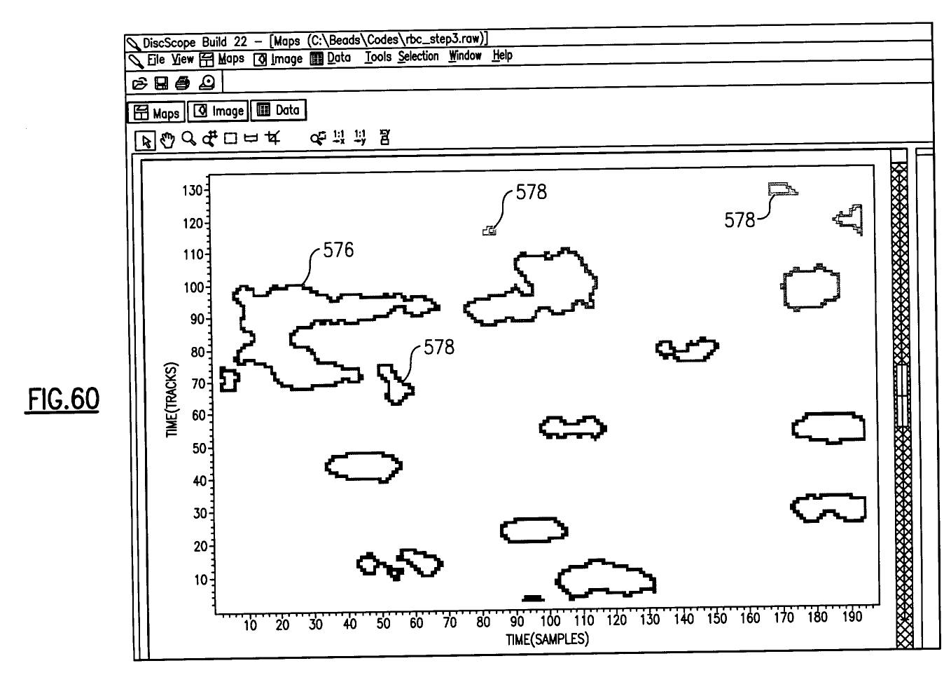

One-pixel wide boundary extraction is the next step. This extraction step is referenced step 486 in FIG. 56B. FIG. 60 is a pictorial screen shot of some of the discrete cells originally shown in FIG. 56A after applying the step 486 for one-pixel wide boundary extraction. Binary imaged data 576 mark data of investigational interest. In this case, the binary imaged data 576 correspond to cells. Non-cell marking binary imaged data 578 point out data that do not represent cells. In extracting the one-pixel wide boundary, all black points that have both black and white neighbors are selected to retain their black color. Black points without neighbor points of both colors are converted to white. This yields a rough boundary that is in places several pixels wide. Then the thinning process is applied to eliminate the extra points in the boundary until only one pixel is left in outlining the shapes in the investigational data. The thinning process starts by removing redundant black points from the boundary until only a one pixel wide line marks the boundary of each black area.

The thinning process starts with a rough boundary first. The rough boundary consists of all pixels which have neighbor pixels both inside and outside of the cell. After the rough boundary extraction, the data consists of three categories:

(1) pixels inside a cell,

(2) pixels marking the boundary of a cell, and

(3) pixels lying outside of a cell.

These three categories are related according to the following three conditions:

(A) all three are connected,

(B) (1) is disconnected from (3) by (2) and finally,

(C) each point in (2) has a neighbor in (1) or (2) but not both.

The thinning process then examines pixels in (2) one-by-one. If a pixel P in (2) has a neighbor in (1) or (3), then a check is performed to see if re-coloring P (e.g. black to white) from (2) to (1) or (3) still preserves conditions (A), (B), and (C). If so, then the re-coloring will be provided. This re-coloring is performed until a one-pixel wide boundary is obtained for each cell.

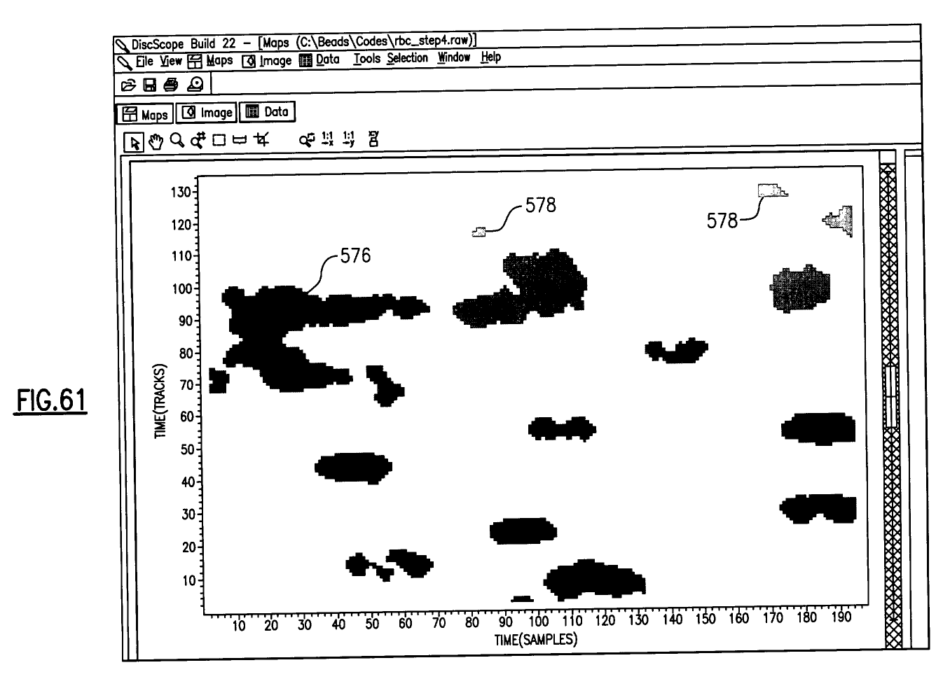

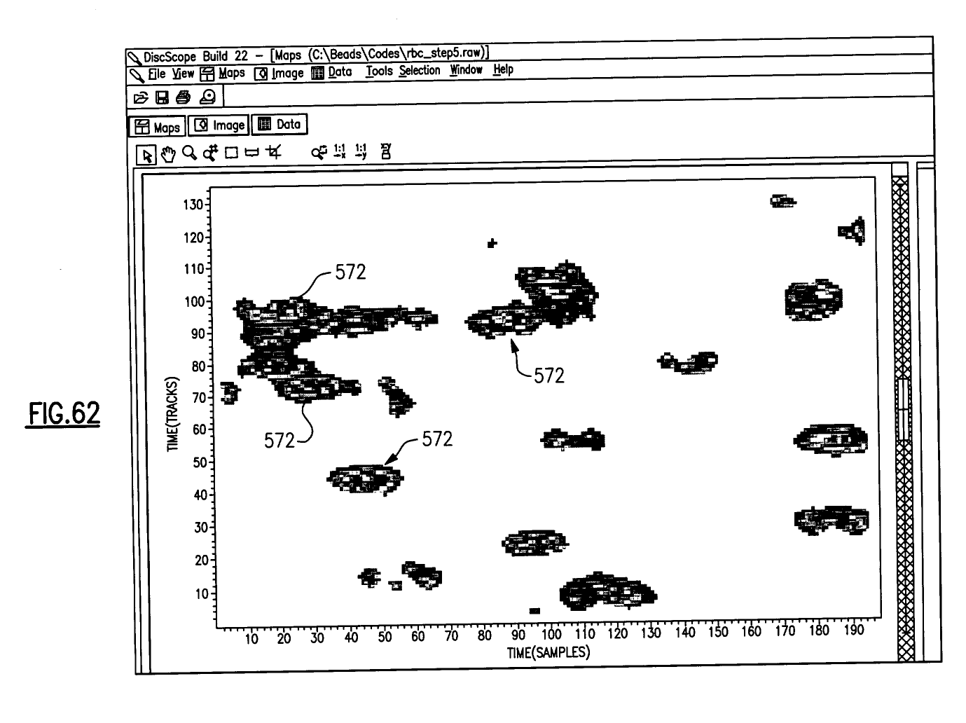

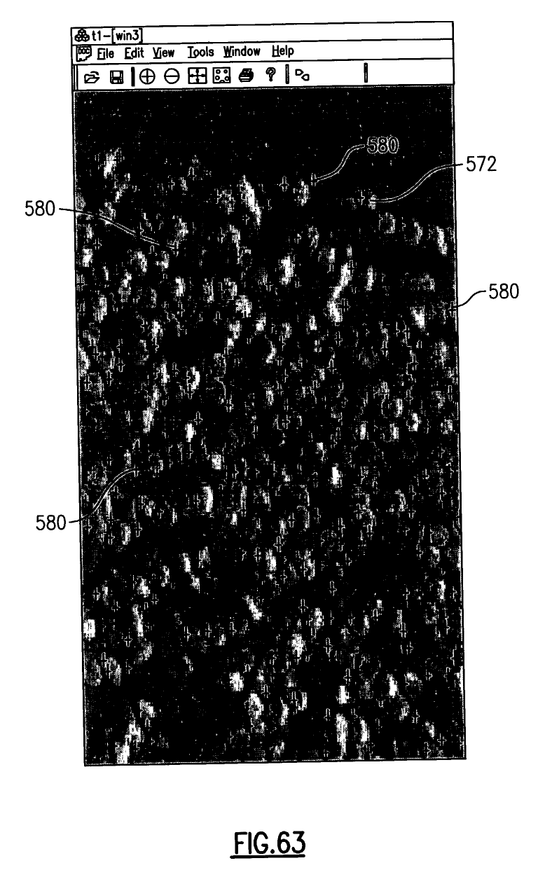

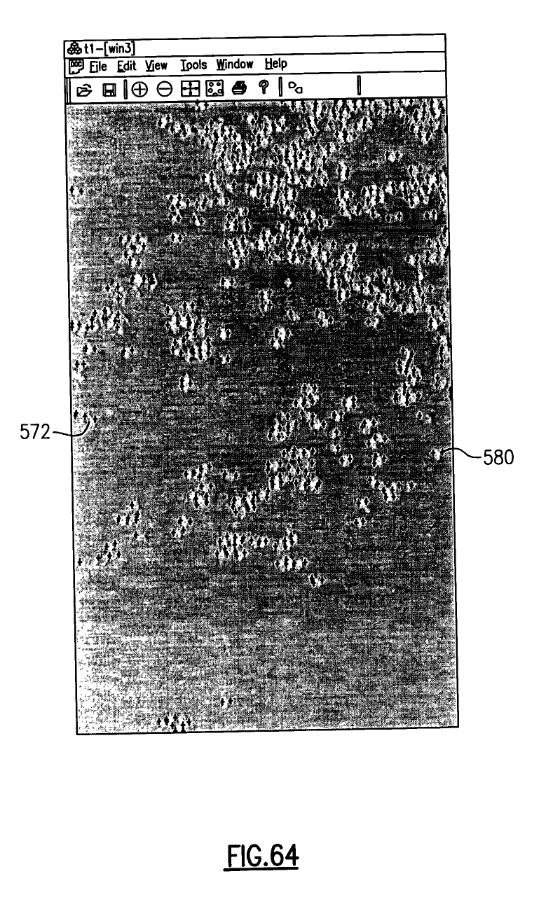

After the one-pixel wide boundary is extracted, the areas defined by the one-pixel wide boundaries are filled in with black points (step 488 of FIG. 56B). FIG. 61 is a pictorial screen shot of some of the discrete cells originally shown in FIG. 56A after performing step 488 to fill in components according to the present method. Binary imaged data 576 mark data of investigational interest. In this case, the binary imaged data 576 indicate cells. Non-cell marking binary imaged data 578 point out data that do not represent cells. Using this set of black and white points as a mask, the original data points are filled in to replace the black points (step 490, FIG. 56B). Thus the cell areas are isolated and can be counted. In one embodiment, convolution is applied to the isolated area in the investigational data and the local maxima are marked to identify cells. As with prior shown embodiments, convolution with circular neighborhood can be applied. Since the size of the circular neighborhood used in convolution is about the size of a cell, the local maxima can be determined to be center of cell. FIG. 62 is a pictorial screen shot of discrete cells originally shown in FIG. 56A after the step of filling in investigational data. Imaged investigational data 572 mark captured cells. Finally, FIG. 63 shows the cells as counted and marked by crosses according to step 492 of FIG. 56B. Imaged investigational data 572 correspond to captured cells. Crosses 580 mark counted cells. Whereas FIG. 63 shows only clumped cells, FIGS. 64 shows the method of absolute value counting applied to a sparsely packed sample with single cells and several clumped cell areas. Similarly, imaged investigational data 572 correspond to captured cells and crosses 580 mark counted cells.

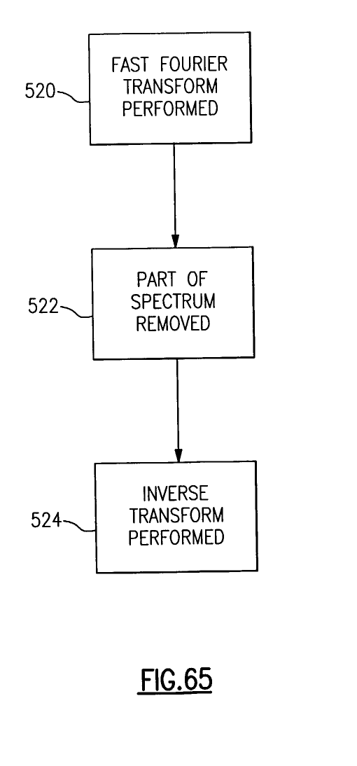

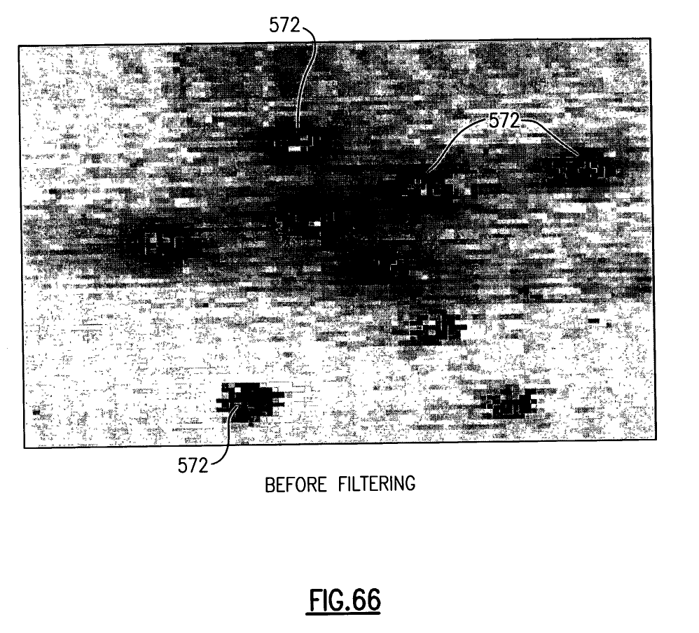

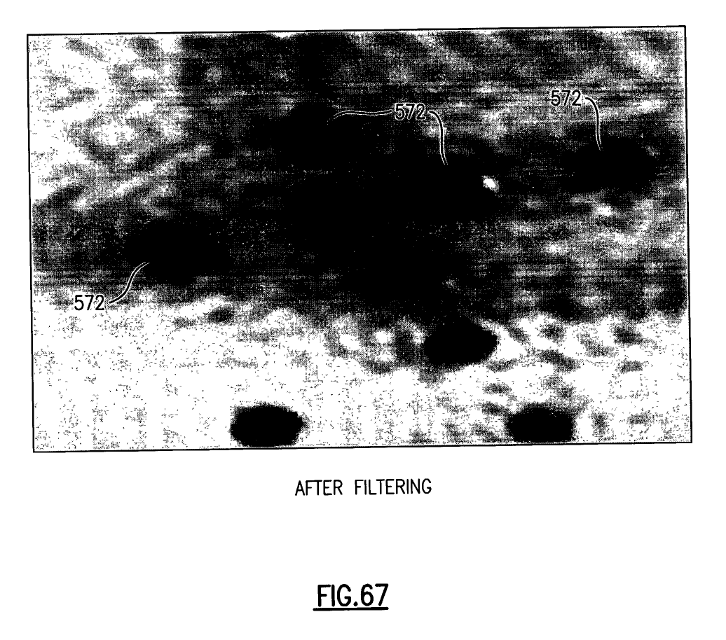

Another embodiment of the present invention improves the on-screen display of the evaluation rectangle that is marked off for cell counting. In the course of cell counting, it is sometimes desirable to display an image of the evaluation rectangle to the user. In addition to offering a visual presentation of the sample, a high quality image can aid the user in deciding what methods should be used to count and analyze the cells. For example, a clear image may alert of the user of the presence of many cells without bright centers. Thus, the user may choose to count cells also by the dark rim method. The present embodiment improves the quality of the image by way of a Fast Fourier Transform. As would be appreciated by those of skill in the art given the present disclosure, Fast Fourier Transform (FFT) is a variant of Fourier Transform (FT). Any variant of Fourier Transform can be applied here as well. Fourier Transform was discussed earlier in conjunction with background illumination uniformization. FIG. 65 offers a flow chart illustrating this embodiment of the present invention. In step 520, a Fast Fourier Transform is performed on the investigational data. The investigational data is converted to the frequency domain. Then in step 522, a part of the spectrum in the frequency domain is removed. Finally, in step 524, the inverse transform is performed. FIG. 66 shows an example investigational data before the Fast Fourier Transform. Imaged investigational data 572 represent captured cells. FIG. 67 shows that same investigational data after Fast Fourier Transform. Imaged investigational data 572 indicate captured cells and crosses 580 mark counted cells. The on-screen display is thus improved.

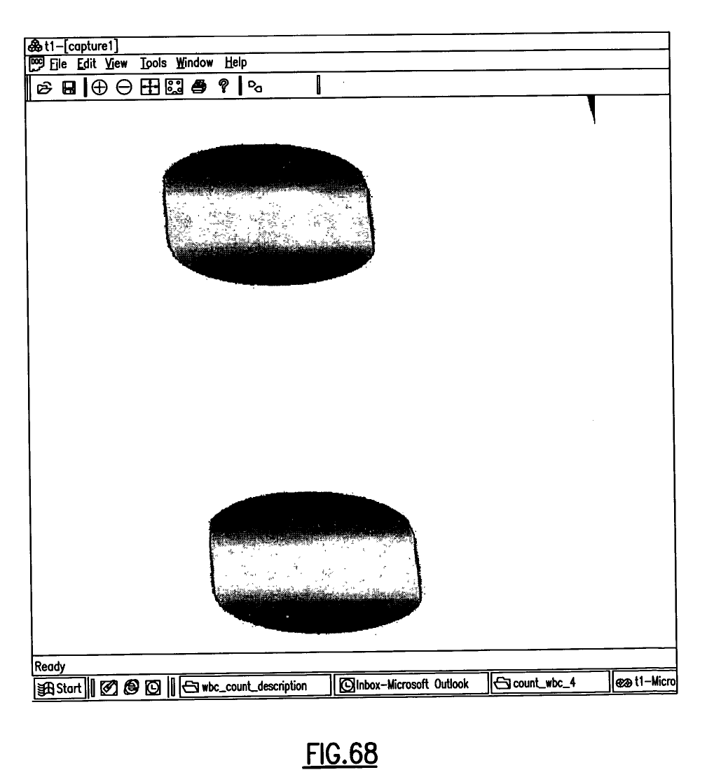

Another embodiment of the present invention handles the on-screen display of the window areas. In the course of cell counting on discs with windows, it is at times desirable to display the window areas to the user. Sometimes the images of the window areas are skewed, as shown in FIG. 68. To properly display the window areas, the skew needs to be corrected. The first step of the correction method finds the direction of the skew. This step of finding the skew takes advantage of the fact windows are bright areas in an otherwise dark background—shown here as white for convenience. To clarify the terms, a window is a rectangle with semicircles attached on its top and bottom, and the width of this rectangle is henceforward called the width of the window. The skew finding step proceeds as follows. First, the points from every line of the image are numerically differentiated. This means, for every point (x, y), the average value of all points in the interval from (x−dx, y) to (x, y) is subtracted from the average value in the interval from (x, y) till (x+dx, y). Here dx is a specific interval length chosen to eliminate noise.

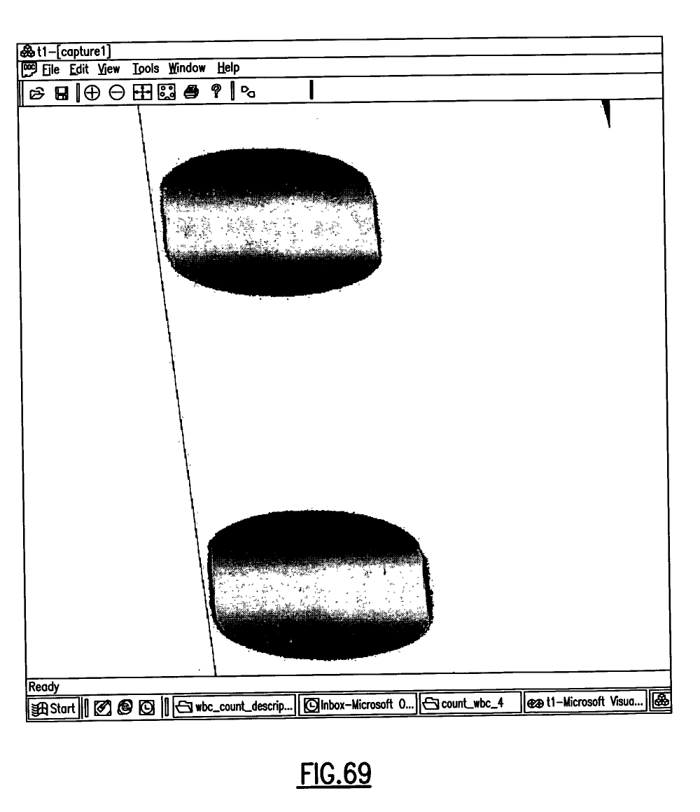

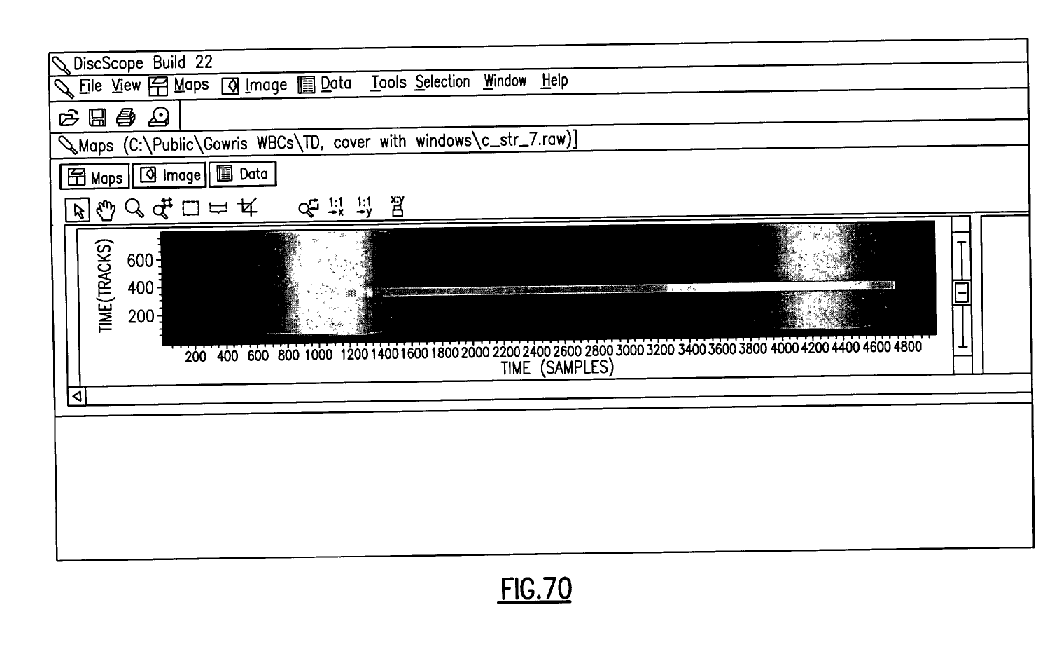

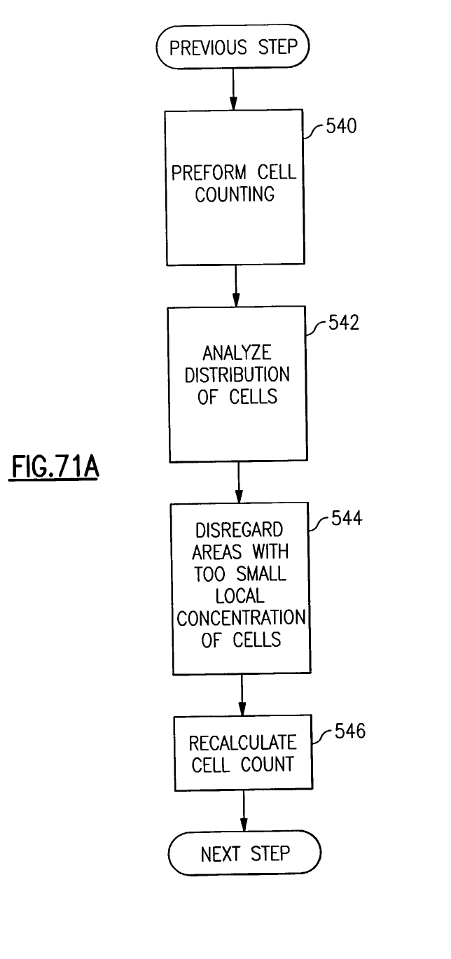

For lines of the image that coincide with windows, the result of this subtraction takes maximal value at the left border of the window and minimal value at the right border. This is because the average values in the bright windows are much higher than the average values in the dark background. In the lines of the image that lie outside of windows, these maximum and minimum values may happen elsewhere in arbitrary places, since the average values are from dark background with perhaps some noises. Taking advantage of this property, in the next step a distance D between the maximum and minimum values is calculated for every line. Next, the process chooses the lines with a D value that is close to the standard window width. After that, points of maxima are marked in these chosen lines. Finally a straight line is fitted across these points of maxima. The direction of this line is the direction of the skew. FIG. 69 shows the result with the direction line found. Finally the skew is corrected using this direction line as a guide and moving all the points accordingly. The image is properly aligned and displayed to the user as illustrated in FIG. 70.

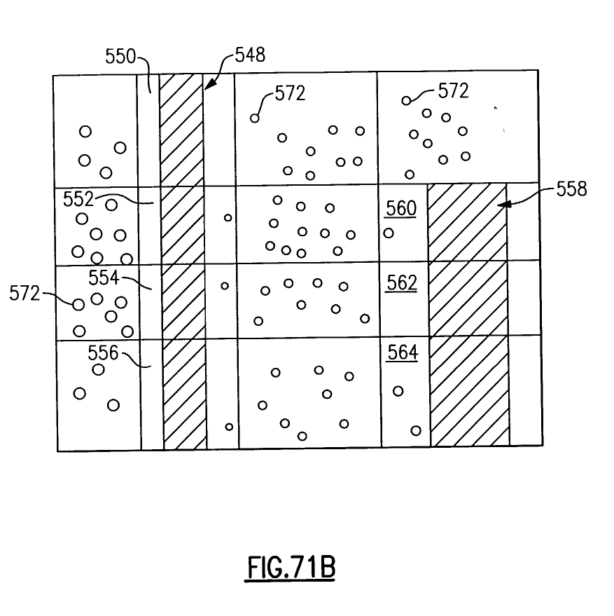

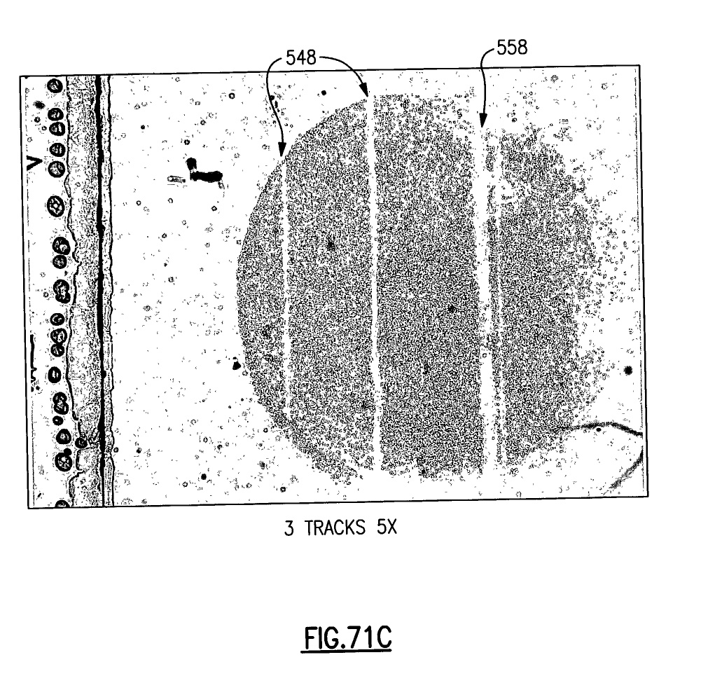

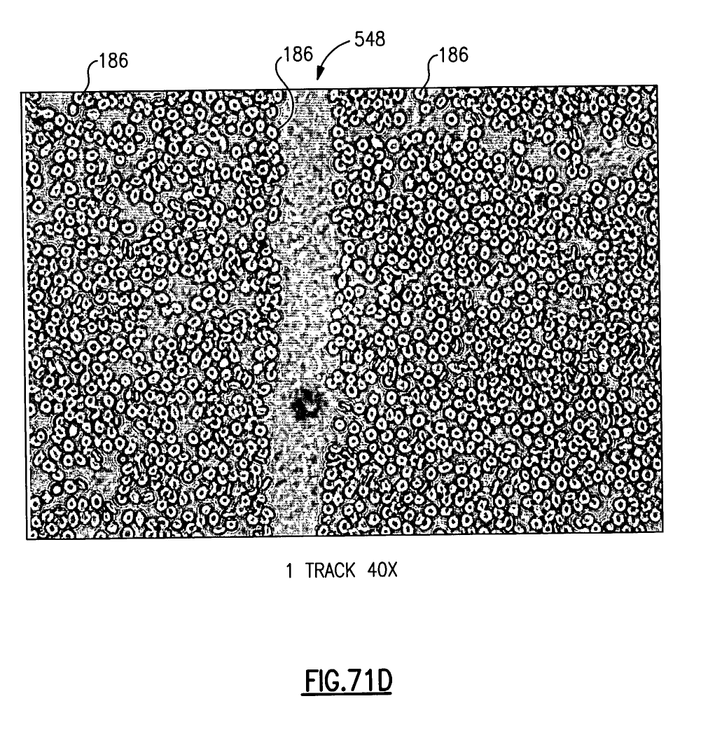

One embodiment of the present invention involves a method for removing bubble tracks from the investigational data. Sometimes an air bubble may become trapped in the channels on the disc. The air bubble may go through the sample and remove some of the cells along its path as it tracks across the capture zone. This removal of cells may cause an irregular cell distribution. Since the final result that is reported is in the form of number of cells per millimeter square (mm²) area, such irregular cell distribution must be corrected. The correction is performed as follows. FIG. 71A shows the flowchart of the process. In step 540, the cell counting is performed as before. Then the distribution of cells is analyzed in step 542. Areas with too small local concentration of cells are disregarded in step 544, since the cells in these areas are likely to have been wiped out by a bubble.

In one embodiment, the entire area that is being counted is divided into a grid of boxes. FIG. 71B shows such as example. Imaged investigational data 572 mark captured cells (represented by circles). Bubble track 548 runs through the area bounded by boxes 550, 552, 554, and 556. Another bubble track 558, being wider than track 548, runs through the area bounded by boxes 580, 582, and 564. The areas bounded by these boxes are disregarded. Once such areas are disregarded, the cell count is recalculated in step 546 of FIG. 71A. FIGS. 71C and 71D show examples of bubble tracks. FIG. 71C shows bubble tracks (including tracks 548 and 558) through a sample as seen under microscope power 5×. FIG. 71D shows another example where a bubble track 548 through a sample is illustrated under microscope power 40×.

III. White Blood Cell Count Method

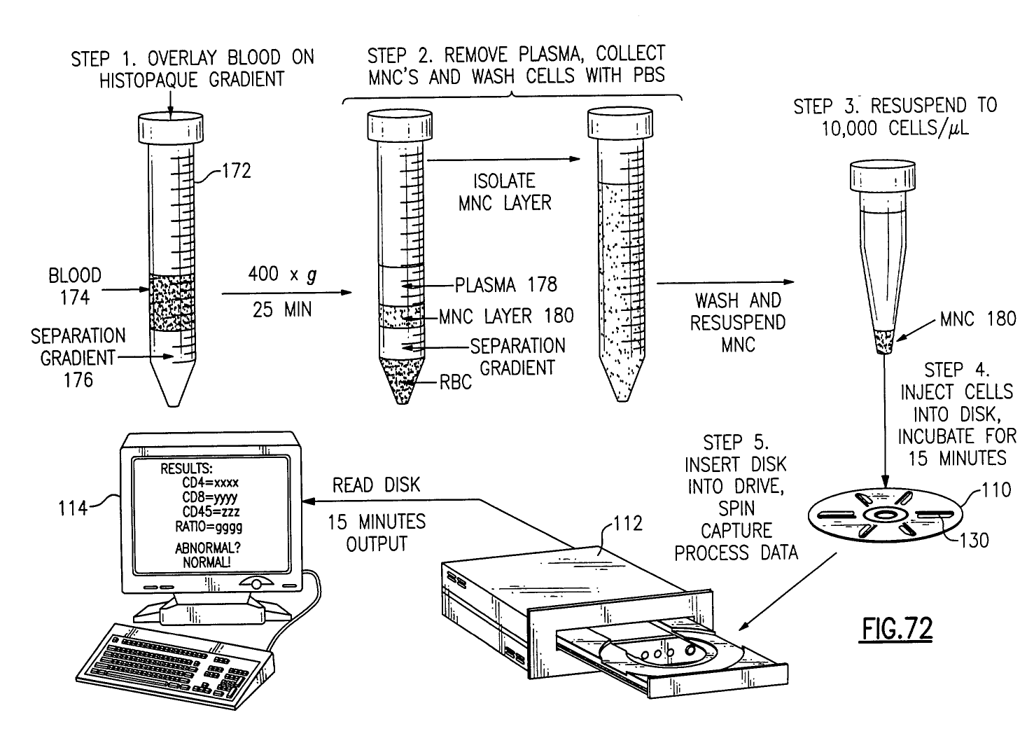

FIG. 72 offers an example of how a generic homogeneous solid phase cell capture assay for the rapid determination of absolute number of CD4+ and CD8+ T-lymphocyte populations and ratio of CD4+/CD8+ lymphocytes in blood samples may be performed utilizing the methods of the invention. The test, which is run within small flow channels incorporated into a bio-disc, determines the number of CD4+, CD8+, CD2+, CD3+, CD19+, and CD45+ cells captured by the specific antibodies on the capture zones in 7 to 15 μl of mononuclear cells (MNC) isolated from whole blood. The test is based upon the principle of specific cell capture on localized locations on the disc. Several specific cell capture zones are created on the disc by localized application of capture chemistries based upon monoclonal or polyclonal antibodies to particular blood cell surface antigens. Upon flooding the 25 to 100 μl chambers with the MNC blood (10,000 to 30,000 cells/μl), cells expressing CD4, CD8, CD2, CD3, CD19, and CD45 antigens are captured in the capture zones within the disc. Also incorporated within the capture zones are defined negative and positive control areas.

In step 1 of FIG. 72, blood (4 to 8 ml) is collected directly into a 4 or 8 ml Becton Dickinson CPT Vacutainer™ and an anticoagulant such as EDTA, ACD, or heparin. In an equivalent step of another embodiment of the invention, 3 ml of blood in anticoagulant is overlaid into a tube 172 containing a separation gradient 176 such as Histopaque 1077. In any case, the blood sample 174 is preferably used within two hours of collection. The tube 172 containing the separation gradient 176 with blood sample 174 overlay is centrifuged at 1,500 to 1,800 RCFs (2,800 rpm) in a biohazard centrifuge with horizontal rotor and swing out buckets for 25 minutes at room temperature. After centrifugation, the plasma layer 178 is removed (step 2), leaving about 2 mm of plasma above the mononuclear cell (MNC) fraction 180. The MNC layer 180 is collected and washed with phosphate buffer saline (PBS). Cells are pelleted by centrifugation at 300 RCFs (1200 rpm) for 10 minutes at room temperature to remove any remaining platelets. The supernatant is removed and the MNC pellet 180 is re-suspended in PBS by tapping the tube gently. The final pellet 180 is re-suspended (step 3) to a cell count of 10,000 to 30,000 cells/μl depending upon the height of the flow channel 130 of the bio-disc 110.

The flow channel 130 of a bio-disc 110 is flooded with 7 μl of the MNC suspension, and the inlet ports 122 and vent ports 124 (FIGS. 3 and 5) of the chamber are sealed with sealing tabs (step 4). The bio-disc 110 is incubated for 15 minutes at room temperature, and then scanned using a 780 nm laser in an optical drive 112 to image the capture field (step 5). It should be understood that if a transmissive bio-disc 110 is used, optical drive 112 optionally includes a top detector 158 (FIG. 10A) to image the capture field. Software is preferably encoded on the disc to instruct the drive to automatically perform the following acts: (a) centrifuge the disc to spin off excess unbound cells in one or more stages, (b) image specific capture windows on a display monitor 114, and (c) process data. Data processing includes, but is not limited to counting the specifically captured cells in each capture zone and deriving the ratio of CD4+/CD8+ or any other desired count or ration that may be programmed accordingly.

As is further illustrated in FIG. 72, the present invention is directed to a method of performing a cluster designation count with an optical disc and disc drive. The method includes the steps of providing a blood sample in a first tube containing a separation gradient, rotating the first tube at a time and speed sufficient to separate the blood sample into layers, resuspending a MNC layer that contains T-cells to form a MNC suspension, providing a sample of the MNC suspension on a disc surface that includes at least one capture zone containing at least one capture agent, loading the disc into an optical reader, rotating the disc, directing an incident beam of electromagnetic radiation to the capture zone, detecting a beam of electromagnetic radiation formed after interacting with the disc at the capture zone, converting the detected beam into an output signal, and analyzing the output signal to extract information relating to the number of cells captured at the capture zone. In one embodiment of this method, the optical disc is constructed with a reflective layer such that light directed to the capture zone and interacting with a cell is reflected. In another embodiment of this method, the optical disc is constructed such that light directed to the capture zone and interacting with a cell is transmitted through the optical disc.

During the analyzing/processing step, the software reads across each capture zone image and marks cell images as it encounters them. For example, following an estimation of the number of CD4+ and CD8+ cells, the software calculates the ratio of CD4+/CD8+ cells and displays both the absolute numbers of cells in CD4+, CD8+, CD3+, and CD45+ capture zones per microliter of whole blood and also the CD4+/CD8+ ratio. The entire process takes about 12 minutes from inserting the disc into the optical drive to obtaining the numbers and ratios.

In one embodiment, the disc is a forward Wobble Set FDL21:13707 or FDL21:1270 CD-R disc coated with 300 nm of gold as the encoded information layer. On a reflective disc, viewing windows of size 2×1 mm oval are etched out of the reflective layer by known lithography techniques. In some designs of transmissive disc, no separate viewing windows are etched, and the entire disc is available for use. The adhesive layer is Fraylock adhesive DBL 201 Rev C 3M94661. The cover is a clear disc with 48 sample inlets with a diameter of 0.040 inches located equidistantly at radius 26 mm. The data disc is scanned and read with the software at speed 4× and sample rate 2.67 MHz using CD4+/CD8+ counting software.

IV. Conclusion

Thus methods and apparatus for imaging cells in laboratory samples and analyzing such images are described in conjunction with one or more specific embodiments. While this invention has been described in detail with reference to certain preferred embodiments, it should be appreciated that the present invention is not limited to those precise embodiments. Rather, in view of the present disclosure which describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

We claim:

1. A method of counting cells, said method comprising the steps of:
obtaining investigational data of a sample with cells;
selecting an evaluation rectangle in said investigational data;
enhancing said investigational data within said evaluation rectangle; and
counting cells within said evaluation rectangle.

2. The method of claim 1 wherein said step of selecting further comprises the step of selecting a custom size for said evaluation rectangle.

3. The method of claim 1 wherein said step of selecting selects a plurality of evaluation rectangles.

4. The method of claim 1 wherein said step of enhancing said investigational data area further comprises the steps of:
performing background illumination uniformization on said investigational data;
performing normalization on said investigational data; and
filtering said investigational data.

5. The method of claim 4 wherein said step of performing background illumination uniformization further comprises the steps of:
choosing a size for a neighborhood rectangle;
picking a point in said investigational data;
performing horizontal scanning to calculate a first sliding average for all neighbor points located within said neighborhood rectangle centered at said point;
performing vertical scanning to calculate a second sliding average for all neighbor points located within said neighborhood rectangle centered at said point;
combining said first sliding average and second sliding average to create an overall average;
reassigning the original value of said point to a resultant value calculated by obtaining the difference between said overall average and said original value and adding said difference to a background value; and
repeating said steps of performing horizontal scanning, performing vertical scanning, combining two said averages and reassigning the original value for all points in said investigational data.

6. The method of claim 4 wherein said step of performing background illumination uniformization further comprises the steps of:
performing Fourier Transform on said investigational data to produce frequency domain functions;
removing low wavelength functions from said frequency domain functions;
removing high wavelength functions from said frequency domain functions; and
performing inverse transform on said frequency domain functions to obtain a modified version of said investigational data.

7. The method of claim 4 wherein said step of performing normalization further comprises the steps of:
calculating an average and a standard deviation of the value of all points in said investigational data;
normalizing said value of all points in said investigational data using said average and said standard deviation; and
truncating said value of some points if necessary.

8. The method of claim 4 wherein said step of filtering further comprises the steps of:
choosing a size for a neighborhood rectangle;
picking a point in said investigational data;
finding all sufficiently distinct points located in said neighborhood rectangle centered at said point;
reassigning the value of said point if the number of said sufficiently distinct points is greater than a pre-determined filtering criteria; and
repeating said steps of finding all sufficiently distinct points and reassigning the value for all points in said investigational data.

9. The method of claim 4 further comprising the steps of:
removing undesirable components from said investigational data after said filtering step; and
repeating said step of performing background illumination, said step of performing normalization and said step of filtering.

10. The method of 9 wherein said step of removing undesirable components further comprises the steps of:
selecting a threshold value;
performing binarization on said investigational data using said threshold value;
performing regularization on said investigational data;
extracting connected components;
selecting a size threshold; and
removing components that fail to meet said size threshold.

11. The method of 10 wherein said step of performing regularization further comprises the step of performing a plurality of erosion and expansion.

12. The method of 10 wherein said step of extracting connected components further comprises the steps of:
assigning initial component numbers to all black points on said investigational data;
picking a starting point;
setting an scan direction;
scanning all points of said investigational data to reassign the component number of each of said black points to match the component number of adjacent black points;
altering said scan direction according to a set of pre-determined rules; and
repeating said steps of scanning and altering so that said component numbers of connected black points become the same.

13. The method of claim 1 wherein said step of counting cells shown in said evaluation rectangle further comprises the steps of:
performing convolution on said investigational data;
searching for a plurality of local maxima of said investigational data;
removing redundant local maxima from said plurality of local maxima;
declaring remaining maxima to be bright centers of cells; and
counting cells by recognizing said bright centers of cells.

14. The method of claim 13 wherein said step of performing convolution uses an indicator function that defines a circular neighborhood wherein said circular neighborhood bounds the expected size of a cell.

15. The method of claim 13 wherein said step of performing convolution uses a Gaussian indicator function.

16. The method of claim 13 wherein said step of removing redundant local maxima further comprises the steps of:
selecting a distance threshold; and
using said distance threshold to determine whether a local maxima is redundant.

17. The method of claim 13 further including a step of performing a statistical analysis comprising the steps of:
obtaining distribution of cells based of counted cells; and
estimating cell counts in areas where cells are clumped or visibility is low.

18. The method of claim 13 further comprising the steps of:
re-sampling said investigational data at a higher resolution; and
repeating said steps of performing convolution, searching for a plurality of local maxima, removing redundant local maxima, declaring remaining maxima to be bright centers of cells and counting cells by recognizing said bright centers of cells.

19. The method of claim 13 further comprising the steps of:
removing said cells counted by bright centers from said investigational data;
counting cells by recognizing dark dims; and
adding total from said step of counting cells by recognizing bright centers to total from said step of counting by recognizing dark rims.

20. The method of claim 19 wherein said step of counting cells by recognizing dark rims further comprises the steps of:
performing inversion on said investigational data;
performing a plurality of convolutions with shifted rings;
summing results from said plurality of convolutions;
finding local maxima;
declaring maxima to be centers of cells; and
counting said centers of cells.

21. The method of claim 20 wherein said step of performing a plurality of convolutions performs convolutions without shifted rings.

22. The method of claim 20 wherein said step of performing convolution uses a Gaussian indicator function.

23. The method of claim 20 wherein said step of performing convolution uses a smoothing function.

24. The method of claim 1 wherein said step of counting cells shown in said evaluation rectangle further comprises the steps of:
performing inversion on said investigational data;
performing a plurality of convolutions with shifted rings;
summing results from said plurality of convolutions;
finding local maxima;
declaring maxima to be centers of cells; and
counting said centers of cells.

25. The method of claim 24 wherein said step of performing a plurality of convolutions performs convolutions without shifted rings.

26. The method of claim 1 wherein said step of enhancing further comprises the steps of:
performing normalizing on said investigational data;
performing filtering on said investigational data;
selecting a threshold number;
performing binarization on said investigational data by determining if said investigational data differs from a set background value by a value greater than said threshold number;
performing regularization on said investigational data;
extracting one-pixel wide boundaries in said investigational data;
filling in areas defined by said one-pixel boundaries with investigational data; and
applying convolution in said filled in areas.

27. The method of claim 1 further comprising the step of displaying on a computer monitor image representation of said investigational data.

28. The method of claim 27 wherein said step of displaying further comprises the steps of:
performing fast Fourier Transform on said investigational data to generate investigational data in the frequency domain;
removing part of the spectrum in the frequency domain; and
performing inverse transform on said investigational data in the frequency domain to enhance said investigational data for display.

29. The method of claim 1 wherein said step of obtaining investigational data of a sample with cells comprises the steps of:
providing a blood sample on an optical disc surface, said surface including one or more capture zones with one or more capture agents;
loading said optical disc into an optical reader;
rotating said optical disc;
directing, from a light source, an incident beam of electromagnetic radiation to one of said capture zones;
detecting, with a detector, a resultant beam of electromagnetic radiation formed after said incident beam interact with the disc at said capture zone;
converting the detected beam into an analog output signal; and
converting said analog output signals into digital data containing cells captured at said capture zone.

30. The method of claim 29 wherein said step of converting said analog output to said digital data further comprises the steps of:
sampling amplitudes of said analog signals at fixed intervals;
recording said sampling amplitudes in an one-dimensional array;
creating a plurality of one-dimensional arrays using said steps of sampling and recording; and
combining said plurality of one-dimensional arrays to create a two-dimensional array containing digital data of said sample.

31. The method according to claim 29 wherein said optical disc is constructed with a reflective layer such that light directed to said capture is reflected to said detector.

32. The method of claim 31 where said detector is a bottom detector.

33. The method according to claim 29 wherein the optical disc is constructed such that light directed to said capture zone is transmitted through said optical disc, said disc being between said light source and said detector.

34. The method of claim 33 wherein said detector is a top detector.

35. The method of claim 33 wherein said detector is a split detector.

36. The method of claim 29 wherein said one or more capture zones are located within one or more chambers within said optical disc.

37. The method of claim 29 wherein said optical disc comprises a plurality of windows that correspond to said capture zones.

38. The method of claim 37 wherein said step of selecting evaluation rectangles step further comprises the steps of:
finding one of said plurality of windows in said investigational data; and
cropping an evaluation rectangle of standard size inside said window.

39. The method of 37 wherein said step of finding one of said plurality of windows further comprises the steps of:
performing compression on said investigational data;
performing threshold evaluation on said investigational data;
performing binarization on said investigational data;
performing regularization on said investigational data;
extracting connected components from said investigational data; and
finding a component from said connected components that corresponds to a window.

40. The method of 39 wherein said step of extracting connected components further comprises the steps of:
assigning initial component numbers to all black points on said investigational data;
picking a starting point;
setting an scan direction;
scanning all points of said investigational data to reassign the component number of each of said black points to match the component number of adjacent black points;
altering said scan direction according to a set of pre-determined rules; and
repeating said steps of scanning and altering so that said component numbers of connected black points become the same.

41. The method of claim 29 wherein the surface of said optical disc contains dark spots that mark the location of said captured zones.

42. The method of claim 41 wherein said step of selecting evaluation rectangles further comprises:
finding one of said dark spots in said investigational data; and
creating an evaluation rectangle of standard size with a center located at a point found by shifting a pre-determined distance from said dark spot.

43. The method of claim 42 wherein said step of finding one of said dark spots further comprises the steps of:
performing compression on said investigational data;
performing threshold evaluation on said investigational data;
performing binarization on said investigational data;
performing regularization on said investigational data;
extracting connected components from said investigational data; and
finding a component from said connected components that corresponds to a dark spot.

44. The method of 43 wherein said step of extracting connected components further comprises the steps of:
assigning initial component numbers to all black points on said investigational data;
picking a starting point;
setting an scan direction;
scanning all points of said investigational data to reassign the component number of each of said black points to match the component number of adjacent black points;
altering said scan direction according to a set of pre-determined rules; and
repeating said steps of scanning and altering so that said component numbers of connected black points become the same.

45. The method of claim 42 wherein said step of finding one of said dark spots further comprises the step of reading location information

46. The method of 29 wherein said optical disc contains computer readable location information for locating said capture zone.

47. The method of 37 further comprising the step of displaying image of said window on a computer monitor.

48. The method of 47 where said step of displaying said image of said window further comprises:
determining if said image is skewed;
finding the direction of the skew; and
correcting the skew of said image.

49. The method of claim 1 wherein said step of obtaining investigational data of said sample with cells comprises retrieving previously stored investigational data of samples from an archive.

50. The method of claim 49 wherein said archive catalogs said stored investigational data according to characteristics of patients.

51. The method of claim 50 wherein said step of retrieving previously stored investigational data of samples further comprises the step of selecting samples matching a plurality of criteria chosen from said characteristics of patients so that a population health trends study is conducted.

52. The method of claim 1 further comprising the step of outputting results from said step of counting cells.

53. The method of claim 52 wherein said cells are white blood cells.

54. The method of claim 53 wherein said results include counts for CD4+ cells and CD8+ cells, and a ratio of CD4+ to CD8+ cells.

55. The method of claim 54 wherein said results further include counts for CD3+ cells and CD45+ cells.

56. The method of claim 1 wherein said step of counting cells further comprises the steps of:
analyzing the distribution of cells for bubble tracks;
disregarding areas with too small local cell concentration; and
recalculating cell counts.
